(19) United States
(12) Patent Application Publication
(10) Pub. No.: US 2005/0227252 A1
(43) Pub. Date: Oct. 13, 2005

(54) Diffraction grating-based encoded articles for multiplexed experiments

(76) Inventors: John A. Moon, Wallingford, CT (US); Martin A. Putnam, Cheshire, CT (US); Michel Perbost, Bethany, CT (US); John Joseph Quinn, Madison, CT (US); Mary Trounstine, New Haven, CT (US)

Correspondence Address: Illumina, Inc.;CyVera Corporation Subsidiary, C/O Portfolio IP, P. O. Box 52050, Minneapolis, MN, 55402, US

(21) Appl. No.: 10/990,057

(22) Filed: Nov. 15, 2004

Related U.S. Application Data
(63) Continuation-in-part of application No. 10/661,234, filed on Sep. 12, 2003.
Application No. 10/661,234 is a continuation-in-part of application No. 10/645,689, filed on Aug. 20, 2003.
(60) Provisional application No. 60/519,932, filed on Nov. 14, 2003.
(60) Provisional application No. 60/555,449, filed on Mar. 22, 2004.
(60) Provisional application No. 60/602,427, filed on Aug. 18, 2004.
(60) Provisional application No. 60/611,676, filed on Sep. 20, 2004.
(60) Provisional application No. 60/546,435, filed on Feb. 19, 2004.
(60) Provisional application No. 60/610,059, filed on Sep. 13, 2004.
(60) Provisional application No. 60/405,087, filed on Aug. 20, 2002.
(60) Provisional application No. 60/410,541, filed on Sep. 12, 2002.

(51) Int. Cl. C12Q 1/68; G01N 33/53; C12M 1/34

(52) U.S. Cl. 435/6; 435/7.1; 435/287.2

(57) ABSTRACT

The present invention provides methods and compositions directed toward assays of a broad range of analytes using specific targeting chemicals that bind to the analytes. The assays are founded on the use of coded assay articles to which the targeting chemicals are attached. Additionally the codes are such that they are interrogated and determined in real time. The target is analyzed as to identity, presence and quantity in real time. The methods and compositions of the invention are highly suitable for use in high-complexity multiplexed assay systems. All the methods and compositions are based on assay article that includes an optical substrate to which the chemical is bound, and in which is disposed at least one diffraction grating. The grating provides an output optical signal when illuminated by an incident light signal which is indicative of the code in the substrate. In general, coded assay article or sets thereof are employed in assay methods, including multiplexed assay methods, according to which a sample is contacted with an article or a set, and any analytes that bind to the attached chemical are identified according to the code, detected and/or quantitated.

### CROSS REFERENCES TO RELATED APPLICATIONS

This application claims the benefit of US Provisional Patent Applications, Ser. No. 60/519,932 (CyVera Docket No. CV-0052 PR), filed Nov. 14, 2003; Ser. No. 60/555,449 (CyVera Docket No. CV-0072 PR), filed Mar. 22, 2004; Ser. No. 60/602,427 (CyVera Docket No. CV-0076 PR), filed Aug. 18, 2004; Ser. No. 60/661,205 (CyVera Docket No. CV-0085 PR), filed Sep. 17, 2004; Ser. No. 60/611,676 (CyVera Docket No. CV-0091 PR), filed Sep. 20, 2004; Ser. No. 60/546,435 (CyVera Docket No. CV-0053 PR), filed Feb. 19, 2004; Ser. No. 60/610,059 (CyVera Docket No. CV-0083 PR), filed Sep. 13, 2004; and is a continuation-in-part of U.S. patent application Ser. No. 10/661,234 (CiDRA Docket No. CV-0038A), filed Sep. 12, 2003, which is a continuation-in-part of U.S. patent application Ser. No. 10/645,689 (CyVera Docket No. CC-0638), filed Aug. 20, 2003, which claimed the benefit of US provisional applications, Ser. No. 60/405,087 (CyVera Docket No. CV-0005PR/Prior CC-0429PR) filed Aug. 20, 2002 and Ser. No. 60/410,541 (CyVera Docket No. CV-0012PR/Prior CC-0543 PR), filed Sep. 12, 2002; and is a continuation-in-part of U.S. patent application Ser. No. 10/661,031 (CyVera Docket No. CV-0039A), which is a continuation-in-part of U.S. patent application Ser. No. 10/645,686 (CyVera Docket No. CC-0639), filed Aug. 20, 2003, which claimed the benefit of U.S. provisional application Ser. No. 60/405,087 (CyVera Docket No. CV-0005PR/Prior CC-0429PR) filed Aug. 20, 2002 and Ser. No. 60/410,541 (CyVera Docket No. CV-0012PR/Prior CC-0543 PR), filed Sep. 12, 2002; and is a continuation-in-part of U.S. patent application Ser. No. 10/661,082 (CyVera Docket No. CV-0040), filed Sep. 12, 2003; and is a continuation-in-part of U.S. patent application Ser. No. 10/661,115 (CyVera Docket No. CV-0041), filed Sep. 12, 2003; and a continuation-in-part of U.S. patent application Ser. No. 10/661,836 (CyVera Docket No. CV-0042), and a continuation-in-part of U.S. patent application Ser. No. 10/763,995 (CyVera Docket No. CV-0054) filed Jan. 22, 2004, all of which are incorporated herein by reference in their entirety.

The following cases contain subject matter related to that disclosed herein and are incorporated herein by reference in their entirety: U.S. patent application Ser. No. 10/661,254 (Docket No. CV-0043), filed Sep. 12, 2003, entitled “Chemical Synthesis Using Diffraction Grating-based Encoded Optical Elements”; U.S. patent application Ser. No. 10/661,116 (Docket No. CV-0044), filed Sep. 12, 2003, entitled “Method of Manufacturing of a Diffraction grating-based identification Element”; and U.S. Provisional Patent Application Ser. No. 60/609,712 (Docket No. CV-0084 PR), filed Sep. 13, 2004, entitled “Method and Apparatus for labeling using Diffraction Grating based Encoded Optical Elements”.

### TECHNICAL FIELD

This invention relates generally to assay methods for detecting analytes of interest in biotechnology, clinical medicine and related areas, and to the compositions employed in carrying out the assays. Somewhat more specifically, the invention relates to coded solid state-reagent articles, such that the articles are highly suited for use in multiplexed assay formats.

### BACKGROUND ART

Multiplexed assay methods have been developed for use in the biotechnology industry and in contemporary laboratory research methods in recent years. Such processes depend for their success on the ability to multiplex parallel processes, assays or reactions, each of which takes place in a similar physical format, in a large collection of essentially identical systems. A common platform for such methods involves use of arrays. An array is typically created on a surface or substrate, divided into a gridwork of array points. Each locus in the array is separately addressable, and carries an identifiable probe for a process or assay, or an identifiable reagent for use in a chemical reaction. Indeed, in certain common arrays, unique probes are constructed at a particular locus by carrying out a unique sequence of chemical reactions in order to provide the desired final product.

A second commonly used modality for multiplexing processes, assays, or reactions employs individual particles or beads as the substrate for the unique probes or reagents. Particles have typically been suspended in a fluid for carrying out an assay, process, or reaction. They have then been segregated from the fluid, typically by gravitational settling, centrifugation, filtration, or via magnetic separation, for removing unneeded or exhausted reaction or assay components, and for washing free of previous reaction or assay compositions.

U.S. Pat. No. 6,579,729 states that in synthesizing combinatorial libraries a variety of forms of encoding have been reported, including binary encoding employing a set of non-sequenceable electrophoric tagging molecules (Ohlmeyer et al. (1993) PNAS 90:10922-10926).

U.S. Pat. No. 6,586,190 relates to a high throughput multiplexed displacement assay which, it reports, incorporates the technology developed by Luminex Corporation, Austin Tex. (U.S. Pat. No. 5,981,180). This technology uses a defined combination of two dyes in 100 different combinations to code beads, which are determined by flow cytometry.

Still according to U.S. Pat. No. 6,586,190, an additional marker system has been referred to as the Quantum Dot™ (Quantum Dot Corporation, Palo Alto, Calif.). It reports that the Quantum Dot™ is a 2-10 nm CdSe crystal which, depending on its size, emits a single wavelength light ranging from ultraviolet to infrared when excited with UV light (Chan and Nie (1998), Science 281:2016-2018). The complexity of the quantum dot population defines the total number of distinct beads that can be encoded.

There are additional known methods and substrate types that can be used for tagging or otherwise uniquely identifying individual beads with attached probes. Known methods include using polystyrene latex spheres that are colored or fluorescently labeled. Other methods include using small plastic cans with a conventional bar code applied, or a small container includes a solid support material and a radio-frequency tag.

There are certain problems or disadvantages encountered with the multiplexed systems described above. Many methods of uniquely identifying the probes may require large structures, have a limited number of identifiable codes, and/or are formed of material not suitable to harsh environmental conditions, such as high temperature and/or corrosive material. In the case of arrays, one can only assay for those substances or components positively bound to a spot or locus in the array. In this sense, an array may be considered a “closed” system in that it is limited to planning or foresight employed in laying out the array. A process, assay, or reaction not conceived of cannot be probed by an array. In many arrays, an effort to create a high density of spots on the substrates imposes spatial limitations on the processes, assays or reactions that may be carried out at each spot. This is because it is difficult to resolve adjacent spots when preparing or using the array. This interferes with the ability of arrays effectively to conduct multiplexed processes

Particles have certain other disadvantages. Although each particle is unique, it may not be distinguishable from its partners without use of some kind of label. Particles may be labeled by dyes, for example, that may provide an analog signal related to coding an identity. Alternatively, a particle may carry a second chemical composition, in addition to the primary composition related to the process, assay, or reaction for which it is intended, that must be identified in order to learn the coding for the particle. Such chemical codes may require “off-line” or secondary processing in order to be identified, removing the versatility of manipulating the particle in “real time”, i.e., within the time frame of a process, assay, or reaction. In addition, certain known bead systems are constrained to a relatively small number of codes available.

From the above discussion it is apparent that there remains a need in multiplexed applications for open systems that are not limited or confined by numerical constraints as to the number of parallel processes, assays, or reactions that may be carried out. There remains a need to provide probes that are very small, capable of providing a large number of unique codes (e.g., greater than 1 million codes), and/or have codes intrinsic to the probe which are resistant to harsh environments. Additionally, there remains a need to move away from array-based systems, since they lack the advantage of versatility of handling and manipulation of the individual positions in the array. In addition, there is a strong need for an article that is encoded so that it can be unambiguously identified in real time in an assay procedure. There is further a need for an assay and article using a code that may be read using physical methods, rather than relying on secondary determinations for reading the code. There is further a need for an assay and article that is conveniently employed in a wide range of biological, chemical, diagnostic and related biotechnological systems for conducting processes, assays, sensing, and reactions. In addition several needs for films, coatings, or membranes disposed on an encoded article exist. The present invention addresses these unmet needs.

### SUMMARY DISCLOSURE OF THE INVENTION

The present invention provides methods and compositions directed toward assays of a broad range of analytes using specific targeting chemicals that bind to the analytes. The assays are founded on the use of coded assay articles to which are attached the targeting chemicals. Additionally the codes are such that they are interrogated and determined during the course of an experiment that also detects and/or quantitates the analyte bound to the article. Since the article is usually fabricated such that the code identifies the particular targeting chemical bound to it, the target is analyzed as to identity, presence and quantity in real time. These attributes facilitate the use of the methods and compositions of the invention in high-complexity multiplexed assay systems.

All the methods and compositions are based on assay article that includes:
i) an optical substrate;
ii) the chemical being bound to the substrate; and
iii) at least a portion of the substrate having at least one diffraction grating disposed therein, the grating having at least one refractive index pitch superimposed at a common location;
wherein the grating provides an output optical signal when illuminated by an incident light signal; and wherein the optical output signal is indicative of a code in the substrate.

In a first aspect the invention provides a method of identifying the presence and/or amount of an analyte, including the steps of:
a) providing an assay article including a chemical bound to an optical identification element, wherein the chemical specifically binds to the analyte and wherein the optical identification element includes the coded optical substrate described above;
b) contacting the assay article with a sample containing the analyte, thereby binding the analyte to the assay article;
c) determining the code provided by the assay article; and
d) determining the presence and/or amount of the analyte bound to the assay article thereby identifying the presence and/or amount of the analyte.

In an additional aspect, the invention provides a method of conducting a multiplexed assay for the presence and/or amount of one or more analytes, including the steps of:
a) providing a plurality of assay articles wherein an assay article includes a chemical bound to an optical identification element, wherein each chemical specifically binds to an analyte, and wherein each optical identification element includes the optical substrate described above;
   wherein the optical output signal is indicative of a first code in the substrate of a first assay article, and the first code differs from a second code provided by a second assay article; and
   wherein a first chemical bound to the substrate of the first assay article is identified by the first code provided thereby, and a second chemical bound to the substrate of the second assay article is identified by the second code provided thereby;
b) contacting the plurality of assay articles with a sample containing one or more analytes, thereby binding an analyte to an assay article to provide a positive assay article;
c) determining the code provided by the positive assay article; and
d) determining the presence and/or amount of an analyte bound to the positive assay article.

In a further aspect, the invention provides a method of identifying the occurrence of a process wherein the process requires an analyte and provides a detectable label bound to an assay article, including the steps of:
a) providing an assay article including a chemical bound to an optical identification element, wherein the chemical binds to the analyte, and wherein the optical identification element includes the optical substrate described in the preceding:
b) contacting the assay article with a sample containing the analyte and a component that permits the process to occur, thereby binding the label to the assay article;
c) determining the code characterizing the assay article; and
d) determining the presence of the label bound to the assay article; thereby identifying the occurrence of the process.

In still an additional aspect the invention provides a method of conducting a multiplexed assay for identifying the occurrence of a process wherein the process requires an analyte and provides a detectable label bound to an assay article, including the steps of:
a) providing a plurality of assay articles wherein an assay article includes a chemical bound to an optical identification element, wherein the chemical binds to the analyte, and wherein the optical identification elements include the optical substrate described in the preceding;
   wherein the optical output signal is indicative of a first code in the substrate of a first assay article, and the first code differs from a second code provided by a second assay article; and
   wherein a first chemical is bound to the substrate of the first assay article and is identified by the first code provided thereby, and a second chemical is bound to the substrate of the second assay article and is identified by the second code provided thereby;
b) contacting the plurality of assay articles with a sample containing the analyte and a component that permits the process to occur, thereby binding a label to at least one assay article;
c) determining the code provided by the at least one assay article; and
d) determining the presence of a label bound to the at least one assay article; thereby identifying the occurrence of the process.

In an advantageous embodiment of the methods of identifying the occurrence of a process, the process labels the chemical, and in an alternative advantageous embodiment the process labels the analyte.

In still an additional aspect the invention provides an assay article including a chemical bound to an optical identification element, the chemical specifically binding to an analyte, wherein the optical identification element includes the optical substrate described above.

In yet a further aspect the invention provides a set including a plurality of assay articles wherein each assay article includes a chemical bound to an optical identification element, the chemical specifically binding to an analyte, wherein each optical identification element includes the optical substrate described above;
wherein the optical output signal is indicative of a first code in the substrate of a first assay article, and the first code differs from a second code provided by a second assay article; and
wherein a first chemical bound to the substrate of the first assay article is identified by the first code provided thereby, and a second chemical bound to the substrate of the second assay article is identified by the second code provided thereby.

In yet an additional aspect the invention provides a method of analyzing a target substance in a sample including:
a) contacting the sample with a plurality of coded assay articles bearing probe substances,
   wherein a probe substance specifically binds a target substance, thereby binding the target substance to a coded assay article,
   wherein each coded assay article includes the probe substance bound to an optical substrate that has been described above;
b) determining the code provided by the assay article; and
c) analyzing the target substance bound to the coded assay article.

Significant embodiments of all the method and composition aspects described above are also provided by the invention. In a significant embodiment the assay article is a particle or bead. In an additional significant embodiment the chemical is bound to the article by a covalent bond. In various covalently bonded embodiments, the chemical includes a nucleic acid, a polynucleotide, an oligonucleotide, a nucleotide, a nucleoside, a protein nucleic acid, an oligopeptide nucleic acid, a protein or fragment thereof, an antibody or fragment thereof, an enzyme or fragment thereof, a receptor or fragment thereof, a polypeptide, an oligopeptide, an amino acid, a derivative of any of them, or a modification of any of them. In further covalently bonded embodiments the chemical includes a moiety chosen from among a synthetic organic molecule, a synthetic intermediate, a synthetic precursor, an antibiotic, a metabolite, a candidate pharmaceutical agent, or a pharmaceutical agent. In still a further embodiment a covalently bonded chemical includes a moiety chosen from among a virus, a prokaryotic cell, a eukaryotic cell, a vertebrate cell, a mammalian cell, a human cell, a subcellular organelle, and a component of any them. In still an additional significant embodiment, the analyte includes a polynucleotide that includes an allele of a single nucleotide polymorphism and the chemical includes a sequence complementary to a sequence including the single nucleotide polymorphism.

In additional significant embodiments of the methods and compositions of the invention a linker is included between the substrate and a moiety including the chemical, and in another significant embodiment the moiety further includes a spacer that binds the moiety to the linker. Still additionally a moiety including the chemical further includes a spacer that binds the moiety to the substrate.

In further advantageous embodiments of the methods and compositions of the invention the chemical is bound to the assay article by noncovalent interactions.

In still additional important embodiments of the methods and compositions provided by the invention the analyte is labeled, and still additionally in an important embodiment the presence and/or amount of the label is determined. In still further important embodiments the label emits radiation and the presence and/or intensity of the radiation is determined.

In additional significant embodiments of the methods provided herein, the step for determining the presence and/or amount of the analyte further includes binding a specific detecting substance to the bound analyte and determining the specific detecting substance; furthermore, in significant embodiments of the latter detecting step the specific detecting substance is labeled.

In still additional important embodiments of the methods and compositions of the invention the substrate includes silica, a silicate, a glass, a semiconducting material, a ceramic material, a polymer, a resin, a rubber material, or a derivative thereof.

In still further aspects the invention provides assay articles and sets of assay articles that are the result of the binding of an analyte to a chemical to form a specific binding pair bound to an article. In their broadest aspects, the invention provides an assay article, or a set of assay articles wherein at least one assay article is bound, that includes a specific binding pair bound to an optical identification element, the specific binding pair including a first specific binding substance bound to a cognate specific binding substance, wherein the optical identification element includes the optical substrate described above. In advantageous embodiments the first specific binding substance is a chemical and the second specific binding substance is an analyte specifically bound by the chemical; in other advantageous embodiments the first specific binding substance is a receptor and the second specific binding substance is a ligand specifically bound by the receptor; and in still further advantageous embodiments the first specific binding substance is a probe and the second specific binding substance is a target specifically bound by the probe.

### BRIEF DESCRIPTION OF THE DRAWINGS

FIG. 1 is a side view of an optical identification element, in accordance with the present invention.

FIG. 2 is a top level optical schematic for reading a code in an optical identification element, in accordance with the present invention.

FIG. 3 is a flow chart of the method of attaching a substance to an optical identification element, performing an assay and analyzing the optical identification element, in accordance with the present invention.

FIG. 4 is a side view of an optical identification element having a substance bound to the outer surface thereof, in accordance with the present invention.

FIG. 5 is a side view of an optical identification element having a substance bound to the outer surface thereof, in accordance with the present invention.

FIG. 6 is a schematic view of a plurality of optical identification elements having different identification or codes and coated with different probe substances disposed in a cell with a plurality of test substances, in accordance with the present invention.

FIG. 7 is a schematic view of plurality of optical identification elements after the performance of an assay, aligned in a plurality of grooves, disposed on a substrate, and a bead detector that scans each optical identification element for determining the code and fluorescence of each optical identification element, in accordance with the present invention.

FIG. 8 is a side view of an optical identification element after the performance of an assay, and a bead detector that determines the code and fluorescence of the optical identification element, in accordance with the present invention.

FIG. 9 is a side view of an optical identification element after the performance of an assay, and a more detailed view of a bead detector that determines the code and fluorescence of the optical identification element, in accordance with the present invention.

FIG. 10 is an optical schematic for reading a code in an optical identification element, in accordance with the present invention.

FIG. 11 is an image of a code on a CCD camera from an optical identification element, in accordance with the present invention.

FIG. 12 is a graph showing an digital representation of bits in a code in an optical identification element, in accordance with the present invention.

FIG. 13 illustrations (a)-(c) show images of digital codes on a CCD camera, in accordance with the present invention.

FIG. 14 illustrations (a)-(d) show graphs of different refractive index pitches and a summation graph, in accordance with the present invention.

FIG. 15 is an alternative optical schematic for reading a code in an optical identification element, in accordance with the present invention.

FIG. 16 illustrations (a)-(b) are graphs of reflection and transmission wavelength spectrum for an optical identification element, in accordance with the present invention.

FIGS. 17-18 are side views of a thin grating for an optical identification element, in accordance with the present invention.

FIG. 19 is a perspective view showing azimuthal multiplexing of a thin grating for an optical identification element, in accordance with the present invention.

FIG. 20 is side view of a blazed grating for an optical identification element, in accordance with the present invention.

FIG. 21 is a graph of a plurality of states for each bit in a code for an optical identification element, in accordance with the present invention.

FIG. 22 is a side view of an optical identification element where light is incident on an end face, in accordance with the present invention.

FIGS. 23-24 are side views of an optical identification element where light is incident on an end face, in accordance with the present invention.

FIG. 25, illustrations (a)-(c) are side views of an optical identification element having a blazed grating, in accordance with the present invention.

FIG. 26 is a side view of an optical identification element having a coating, in accordance with the present invention.

FIG. 27 is a side view of whole and partitioned optical identification element, in accordance with the present invention.

FIG. 28 is a side view of an optical identification element having a grating across an entire dimension, in accordance with the present invention.

FIG. 29, illustrations (a)-(c), are perspective views of alternative embodiments for an optical identification element, in accordance with the present invention.

FIG. 30, illustrations (a)-(b), are perspective views of an optical identification element having multiple grating locations, in accordance with the present invention.

FIG. 31, is a perspective view of an alternative embodiment for an optical identification element, in accordance with the present invention.

FIG. 32 is a view an optical identification element having a plurality of gratings located rotationally around the optical identification element, in accordance with the present invention.

FIG. 33 illustrations (a)-(e) show various geometries of an optical identification element that may have holes therein, in accordance with the present invention.

FIG. 34 illustrations (a)-(c) show various geometries of an optical identification element that may have teeth thereon, in accordance with the present invention.

FIG. 35 illustrations (a)-(c) show various geometries of an optical identification element, in accordance with the present invention.

FIG. 36 is a side view an optical identification element having a reflective coating thereon, in accordance with the present invention.

FIG. 37 illustrations (a)-(b) are side views of an optical identification element polarized along an electric or magnetic field, in accordance with the present invention.

FIG. 38 shows a graph of bead number vs. fluorescence intensity from an assay, in accordance with the present invention.

FIG. 39 shows a graph of Cy5 vs. Cy3 fluorescence intensity from an assay, in accordance with the present invention.

FIG. 40 shows a graph of Cy5 vs. Cy3 fluorescence intensity from an assay, in accordance with the present invention.

FIG. 41 shows a graph of Cy3 fluorescence intensity from a biological assay, in accordance with the present invention.

FIG. 42 shows a representation of fluorescence yield from an experiment in which a pool of 81 different coded beads was used to probe for a target.

FIG. 43 shows a dose titration of the fluorescence response for three labeled targets.

FIG. 44 shows the kinetics of hybridized probe-target complexes on particles of the invention in various hybridization buffers.

FIG. 45 shows a comparison of assays conducted using particles of the present invention (left panel) and comparable assays performed using a commercial microarray (right panel).

FIG. 46 shows detection of rabbit beta globin transcript when it was added as a supplement to a library of RNA molecules.

FIG. 47 is a schematic representation of a direct immunoassay using a secondary antibody that is labeled.

FIG. 48 is a schematic representation of an indirect immunoassay using a secondary antibody that is detected by an additional reagent.

FIG. 49 presents an immunoassay for TNF-a. Left panel, a schematic representation of the final detected complex used in the immunoassay. Right panel, graph showing fluorescence results for a duplexed immunoassay using a secondary antibody and indirect labeling.

FIG. 50 shows a determination of the sensitivity of an immunoassay for TNF-alpha.

FIG. 51 shows a titration of signal intensity from various concentrations of TNF-a.

FIG. 52 shows results depicting the specificity of an immunoassay for TNF-a.

FIG. 53 presents a schematic diagram of SNP detection by allele specific hybridization.

FIG. 54 presents a schematic diagram of SNP detection by single base chain extension.

FIG. 55 presents a schematic diagram of SNP detection by allele specific primer extension.

FIG. 56 presents a schematic diagram of SNP detection by oligonucleotide ligation assay.

FIG. 57 presents a schematic diagram of SNP detection by allele specific PCR.

FIG. 58 shows the sensitivity of a react-and-combine protocol for detection of particles.

### MODES FOR CARRYING OUT THE INVENTION

In its most general aspect the present invention provides a diffraction grating-based encoded element wherein the element includes an optical substrate and a surface, at least a portion of the surface thereof having a chemical bonded thereto. The nature of the chemical broadly encompasses a molecular, supramolecular, polymeric, resinous, plastic or rubber structure bound to at least a portion of the surface. The chemical accomplishes a broad range of intended objectives such as carrying out processes, assays, sensing, and reactions; and so forth. In various embodiments of the present invention the chemical includes a reagent.

As used herein including the claims, the indefinite articles “a” and “an”, and the definite article “the”, when modifying a noun, refer to the noun in both the singular and the plural. Thus the phrase “a substance” may mean both a single substance and a plurality of substances.

As used herein “a diffraction grating-based encoded element having a chemical bonded thereto” and similar terms and phrases relates to any construct of the invention including an optically encoded diffraction grating for identification of the element and a substance or material adhered or bonded thereto. As used herein, the phrase “a diffraction grating-based encoded element having a chemical bonded thereto”, and similar terms and phrases, may be abbreviated to or substituted by “assay article”, “multicomponent article”, “reagent article”, and similar terms and phrases. The phrase “a diffraction grating-based encoded element” may be substituted herein by similar terms and phrases, including by way of nonlimiting example “optically encoded element”, “grating encoded element”, “optical element”, and so forth. The terms “diffraction grating-based encoded micro-particle”, “diffraction grating-based encoded element”, and “optical identification element” have been used in related, co-owned U.S. patent applications, including U.S. Ser. No. 10,645,686 filed Aug. 20, 2003, and U.S. Ser. No. [CyVera Docket No. CV-0076/409-01] filed Aug. 18, 2004, to describe identical or similar objects.

The optically encoded element is broadly understood as having no prescribed size or shape. Its size as measured by a largest dimension thereof may range from as large as 1 mm, or as large as 1 m, or even larger, to as small as 1 μm, or as small as 1 nm, or even smaller. Its shape is provided so as to carry out a particular function or purpose in optimal fashion.

In advantageous embodiments a diffraction grating-based encoded element is fabricated as a particle. Accordingly, the present invention provides a diffraction grating-based encoded particle to which is bound one or more substances for carrying out an unrestricted variety of physical, chemical or biological processes, assays, sensing, or reactions. The phrase “diffraction grating-based encoded particle” may be substituted herein by similar terms and phrases, including by way of nonlimiting example “optically encoded particle”, “grating encoded particle”, “optical particle”, “assay particle”, “reagent particle”, and so forth. Because the number of particles employed in any one determination is in principle without limit, the particles are eminently suitable for use in multiplexed processes, i.e., in high throughput systems. The particles carry an embedded code, which in advantageous embodiments is a digital code, and which is rapidly readable by optical instrumentation so that the identity of the particle is immediately available, even if its physical location in an particle is random. Likewise, there is a one-to-one correspondence between the embedded code and the identity of the substance that the particle carries; in other embodiments the identity of the substance is determined, thus establishing the code-substance correspondence. The particles are inexpensive to manufacture and the identification codes are easy and inexpensive to imprint into the particles. In this regard, since each particle is encoded, its route through a microfluidic system, such as a flow sorter, is readily controllable by programmable fluid switches or flow controls. Establishment of a code-substance correspondence permits the particles of the invention to be used in any of a broad range of processes, assays, sensing, and reactions. The substances bound to the particles may be, without limitation, any biological macromolecule or fragment thereof, or any biological metabolite, or any low molecular weight compound. These may be screened in high throughput systems for ability to bind, react with, or identify a target substance in a sample. Substances bound to particles of the invention may be also serve as intermediates in a synthetic reaction scheme to synthesize a desired substance in situ, bound to the particles. The above discussion identifies exemplary uses for the particles of the invention, without intending to limit such uses in any way.

As used herein the terms “diffraction grating-based encoded particle”, “particle”, “assay particle”, “bead”, “microbead”, “assay bead”, and similar terms and phrases are used synonymously to designate a relatively small construct whose size is adequate both to contain upon it or within it a code readable by a suitable device, and to have bound to it sufficient chemical material to serve the functions and purposes of the invention. In favorable embodiments the code is a digital code. Thus without limiting the scope of the invention, a particle of the invention may range in a longest dimension from as small as a fraction of a micrometer or smaller to as large as 1 millimeter or larger. Attributes of a suitable particle include ease of handling in various laboratory and assay formats, ease of applying or embedding a code, ease of binding or attaching a reagent, and ease of determining both the embedded code and the attributes of the reagent. A further attribute of certain embodiments of a particle of the invention is its ease of handling in microfluidic flow systems. In view of the above considerations, the overall shape of a particle of the invention is not circumscribed or limited by any description herein, but rather a particle may be fabricated optimally to accomplish objectives such as those mentioned above. Likewise details of the shape, cross section, and other descriptions of the three-dimensional geometry of a particle are not limited by any description herein. In general, any equivalent of a particular particle described herein is intended to fall within the scope of the claims.

As used herein the terms “substrate”, “optical substrate”, and similar terms and phrases relate to at least a major component, if not the entire component, that constitutes an optically encoded element employed in the invention. The substrate has applied to or embedded within its structure a code that provides the coding for the article. In many embodiments of the invention an instrumental reader employing optical radiation is used to read the code, including a digital code (see below). In those cases the substrate is an “optical substrate” as used herein, having optical transparency or analogous attributes that adapt it for reading the code in the practice of the invention. Any of several functionally equivalent materials or compositions may be employed to provide a substrate or an optical substrate of the present invention.

As used herein the terms “code”, “encoded” and similar terms and phrases are broadly intended to relate to a readable code applied on or embedded within an article of the invention such that a given article is identifiable by its code. Advantageously the code is readable in “real time”, i.e., in the time during which an experimental measurement of a property of the article is being made. The code is comprised of one or more positions in a series of positions, wherein each position in the code bears a permitted value for the code being employed. In many important embodiments disclosed herein the code is a digital code, wherein each position of the code assumes only allowed discrete values. If the code is binary (base 2), one of two values occurs at each position; likewise if the code has base n, the value at a particular position in the series is one of the n discrete values that characterizes the base n code. The series of positions in the code is readable by suitable instrumentation employed in the practice of the invention, thereby providing the complete code that identifies the article; advantageously the code is readable in “real time” while a article is being employed in a process, assay, or reaction. In general, any equivalent of a particular code system described herein is intended to fall within the designation of a code of the invention, including any digital code, and to fall within the scope of the claims.

As used herein the term “optical coding element” and similar terms and phrases relates to a series of encoded positions applied to or embedded within a substrate. An optical coding element is readable by instrumentation employing, by way of nonlimiting example, any instrument or reader capable of interrogating the code. An example of such a reader is disclosed in copending application U.S. Ser. No. xxxxxx (CyVera Docket No. CV-0026PR). The series of positions in the optical coding element define a code for the article on which or in which the element appears. Any equivalent encoded optical coding element, including a digital optical coding element, is encompassed within the scope of the present invention.

As used herein the terms “substance” or “material” and similar terms and phrases relate broadly to any material entity bound to at least a portion of the surface of the diffraction grating-based encoded element to provide a multicomponent article of the invention. The substance or material may be bound to a surface of the article by adhesion or adherence, including any noncovalent interaction. Alternatively the substance or material may be bound covalently to reactive groups included on the surface of the optically encoded element.

As used herein the terms “chemical”, “reagent”, and similar terms and phrases are employed broadly to designate a chemical substance that is a substance of interest in the invention, and that is bound or coupled to an optically encoded element to form a multicomponent article of the invention. The terms “chemical” and “reagent” may be used herein synonymously with the terms “reactant”, “ligand”, “probe”, “active agent”, and related terms and phrases. A particular usage may depend on a particular context. In general, a particular chemical bound or coupled to a an optically encoded element accomplishes a particular objective of the invention in a process, assay or reaction in which the reagent takes part. Any reagent of the invention is one of the two members of a “specific binding pair” or a “specific reactant pair”. In certain circumstances more than two reagents engage in a specific binding interaction or a specific reactant process, in which case the synonymous designations “specific binding set” or “specific reactant set” may be employed.

The members of a specific binding pair or a specific binding set may interact by noncovalent interactions only; specificity is determined by the spatial distribution and nature of the noncovalent interactions determining the binding process. In such cases a reagent may synonymously be designated a “ligand” or a “probe” herein. The cognate member(s) of the binding pair or binding set may then be designated by terms such as “receptor”, or “target”, respectively, and similar terms and phrases known to workers of skill in fields related to the present invention. Thus, when bound, a ligand-receptor pair, or a probe-target pair, is formed. In general, when a chemical probe is bound to an optically encoded element, any specific target that is a cognate of the probe that is present in a composition in which the encoded element is suspended may bind to the probe; and likewise for a bound ligand and its cognate receptor.

The reagent of a multicomponent article may also be a reactant employed in a chemical synthesis to create a new chemical substance by reaction with one or more cognate reactants. The cognates are contained within a composition in which the reagent article is suspended. In this case the specific reactant pair or the specific reactant set combine by forming new covalent bonds to generate the new chemical substance as the product of the reaction.

In general the designations “reagent”, “reactant”, “ligand”, “probe”, “active agent”, and related terms and phrases is understood by workers of skill in fields related to the present invention to encompass the full breadth of chemical substances to be bound in a multicomponent article of the invention without limitation.

As used herein the terms “moiety”, “radical”, “fragment”, “grouping”, and similar terms and phrases, are synonymously related to a chemical component that is a portion or a fragment of a larger chemical entity or chemical compound. In general a moiety, radical, or grouping has at least one free chemical bond. The free chemical bond binds the moiety, radical, or grouping to a cognate portion of the larger chemical element.

As used herein the term “linker” and similar terms is related to a chemical moiety interposed between the surface of an article, such as a reagent particle, and a reagent-bearing moiety. In general a linker moiety links a particle to a reagent-bearing moiety. Thus in general the linker precursor used to incorporate the linker into the reagent particle of the invention is at least bifunctional, and may have a functionality of 3 or greater. In addition a linker may serve additional functions such as extending the reagent away from close proximity to the surface of the particle to permit ease of binding or reaction of the reagent to its cognate binding member(s). The chemical description of certain embodiments of a linker is provided below. In general any equivalent moiety serving to adapt the reagent-bearing moiety to the particle surface is considered within the scope of the present invention.

As used herein the term “spacer” and similar terms is related to a fragment of a reagent-bearing moiety that serves to bind the reagent to the linker. The properties of a spacer are similar to those of a linker, but as used herein the two terms are distinguished as defined in these paragraphs and elsewhere in this specification. Thus a spacer precursor is likewise at least bifunctional and may have a functionality of 3 or higher. A spacer precursor is designed to form a covalent bond with the linker, on one hand, and with the reagent on the other. In general any equivalent moiety serving to adapt the reagent to the linker is considered within the scope of the present invention.

As used herein the term “heteroatom” relates to a divalent O or S atom, or to a divalent NR grouping, wherein R may be H, normal or branched chain alkyl, normal or branched chain alkylene, cycloalkyl, aryl, normal or branched chain alkoxy, cycloalkoxy, aryloxy, normal or branched chain alkylamino, normal or branched chain alkyleneamino, cycloalkylamino or arylamino.

A Diffraction Grating-Based Encoded Element

An optically encoded element of the invention is constituted at least in a significant portion thereof, if not entirely, of an optical substrate, also termed a substrate. In important embodiments the substrate is an optical substrate. In important examples the substrate is constructed of a silica or a silicate glass material. Silica or silicates have Si—O⁻ or Si—OH groupings on the surface, which may be utilized as a reactive grouping for binding a linker. A variety of reagents for binding to silica or silicate glasses is available from Gelest, Inc. (Morrisville, Pa.) as well as from other vendors. Certain modalities for derivatizing substrates such as silica are presented in U.S. Pat. Nos. 6,444,268 B2 and 6,3219,674 B1.

Alternatively the substrate may be constituted of a polymer or resin. The polymer or resin may adsorb a reagent by noncovalent interaction, or it may have, or be derivatized to bear, substituent groups on the surface of the substrate to which a linker may be bound. Nonlimiting examples of polymers useful in preparing bead substrates include homopolymers and copolymers of polystyrene and derivatives thereof, polyamides such as various nylons, polyvinyl alcohol resins, polyacrylates (including esters and crosslinked resins thereof), polymethacrylates (including esters and crosslinked resins thereof), polyacrylamides including crosslinked resins thereof, polycarbonates, polyesters including polycolactide-glycolides, latexes, and several other polymers, and resins known in the art. Many polymers and resins are known as supports in various solid phase assays, processes and synthetic reactions. A general set of definitions of various categories of polymers useful as optical substrates of the invention is given in Pure Appl. Chem., Vol. 68, No. 12, pp. 2287-2311, 1996.

As an example, U.S. Pat. No. 6,607,921 states that representative supports for various bound reagents include, by way of illustration, polymeric (resin) beads, polymeric gels, glass beads, silica chips and capillaries, agarose, diatomaceous earths, pulp, and the like. The patent identifies preferred solid as those having minimal non-specific binding properties, and further as derivatized porous polystyrene-divinylbenzene polymer beads, such as POROS beads (available from Perseptive Biosystems, Framingham, Mass.).

Furthermore, an optical substrate may have a compounded structure composed of more than one substance or material. As a nonlimiting example, a diffraction grating-based encoded element may have an inner component composed of one substance, and be coated with a different substance. Thus, an inner component may be made of silica or a glass, and may be coated with a polymer material. In the case of complex structures, a surface for binding a reagent is an outermost surface.

A diffraction grating-based encoded element provided by the present invention generally includes an optical substrate having at least one surface. The optical substrate includes an optical coding element providing an output signal corresponding to a code, such as a digital code, embedded therein when the coding element is illuminated with incident radiation. In significant embodiments of the invention the optical coding element comprises an optical diffraction grating. In addition, the reagent-bearing article, such as a reagent particle, includes a reagent bound to a surface of the substrate.

As noted above, common embodiments of the invention provide an element in the form of a particle and the substance is a reagent bound to the particle. Advantageously the reagent is bound via a linker interposed between a surface of the particle and a reagent moiety that includes the reagent as part of its structure. Additionally the reagent moiety may include a spacer placed between the linker and the reagent.

Reagent Particle with Optical Substrate and Grating

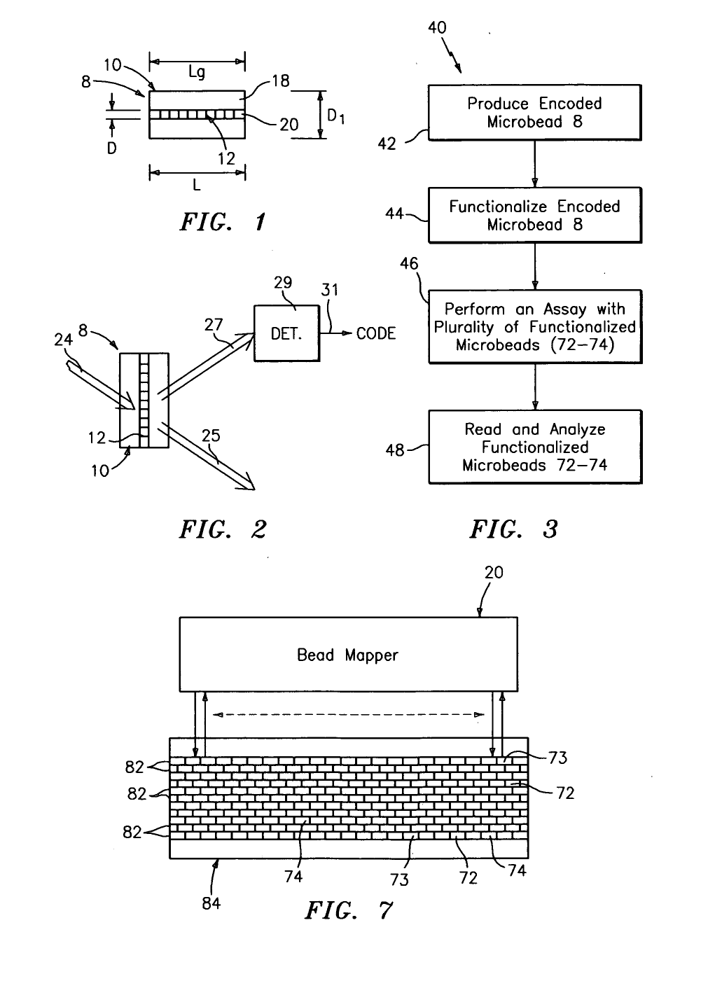

An important embodiment of a reagent particle of the invention is represented in FIG. 1. A diffraction grating-based optical identification element 8 (or encoded element or coded element) comprises a known optical substrate 10, having an optical diffraction grating 12 disposed (or written, impressed, embedded, imprinted, etched, grown, deposited or otherwise formed) in the volume of or on a surface of a substrate 10. The grating 12 is a periodic or aperiodic variation in the effective refractive index and/or effective optical absorption of at least a portion of the substrate 10.

The optical identification element 8 described herein is the same as that described in Copending Patent Application Serial No. (CiDRA Docket No. CC-0648A), filed contemporaneously herewith, which is incorporated herein by reference in its entirety.

In particular, the substrate 10 has an inner region 20 where the grating 12 is located. The inner region 20 may be photosensitive to allow the writing or impressing of the grating 12. The substrate 10 has an outer region 18, which does not have the grating 12 therein.

The grating 12 is a combination of one or more individual spatial periodic sinusoidal variations (or components) in the refractive index that are collocated at substantially the same location on the substrate 10 along the length of the grating region 20, each having a spatial period (or pitch) A. The resultant combination of these individual pitches is the grating 12, comprising spatial periods (Λ1-Λn) each representing a bit in the code. Thus, the grating 12 represents a unique optically readable code, made up of bits, where a bit corresponds to a unique pitch Λ within the grating 12. Accordingly, for a digital binary (0-1) code, the code is determined by which spatial periods (Λ1-Λn) exist (or do not exist) in a given composite grating 12. The code or bits may also be determined by additional parameters (or additional degrees of multiplexing), and other numerical bases for the code may be used, as discussed herein and/or in the aforementioned patent application.

The grating 12 may also be referred to herein as a composite or collocated grating. Also, the grating 12 may be referred to as a “hologram”, as the grating 12 transforms, translates, or filters an input optical signal to a predetermined desired optical output pattern or signal.

The substrate 10 has an outer diameter D1 and comprises silica glass (SiO₂) having the appropriate chemical composition to allow the grating 12 to be disposed therein or thereon. Other materials for the optical substrate 10 may be used if desired. For example, the substrate 10 may be made of any glass, e.g., silica, phosphate glass, borosilicate glass, or other glasses, or made of glass and plastic, or solely plastic. For high temperature or harsh chemical applications, the optical substrate 10 made of a glass material is desirable. If a flexible substrate is needed, plastic, rubber or polymer-based substrate may be used. The optical substrate 10 may be any material capable of having the grating 12 disposed in the grating region 20 and that allows light to pass through it to allow the code to be optically read.

The optical substrate 10 with the grating 12 has a length L and an outer diameter D1, and the inner region 20 diameter D. The length L can range from very small “microbeads” (or microelements, micro-particles, or encoded particles), about 1-1000 microns or smaller, to larger “macroelements” for larger applications (about 1.0-1000 mm or greater). In addition, the outer dimension D1 can range from small (less than 1000 microns) to large (1.0-1000 mm and greater). Other dimensions and lengths for the substrate 10 and the grating 12 may be used.

The grating 12 may have a length Lg of about the length L of the substrate 10. Alternatively, the length Lg of the grating 12 may be shorter than the total length L of the substrate 10.

The outer region 18 is made of pure silica (SiO₂) and has a refractive index n2 of about 1.458 (at a wavelength of about 1553 nm), and the inner grating region 20 of the substrate 10 has dopants, such as germanium and/or boron, to provide a refractive index n1 of about 1.453, which is less than that of outer region 18 by about 0.005. Other indices of refraction n1,n2 for the grating region 20 and the outer region 18, respectively, may be used, if desired, provided the grating 12 can be impressed in the desired grating region 20. For example, the grating region 20 may have an index of refraction that is larger than that of the outer region 18 or grating region 20 may have the same index of refraction as the outer region 18 if desired.

Referring to FIG. 2, an incident light 24 of a wavelength λ, e.g., 532 nm from a known frequency doubled Nd:YAG laser or 632 nm from a known Helium-Neon laser, is incident on the grating 12 in the substrate 10. Any other input wavelength λ can be used if desired provided λ is within the optical transmission range of the substrate (discussed more herein and/or in the aforementioned patent application). A portion of the input light 24 passes straight through the grating 12, as indicated by a line 25. The remainder of the input light 24 is reflected by the grating 12, as indicated by a line 27 and provided to a detector 29. The output light 27 may be a plurality of beams, each having the same wavelength λ as the input wavelength λ and each having a different output angle indicative of the pitches (Λ1-Λn) existing in the grating 12. Alternatively, the input light 24 may be a plurality of wavelengths and the output light 27 may have a plurality of wavelengths indicative of the pitches (Λ1-Λn) existing in the grating 12. Alternatively, the output light may be a combination of wavelengths and output angles. The above techniques are discussed in more detail herein and/or in the aforementioned patent application.

The detector 29 has the necessary optics, electronics, software and/or firmware to perform the functions described herein. In particular, the detector reads the optical signal 27 diffracted or reflected from the grating 12 and determines the code based on the pitches present or the optical pattern, as discussed more herein or in the aforementioned patent application. An output signal indicative of the code is provided on a line 31.

As used herein, the terms “bound”, “attached” and similar terms relate to both noncovalent and covalent association of a chemical or reagent with a substrate of an assay article.

Optically Encoded Multicomponent Article with Adsorbed Reagent

A chemical may be bound by adsorption, i.e., by noncovalent interactions, to an optical substrate. A variety of reagents may be adsorbed in this way. Many proteins and polypeptides are sufficiently surface active that they adhere strongly to a surface of an optical substrate. Included in this category are antibodies. In addition, nucleic acids and polynucleotides may bind noncovalently to a surface of an optical substrate. One embodiment believed, without wishing to bound by theory, to involve electrostatic interactions is represented as a polycation that is first adsorbed to a surface, such as a surface of an optical substrate comprised of silica or a silicate. Silica or a silicate is believed to manifest negative fixed charges on the surface. A polycation commonly used is poly-(L-lysine); other examples include polyethylenimine and polyvinylamine. Subsequently a nucleic acid, polynucleotide or oligonucleotide, which is a polyanion is then bound to the polycation, providing a reagent-bearing article with a nucleic acid, polynucleotide or oligonucleotide bound as the reagent.

An alternative embodiment of an adsorbed reagent thought to be bound by electrostatic interactions involves constructing an optical substrate covalently bound to a linker (see below) that terminates in a cationic group. A high density of fixed positive charges on the surface of the substrate results. These then may adsorb a polyanion such as a nucleic acid, polynucleotide or oligonucleotide, as in the preceding paragraph.

Alternatively a substrate may be coated with a substance that is primarily nonpolar or hydrophobic in nature. Examples include long chain fatty acids, fatty acid esters, phospholipids, other amphiphiles, waxes, hydrophobic polymers, and the like. With a surface on the substrate now having a nonpolar or hydrophobic character, a reagent with an ability to adsorb to such a surface may be applied. Many proteins adsorb to such nonpolar surfaces while still preserving their biological function. Single strand oligonucleotides and polynucleotides may also bind to such a coated surface.

In certain embodiments, a substance modeling a biological lipid bilayer may be constructed. Then a reagent such as a protein that occurs naturally embedded within a biological lipid bilayer membrane may be adsorbed to the optical substrate via the bilayer membrane. In order to accomplish this, a substrate may be coated with a mock or artificially induced lipid bilayer. By way of nonlimiting example, a cationic phospholipid, such as a phosphatidyl choline or a phosphatidyl serine may first be adsorbed, wherein the cationic charge in the polar head of the amphiphile adsorbs to a negative surface charge of an article such as a silica or a silicate. A second layer of an amphiphilic lipid adsorbs to the first layer to result in a lipid bilayer construct that resembles a natural biological membrane. A holoprotein membrane-bound protein including a hydrophobic membrane anchor, or a covalently bound fatty acyl anchor, is then adsorbed to the lipid bilayer, resulting in an article bearing the membrane protein as a reagent.

In more general embodiments of a membrane-coated article, any amphiphile that forms a micelle, or that is employable in the formation of a liposome structure, may be adsorbed to a substrate as described in the preceding paragraph to form a substrate coated with a lipid bilayer. Similarly, in nonaqueous, or nonpolar, solvents, a single layered membrane of amphiphiles may be bound.

In general, any equivalent coating of a substrate to provide an article with a surface different from that of the uncoated substrate and characteristic of the material used in the coating is contemplated within the scope of the invention. Such coatings are known to workers of skill in fields related to the present invention. After acquiring a coating, any reagent contemplated within the scope of the invention may be adsorbed to the surface presented by the coating.

Optically Encoded Multicomponent Article with Covalently Bound Chemical or Reagent

A chemical may be bound to a substrate of an assay article covalently. In general a moiety including the chemical or reagent may be bound via an optional spacer to an optional linker, in the attachment of the reagent-bearing moiety to the substrate. The reagent, if neither a spacer nor a linker is present, or a spacer, if no linker is present, or a linker if present, is bound to the substrate of the article. Appropriately chosen bifunctional spacers and linkers and/or reactive groups are used to bring about the covalent bonding of the chemical to the substrate. Modes of binding a chemical or reagent to a substrate are disclosed in detailed in co-owned U.S. Provisional Application Ser. No. [CyVera Docket No. CV-0076/409-01] filed Aug. 18, 2004. Certain nonlimiting embodiments describing noncovalent and covalent binding of a chemical to a substrate are found in the Examples.

The Chemical or Reagent

The reagent may be any chemical substance useful in a process, assay, or reaction to which the reagent-bearing article of the invention may be applied. In many important embodiments of the invention, a reagent may include a nucleic acid, a polynucleotide, an oligonucleotide, a nucleotide, a nucleoside, a protein nucleic acid, a peptide nucleic acid, a protein or fragment thereof, an antibody or fragment thereof, an enzyme or fragment thereof, a receptor or fragment thereof, a polypeptide, an oligopeptide, an amino acid, a derivative of, any of the foregoing, a modification of any of the foregoing, a synthetic organic molecule, a synthetic intermediate, a synthetic precursor, an antibiotic, a metabolite, any biochemical moiety, a candidate pharmaceutical agent, or a pharmaceutical agent.

Polynucleotides

As used herein the terms “nucleic acid” and “polynucleotide” are considered synonymous with each other, and are used as conventionally understood by workers of skill in fields such as biochemistry, molecular biology, genomics, and similar fields related to the field of the invention. A polynucleotide employed in the invention may be single stranded or a base paired double stranded structure, or even a triple stranded base paired structure. A polynucleotide may be a DNA, an RNA, or any mixture or combination of a DNA strand and an RNA strand, such as, by way of nonlimiting example, a DNA-RNA duplex structure. A polynucleotide and “oligonucleotide” as used herein are identical in any and all attributes defined here for a polynucleotide except for the length of a strand. As used herein, a polynucleotide may be about 50 nucleotides or base pairs in length or longer, or about 60, or about 70, or about 80, or about 100, or about 150, or about 200, or about 300 nucleotides or base pairs or even longer. An oligonucleotide may be at least 3 nucleotides or base pairs in length, and may be shorter than about 70, or about 60, or about 50, or about 40, or about 30, or about 20, or about 15 nucleotides or base pairs in length.

A “nucleoside” is conventionally understood by workers of skill in fields such as biochemistry, molecular biology, genomics, and similar fields related to the field of the invention as comprising a monosaccharide linked in glycosidic linkage to a purine or pyrimidine base; and a “nucleotide” comprises a nucleoside with at least one phosphate group appended, typically at a 3′ or a 5′ position (for pentoses) of the saccharide, but may be at other positions of the saccharide. Nucleotide residues occupy sequential positions in an oligonucleotide or a polynucleotide. Accordingly a modification or derivative of a nucleotide may occur at any sequential position in an oligonucleotide or a polynucleotide. All modified or derivatized oligonucleotides and polynucleotides are encompassed within the invention and fall within the scope of the claims. Modifications or derivatives can occur in the phosphate group, the monosaccharide or the base.

By way of nonlimiting examples, the following descriptions provide certain modified or derivatized nucleotides. The phosphate group may be modified to a thiophosphate or a phosphonate. The phosphate may also be derivatized to include an additional esterified group to form a triester. The monosaccharide may be modified by being, for example, a pentose or a hexose other than a ribose or a deoxyribose. The monosaccharide may also be modified by substituting hydryoxyl groups with hydro, halo, or amino groups, by esterifying pendant hydroxyl groups, by converting a hydroxyl group to an ether, and so on.

The base may be modified in many ways; several modified bases occur naturally in various nucleic acids, and other modifications may mimic or resemble such naturally occurring modified bases. Nonlimiting examples of modified or derivatized bases include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Nucleotides may also be modified to harbor a label. Nucleotides bearing a fluorescent label or a biotin label, for example, are available from Sigma (St. Louis, Mo.).

Any modified nucleotide equivalent to a nucleotide described herein may be part of a polynucleotide reagent bound to an assay article. Such equivalents are known to workers of skill in fields related to the present invention.

A significant use of a nucleic acid-, polynucleotide-, or oligonucleotide-bearing article is in an assay directed to identifying a target sequene to which the probe hybridizes. The selectivity of a probe for a target is affected by the stringency of the hybridizing conditions. “Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

Nonlimiting examples of “stringent conditions” or “high stringency conditions”, as defined herein, include those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

High stringency conditions promote high selectivity in the hybridization of a probe to a target. Stringency conditions may be modified or adjusted by a worker of skill in the art to adapt hybridization conditions to use in high throughput or multiplexed assay systems (Ausubel et al.). In addition, in high throughput or multiplexed assay systems, both the probe characteristics and the stringency may be optimized to permit achieving the objectives of the multiplexed assay under a single set of stringency conditions.

Protein Nucleic Acids

As used herein, the terms “protein nucleic acids”, “peptide nucleic acids”, or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675. A PNA can be incorporated into a reagent-bearing article as the reagent to serve as a probe in a diagnostic assay.

Polypeptides and Proteins

As used herein an “amino acid” designates any one of the naturally occurring alpha-amino acids that are found in proteins. In addition, the term “amino acid” designates any nonmaturally occurring amino acids known to workers of skill in protein chemistry, biochemistry, and other fields related to the present invention. These include, by way of nonlimiting example, sarcosine, hydroxyproline, norleucine, alloisoleucine, cyclohexylalanine, phenylglycine, homocysteine, dihydroxyphenylalanine, ornithine, citrulline, D-amino acid isomers of naturally occurring L-amino acids, and others. In addition an amino acid may be modified or derivatized, for example by coupling the side chain with a label. Any amino acid known to a worker of skill in the art may be used as a reagent in a reagent-bearing article of the present invention.

Amino acid residues are constituents of oligopeptides, polypeptides and proteins. As used herein an “oligopeptide” or “peptide” may be at least 3 amino acid residues in length, and may be shorter than about 70, or about 60, or about 50, or about 40, or about 30, or about 20, or about 15, or about 10 amino acid residues in length. Many peptides are of direct interest, since a number of biologically active substances are relatively short peptides. In addition, amino acid sequences identified as serving as motifs or domains are relatively short. Thus an oligopeptide or polypeptide bound to an assay article may be a fragment of a holoprotein; frequently such fragments retain a biological function characteristic of a domain or a holoprotein from which it is derived. These and a wide range of other peptides or oligopeptides known to workers of skill in fields related to the present invention may serve as reagents in a reagent-bearing article of the invention.

As used herein a “polypeptide” or a “protein” may be considered to have a chain length of at least 50 amino acid residues, and may have as many as about 100, or about 150, or about 200, or about 300, or about 400, or about 500, or about 700, or about 1000 or more amino acids in the molecule. A protein is generally considered to be a composition that occurs naturally and may be isolated from a natural source. As such a protein may also have other characteristics. By way of nonlimiting example, a protein may additionally be a complex between two or more individual polypeptide chains held together by noncovalent interactions and/or by covalent bonds. A protein may additionally be a mature form of a polypeptide chain that is the gene product of an mRNA arising from a gene.

As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

A protein, polypeptide, oligopeptide or peptide may be modified by introducing one or more amino acid substitutions such that the amino acid sequence of the resulting product differs from the sequence that occurs in the naturally occurring substance.

Proteins have a wide range of functions and activities in biological organisms. Important examples of proteins include, by way of nonlimiting example, enzymes, receptors, and antibodies. Enzymes are reagents of interest in the present invention, since certain enzymes may be implicated, for example, in various pathological conditions. In such cases, it may be of interest to detect the presence of a substrate in a target sample, or to identify inhibitors from a set of candidates in a target sample. Likewise a receptor may be a reagent of interest, since binding of a specific ligand to a receptor as an agonist typically induces a signaling cascade leading to downstream sequellae in a cell. Many pathological states result from inappropriate receptor signaling. A receptor as a reagent bound to a particle may also be used to identify a therapeutic antagonist in a target composition to which it is exposed.

A reagent of the present invention may be any one of an amino acid, a peptide, an oligopeptide, a polypeptide, a protein, a receptor, an enzyme, or an antibody.

Antibodies

An antibody may be used as a probe to detect its cognate antigen in a target composition. Thus an antibody and its cognate antigen form a specific binding pair. For this reason antibodies are also an important class of reagent in a reagent-bearing article of the present invention. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}′ and F_{(ab′)2} fragments, and an Fab expression library. In general, antibody molecules obtained from humans relates to any of the classes IgG, IgM, IgA, IgE and IgD, which differ from one another by the nature of the heavy chain present in the molecule. Certain classes have subclasses as well, such as IgG1, Ig.2, and others. Furthermore, in humans, the light chain may be a kappa chain or a lambda chain. Reference herein to antibodies includes a reference to all such classes, subclasses and types of human antibody species. Any antibody disclosed herein binds “immunospecifically” to its cognate antigen. By immunospecific binding is meant that an antibody raised by challenging a host with a particular immunogen binds to a molecule such as an antigen that includes the immunogenic moiety with a high affinity, and binds with only a weak affinity or not at all to non-immunogen-containing molecules. As used in this definition, high affinity means having a dissociation constant less than about 1 μM, and weak affinity means having a dissociation constant higher than about 1 μM.

An isolated protein that is a target of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, antigenic peptide fragments of the antigen may be used as immunogens. An antigenic peptide fragment comprises at least 6, or at least 10, or at least 15 amino acid residues of the amino acid sequence of the full length protein, and encompasses an epitope thereof such that an antibody raised against the peptide forms a specific immune complex with the full length protein or with any fragment that contains the epitope. Antibodies that are specific for one or more domains within an antigenic protein, or derivatives, fragments, analogs or homologs thereof, may also be used as a reagent of the invention. A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988; Cold Spring Harbor Laboratory Press, Cold Spring Harbor, N.Y., incorporated herein by reference). Some of these antibodies are discussed below.

1. Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing, or by a conjugate with a second protein known to be immunogenic in the mammal being immunized.

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum, or with immunoaffinity chromatography.

2. Monoclonal Antibodies

The term “monoclonal antibody” (MAb) as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975 (See also Goding, Monoclonal Antibodies: Principles and Practice, Academic Press, (1986) pp. 59-103)). The monoclonal antibodies can also be made by recombinant DNA methods, such as those described in U.S. Pat. No. 4,816,567.

3. Humanized Antibodies

The antibody reagents can further comprise humanized antibodies or human antibodies. Humanized forms of antibodies are chimeric immunoglobulins, immunoglobulin chains or fragments thereof (such as Fv, Fab, Fab′, F(ab′)2 or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al, Nature 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.)

4. Human Antibodies

Fully human antibodies relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al. (1983) Immunol Today 4: 72) and the EBV hybridoma technique to produce human monoclonal antibodies (see Cole, et al. (1985) In: MONOCLONAL ANTIBODIES AND CANCER THERAPY, Alan R. Liss, inc., pp. 77-96). Human monoclonal antibodies may be utilized in the practice of the present invention and may be produced by using human hybridomas (see Cote, et al. (1983) Proc Natl Acad Sci USA 80: 2026-2030) or by transforming human B-cells with Epstein Barr Virus in vitro (see Cole, et al. (1985) in: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter (1991) J. Mol. Biol., 227:381; Marks et al. (1991) J. Mol. Biol., 222:581). Similarly, human antibodies can be made by introducing human immunoglobulin loci into transgenic animals. This approach is described, for example, in U.S. Pat. Nos. 5,545,807; 5,545,806; 5,569,825; 5,625,126; 5,633,425; 5,661,016, and in Marks et al. (1992) (Bio/Technology 10, 779-783); Lonberg et al. ((1994) Nature 368 856-859); Morrison ((1994) Nature 368, 812-13); Fishwild et al, ((1996) Nature Biotechnology 14, 845-51); Neuberger ((1996) Nature Biotechnology 14, 826); and Lonberg and Huszar ((1995) Intern. Rev. Immunol. 13 65-93).

5. Single Chain Antibodies and Fab Fragments

Single-chain antibodies specific to an antigenic protein of interest can also be used as a reagent in the invention (see e.g., U.S. Pat. No. 4,946,778). In addition, construction of Fab expression libraries (see e.g., Huse, et al., 1989 Science 246: 1275-1281) allow rapid and effective identification of monoclonal Fab fragments with the desired specificity for a protein, or derivatives, fragments, analogs or homologs thereof.

Any oligopeptide, polypeptide or protein that has been modified or derivatized may also serve as reagent in forming a reagent-bearing article of the invention. A common example of a derivatization is binding a label to an oligopeptide, polypeptide or protein. A label may be a luminescent label, or a reagent that is a member of a specific binding pair such as biotin, avidin, streptavidin, digoxin, digoxigenin, and the like. In addition an oligopeptide, polypeptide or protein may be chemically modified by any of a broad range of reagents such as those provided by Pierce Chemical Co., Rockford, Ill.

Organic Molecules, Antibiotics, Metabolites, and Drugs

Any of a broad range of synthetic organic molecules, antibiotics and their derivatives, metabolites, enzyme substrates and substrate analogs, enzyme inhibitors, chemical compounds that are members of a combinatorial library, other biochemical moieties, pharmaceutical candidates and lead compounds, and the like, all may serve as reagents of the present invention. By way of example, during a program to develop a new pharmaceutical agent for approval and marketing, a large number of candidate pharmaceutical agents are identified; certain of these survive winnowing experiments and are identified as lead compounds. As an additional example, many antibiotics are currently known, and many more are being identified. An antibiotic may be used as a reagent of the invention in various assays and processes. Metabolomics is a growing field of investigation as one of the consequences of genomics studies. A metabolite, or a suspected or candidate metabolite, may be bound to a particle to facilitate investigational and diagnostic research concerning the role played by the metabolite. In addition, any enzyme substrate or substrate analog, or an enzyme inhibitor, or a candidate inhibitor in a screen of an inhibitor library, may be a reagent of the invention. Eukaryotic proteins contain many post-translational modifications, of which complex glycosidic substituents are very important. Synthetic libraries of complex saccharides may be bound as the reagents in the particles of the invention. Components of combinatorial libraries in general may be bound as reagents to particles of the invention as part of investigational studies directed toward identifying and optimizing a chemical substance for use as a pharmaceutical agent.

Inorganic Molecules

Any inorganic compound can be a reagent of the invention. An important example is an inorganic substance that is a catalyst. Other examples of inorganic reagents of the invention include nanoparticles (e.g. quantum dots), ceramic particles, semiconductor particles, and the like.

Organelles, Viruses and Cells

A reagent bound to coded particle of the invention may be a supramolecular construct, a subcellular particle, or a complete cell. For example, liposomes and lipid vesicles may be bound as reagents. Such constructs may include within the lumen or bound within the lipid membrane a reagent or molecule of interest for a particular application. Any subcellular particle may be bound to a coded particle, including, by way of nonlimiting example, microsomes, ribonucleoprotein particles, ribosomes, particles of endoplasmic reticulum, particles of Golgi apparatus, lysosomes, proteasomes, peroxisomes, mitochondria, and so forth. Additionally whole cells may be bound to a coded particle, including, by way of nonlimiting example, fibroblasts, hepatocytes, myocytes, erythrocytes, kidney cells, lymphocytes, macrophages, adipocytes, pancreatic islet cells, glial cells, dendroctyes, bacterial cells including any of a wide range of pathogens, and virus particles.

Assay Compositions

The invention includes assay compositions that contain a reagent particle of the invention and a fluid medium. Commonly the reagent particle is suspended in the fluid. In many applications of assay compositions, they may in addition contain an analyte in the fluid. The fluid may be any gaseous or liquid fluid, or a it may be a supercritical fluid. Commonly a fluid may be an aqueous liquid, such as a buffer optimized to carry out a particular assay.

In addition, an assay composition may contain a reagent library that includes a plurality of reagent particles of the invention and a fluid medium. Such a composition may also have an analyte contained in the fluid.

Detection and Labeling

An analyte, a target molecule or a member of a specific binding pair bound the chemical moiety on an assay article may be detected in many ways. Detecting may include any one or more processes that result in the ability to observe the presence and or the amount of a bound target molecule. Physical, chemical or biological methods may be used to detect and quantitate a bound target molecule. Physical methods include, by way of nonlimiting example, surface plasmon resonance (SPR) detection, using SPR to detect binding of a bound target molecule to an immobilized probe, or having a probe in a chromatographic medium and detecting binding of a bound target molecule in the chromatographic medium. Physical methods further include a gel electrophoresis or capillary electrophoresis format in which bound target molecules are resolved from other molecules, and the resolved bound target molecules are detected. Chemical methods include hybridization methods and formation of specific binding pairs generally in which a bound target molecule binds to a probe. Biological methods include causing a bound target molecule to exert a biological effect on a cell, and detecting the effect. The present invention discloses examples of biological effects which may be used as a biological assay. In many embodiments, a bound target or member of a specific binding pair may be labeled as described below to assist in detection and quantitation.

For example, a sample nucleic acid may be labeled by chemical or enzymatic addition of a labeled moiety such as a labeled nucleotide or a labeled oligonucleotide linker. An alternative way of accomplishing detection is to use a labeled form of a bound target molecule, and to detect the bound label. A label may be a radioisotopic label, such as ¹²⁵I, ³⁵S, ³²P, ¹⁴C, or ³H, for example, that is detectable by its radioactivity. Alternatively, a label may be selected such that it can be detected using a spectroscopic method, for example. In one instance, a label may be a chromophore, absorbing incident ultraviolet, visible, infrared, microwave or similar electromagnetic radiation. A preferred label is one detectable by luminescence. Generally, luminescence refers the emission of electromagnetic radiation from a substance or a chemical. The radiation may occur in any region of the electromagnetic spectrum; i.e., the frequency of the emitted radiation may be anywhere in the spectrum. Commonly luminescence occurs in the ultraviolet, visible, or infrared spectral regions. Luminescence includes fluorescence, phosphorescence, and chemiluminescence. Thus a label that fluoresces, or that phosphoresces, or that induces a chemiluminscent reaction, may be employed. Nonlimiting examples of suitable fluorescent labels, or fluorochromes, include a ¹⁵² Eu label, a fluorescein label, a rhodamine label, a phycoerythrin label, a phycocyanin label, Cy-3, Cy-5, an allophycocyanin label, an o-phthalaldehyde label, and a fluorescamine label. Luminescent labels afford detection with high sensitivity. A label may furthermore be a magnetic resonance label, such as a stable free radical label detectable by electron paramagnetic resonance, or a nuclear label, detectable by nuclear magnetic resonance. A label may still further be a ligand in a specific ligand-receptor pair; the presence of the ligand is then detected by the secondary binding of an additional ligand-specific receptor, which commonly is itself labeled for detection. Nonlimiting examples of such ligand-receptor pairs include biotin and streptavidin or avidin, a hapten such as digoxigenin or antigen and its specific antibody, and so forth.

Detecting, quantitating, including labeling, methods are known generally to workers of skill in fields related to the present invention, including, by way of nonlimiting example, workers of skill in spectroscopy, nucleic acid chemistry, biochemistry, molecular biology and cell biology. Quantitating permits determining the quantity, mass, or concentration of a target molecule, or fragment thereof, that has bound to the probe. Quantitation includes determining the amount of change in a physical, chemical, or biological property as described in this and preceding paragraphs. For example the intensity of a signal originating from a label may be used to assess the quantity of the nucleic acid bound to the probe. Any equivalent process yielding a way of detecting the presence and/or the quantity, mass, or concentration of a polynucleotide or fragment thereof that detects a target molecule is envisioned to be within the scope of the present invention.

Methods of Assaying and Detecting a Process

The invention provides generally an assay article having a chemical attached or a set of assay articles to be used together, as well as methods employing these assay articles in a broad range of assays. By way of nonlimiting example, the following discussion addresses methods wherein the assay articles are beads or particles.

The particles include an optical substrate and are fabricated according to the methods described herein to have a diffraction grating disposed in the substrate, such that when illuminated with incident light, a signal emanating from the grating is characteristic of a particular code. The fabrication processes of the invention provide exceedingly high numbers of codes such that a degree of complexity in multiplexing experiments is possible. Thus, in principle and in practice (see the Examples) single beads can be employed and decoded independently of others. The availability of such a large number of coded particles permits attaching a correspondingly large number of chemicals to a set of particles, wherein each particle identifies the bound chemical according to its code. The bound chemical is the first specific binding substance of a specific binding pair, and targets the binding of a cognate specific binding substance that may be present as an analyte in a sample, to provide the specific binding pair bound to the particle. The presence and amount of the cognate specific binding substance bound to each coded particle is determined during the assay, such that the code identifies the first specific binding substance, and thereby identifies the cognate specific binding substance according to the specificity characterizing the specific binding pair. An extensive yet nonlimiting list of possible first specific binding substances includes a nucleic acid, a polynucleotide, an oligonucleotide, a nucleotide, a nucleoside, a protein nucleic acid, an oligopeptide nucleic acid, a protein or fragment thereof, an antibody or fragment thereof, an enzyme or fragment thereof, a receptor or fragment thereof, a polypeptide, an oligopeptide, an amino acid, a derivative of any of them, or a modification of any of them; a synthetic organic molecule, a synthetic intermediate, a synthetic precursor, an antibiotic, a metabolite, a candidate pharmaceutical agent, a pharmaceutical agent, a virus, a prokaryotic cell, a eukaryotic cell, a vertebrate cell, a mammalian cell, a human cell, a subcellular organelle, and a component of any them; and a polynucleotide that includes an allele of a single nucleotide polymorphism.

In order to conduct an assay, a particle or a set of particles is contacted with a sample believed to contain at least one analyte targeted by the chemicals on the particles, under conditions that promote binding of the analyte to the chemical. For example, for oligonucleotide probes, the conditions would promote specific hybridization of complementary nucleotide sequences under suitable conditions of stringency. After rinsing away excess sample solution, the presence of the target now bound to the particles, and the associated particle codes, is determined. In common practical implementations, the target(s) may themselves be labeled; in other common implementations the bound targets are further bound to specific detecting substances which may be labeled. For example, in hybridization assays for oligonucleotides, it is likely that the target oligonucleotide bears a label. In immunoassays, however, in a sandwich format, a secondary antibody that binds to a target antigen carries the label. For sensitivity and spectral specificity many labels are luminescent, i.e., they emit fluorescent, phosphorescent, or chemiluminescent radiation that is quantitated by a sensitive detector, such as, by way of nonlimiting example, a photomultiplier or CCD device. Numerous instances of assays are provided in the Examples below.

Use of Reagent-Bearing Articles in Assays

Referring to FIGS. 3-8, the substrate 10 of the optical identification element (or microbead) 8 may be functionalized by coating or attaching a desired probe 76, such as a compound, chemical or molecule, which is then used in an assay as an attractant for certain complimentary compounds, chemicals or molecules, otherwise known as a “target” analyte 52-54 (see FIG. 6). This capability to uniquely encode a large number of microbeads 8 with a corresponding unique probe 76 bound thereto enables these functionalized microbeads 72 to be mixed with unknown “target” analytes 52-54 to perform a multiplexed experiment. The procedure 40 for performing such a multiplexed assay or experiment includes the steps of producing (step 42) the microbead 8, as described hereinbefore, and functionalizing (step 44) the substrate 10 of the microbead 8 by coating/depositing/growing it with a probe 76 that will react in a predetermined way with “target” analytes 52-54. An assay is then performed (step 46) with a plurality of functionalized microbeads 72 with different identification codes 58 at the same time. In step 48, the fluorescence of the functionalized microbeads 72 is analyzed, and the functionalized microbead 72 is read to determine the code 58 thereof to thereby determine which “target” analytes 5-54 are present in the solution 60.

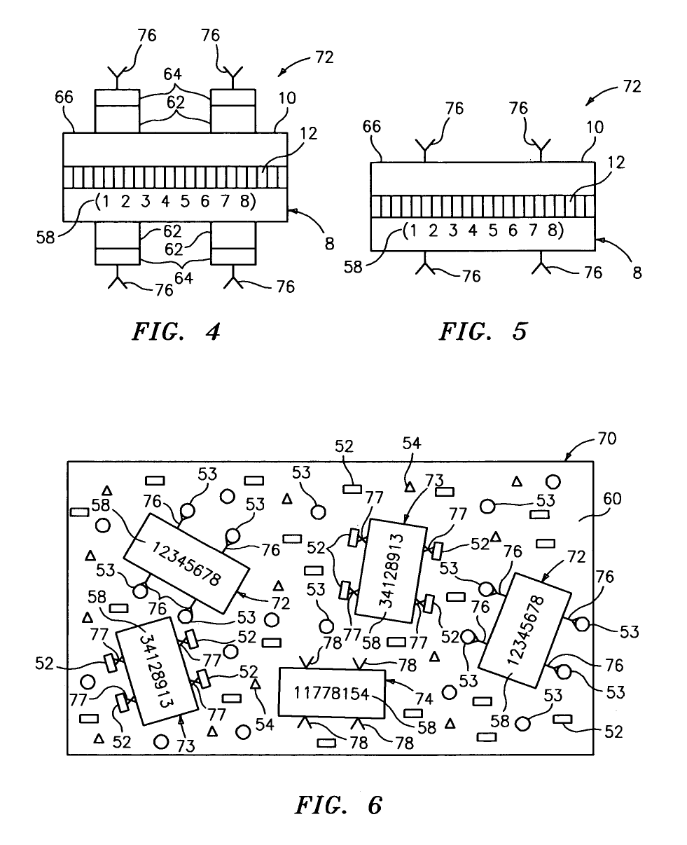

In FIGS. 4 and 5, a functionalized microbead 72 is shown, wherein the substrate 10 of the microbead 8 is coated with a probe 76 and used in an assay or as an attractant for certain “target” analytes 52-54 (see FIG. 6). In one embodiment shown in FIG. 4, the microbead 8 is coated with a linker molecule or complex 62 as is known in the art. A molecular group 64 is bound to the probe 76 to enable the probe to be bonded to the linker molecule or complex 62, and thus to the microbead 8 to form the functionalized microbead 72. The probe 76 may include one of an oligonucleotides (oligos), antibodies, peptides, amino acid strings, cDNA, RNA, chemicals, nucleic acid oligomers, polymers, biological cells, or proteins. For example, the probe 76 may comprise a single strand of DNA (or portion thereof) and the “target” analyte 52-54 comprises at least one unknown single strand of DNA, wherein each different “target” analyte has a different DNA sequence.

In some instances as shown in FIG. 5, the probe 76 may be bound directly to the substrate 10 of the microbead 8, or directly synthesized (or grown) thereon, such as via phosphoramidite chemistry. Examples of surface chemistry for the functionalized microbeads 72 include Streptavidin/biotinylated oligos and Aldehyde/amine modified oligos. Further, the microbead may be coated with a blocker of non-specific binding (e.g., salmon sperm DNA) to prevent bonding of analytes 52-54 (e.g. DNA) to the non-functionalized surface 66 of the functionalized microbeads 72.

Referring to FIG. 6, an assay is performed by adding a solution 60 of different types of “target” analytes 52-54 into a cell or container 70 having a plurality of functionalized microbeads 72-74 disposed therein. As discussed in step 46 of FIG. 3, the functionalized microbeads 72-74 placed in the cell 70 have different identification codes 58 that correspond to unique probes 76-78 bonded thereto. For example, all functionalized microbeads 72 disposed within the cell 70 having an identification code of 12345678 is coated with a unique probe 76. All functionalized microbeads 73 disposed within the cell 72 having an identification code of 34128913 is coated with a unique probe 77. All functionalized microbeads 77 disposed within the cell 70 having an identification code of 11778154 is coated with a unique probe 78.

The “target” analytes 52-54 within the solution 60 are then mixed with the functionalized microbeads 72-74. During the mixing of the “target” analytes 52-54 and the functionalized microbeads 72-74, the “target” analytes attach to the complementary probes 76-78, as shown for functionalized microbeads 72,73 having codes 12345678 and 34128913. Specifically, as shown in FIG. 6, “target” analytes 53 bonded with probes 76 of the functionalized microbeads 72 having the code 12345678, and “target” analytes 52 bonded with probes 77 of the functionalized microbeads 73 having the code 34128913. On the other hand, “target” analytes 54 did not bond with any probes, and not “target” analytes 52-54 in the solution 60 bonded with probes 78 of the functionalized microbeads 74 having the code 11778154. Consequently, knowing which “target” analytes attach to which probes along with the capability of identifying each probe by the encoded microbead, the results of the assay would show that the unknown “target” analytes in the solution 60 includes “target” analytes 53, 54, as will be described in further detail.

For example as discussed hereinbefore, each coded functionalized microbead 72-74 has a unique probe 76-78, respectively bonded thereto, such as a portion of a single strand of DNA. Similarly, the “target” analytes 52-54 comprise a plurality of unknown and unique single strands of DNA. These “target” analytes 52-54 are also processed with a fluorescent, such as dyeing, such that the test molecules illuminate. As will be discussed hereinafter, the fluorescence of the “target” analytes provide the means to identify, which functionalized microbeads 72-74 have a “target” analyte bound thereto.

Once the reaction or combining is complete, the functionalized microbeads 72-74 are rinsed off with a saline solution to clean off the uncombined “target” analytes 52-54. As shown in FIG. 7, the functionalized microbeads 72-74 may be placed in a tray 84 with grooves 82 to allow the functionalized microbeads to be aligned in a predetermined direction, such as that described in U.S. Patent Application Serial No. (Cidra Docket No. CC-0648), filed contemporaneously, which is incorporated herein by reference. The grooves 82 may have holes (not shown) that provide suction to keep the functionalized microbeads in position. Once aligned in the tray 84, the functionalized microbeads 52-54 are individually scanned and analyzed by the bead detector 20.

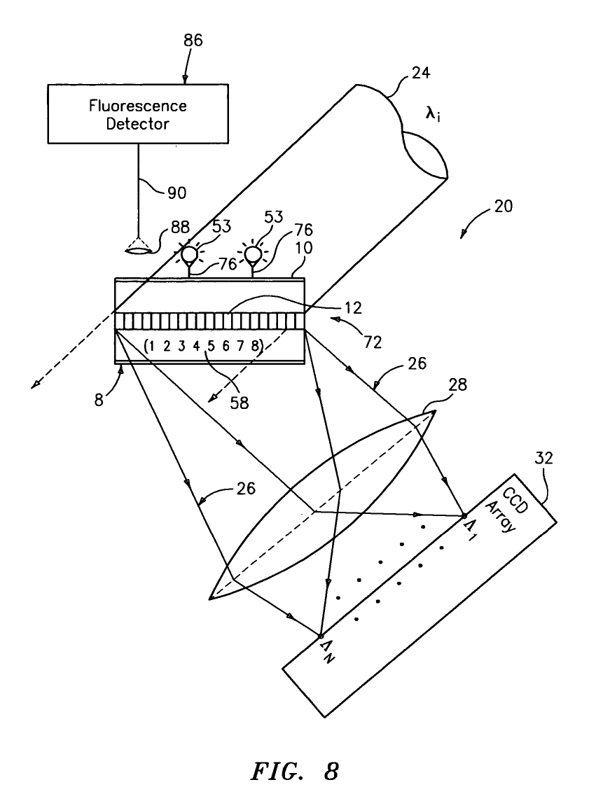

As best shown in FIG. 8, each functionalized microbead 72-74 is detected for fluorescence and analyzed to determine the identification code 58 of the functionalized microbeads. A light source (not shown) may be provided to luminate the microbeads 72-74. Once the fluorescent microbeads 72-74 are identified and knowing which probe 76-78 (or single strand of DNA) was bound to each coded, functionalized microbead 72-74, the bead detector 20 determines which “target” analytes 52-54 were present in the solution 60. As described hereinbefore, the bead detector 20 illuminates the functionalized microbeads 72-74 and focuses light 26 reflected by the diffraction grating 12 onto a CCD array or camera 32, whereby the code 58 of the functionalized microbead 72-74 is determined. Secondly, the bead detector 20 includes a fluorescence detector 86 for measuring the fluorescence emanating from “target” analytes 52-54 bound to the probes 76-78. The fluorescence meter 86 includes a lens 88 and optical fiber 90 for receiving and providing the fluorescence from the “target” analyte 52-54 to the fluorescence meter.

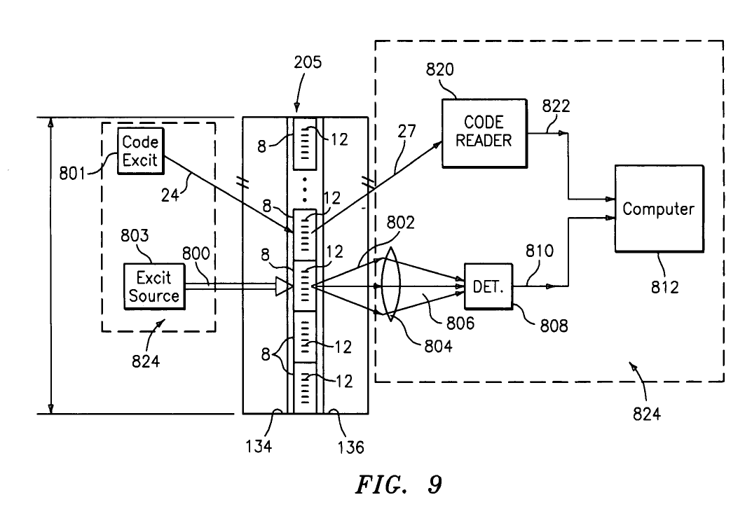

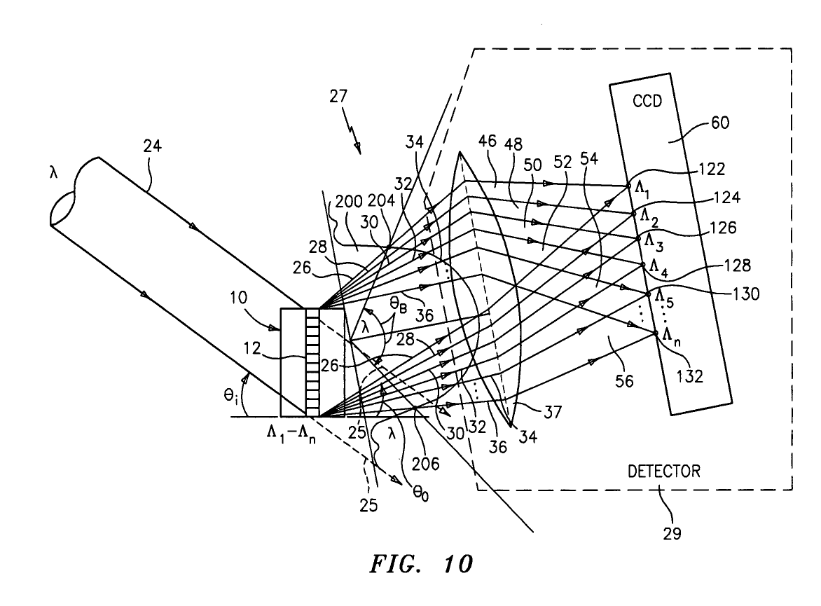

Referring to FIG. 9, more specifically, the codes in the microbeads 8 are detected when illuminated by incident light 24 which produces a diffracted or output light signal 27 to a reader 820, which includes the optics and electronics necessary to read the codes in each bead 8, as described herein and/or in the aforementioned copending patent application. The reader 820 provides a signal on a line 822 indicative of the code in each of the bead 8. The incident light 24 may be directed transversely from the side of the tray 84 (or from an end or any other angle) with a narrow band (single wavelength) and/or multiple wavelength source, in which case the code is represented by a spatial distribution of light and/or a wavelength spectrum, respectively, as described hereinafter and in the aforementioned copending patent application. Other illumination, readout techniques, types of gratings, geometries, materials, etc. may be used for the microbeads 8, as discussed hereinafter and in the aforementioned patent application.

For assays that use fluorescent molecule markers to label or tag chemicals, an optical excitation signal 800 is incident on the microbeads 8 through the tray 84 and a fluorescent optical output signal 802 emanates from the beads 8 that have the fluorescent molecule bound. The fluorescent optical output signal 802 passes through a lens 804, which provides focused light 802 to a known optical fluorescence detector 808. Instead of or in addition to the lens 802, other imaging optics may be used to provide the desired characteristics of the optical image/signal onto the fluorescence detector 808. The detector 808 provides an output signal on a line 810 indicative of the amount of fluorescence on a given bead 8, which can then be interpreted to determine what type of chemical is bound to the bead 10.

The tray 84 is made of glass or plastic or any material that is transparent to the code reading incident beam 24 and code reading output light beams 27 as well as the fluorescent excitation beam 800 and the output fluorescent optical signal 802, and is properly suited for the desired application or experiment, e.g., temperature range, harsh chemicals, or other application specific requirements.

The code signal 822 from the bead code reader 820 and the fluorescent signal 810 from the fluorescence detector are provided to a known computer 812. The computer reads the code associated with each bead and determines the chemical probe that was bound thereto from a predetermined table that correlates a predetermined relationship between the bead code and the bound probed. In addition, the computer 812 and reads the fluorescence associated with each bead and determines the sample or analyte that is bound to the bead from a predetermined table that correlates a predetermined relationship between the fluorescence tag and the analyte bound thereto. The computer 812 then determines information about the analyte and/or the probe as well as about the bonding of the analyte to the probe, and provides, such information on a display, printout, storage medium or other interface to an operator, scientist or database for review and/or analysis. The sources 801, 803 the code reader 820, the fluorescence optics 804 and detector 808 and the computer 812 may all be part of an assay reader 824.

Alternatively, instead of having the code excitation source 801 and the fluorescence excitation source 803, the reader 24 may have only one source beam which provides both the reflected optical signal 27 for determining the code and the fluorescence signal 802 for reading the tagged analyte bound to the beads 8. In that case the input optical signal is a common wavelength that performs both functions simultaneously, or sequentially, if desired.

Generally, the assay of the present invention may be used to carry out any binding assay or screen involving immobilization of one of the binding agents. Such solid-phase assays or screens are well known in the chemical and biochemical arts. For example, such screening may involve specific binding of cells to a molecule (e.g. an antibody or antigen) immobilized on a microbead followed by analysis to detect whether or to what extent binding occurs. Alternatively, the beads may subsequently be sorted and analyzed via flow cytometry (see e.g. by Needels et al. (1993). Examples of biological compounds that may be assayed or screened using the assay of the present invention include, e.g. agonists and antagonists for cell membrane receptors, toxins, venoms, viral epitopes, hormones, sugars, cofactors, peptides, enzyme substrates, drugs inclusive of opiates and steroids, proteins including antibodies, monoclonal antibodies, antisera reactive with specific antigenic determinants, nucleic acids, lectins, polysaccharides, cellular membranes and organelles. In addition, the present invention may be used in any of a large number of well-known hybridization assays where nucleic acids are immobilized on a surface of a substrate, e.g. genotyping, polymorphism detection, gene expression analysis, fingerprinting, and other methods of DNA- or RNA-based sample analysis or diagnosis.

Any of the great number of isotopic and non-isotopic labeling and detection methods well-known in the chemical and biochemical assay art may be used to detect binding with the present invention. Alternatively, spectroscopic methods well-known in the art may be used to determine directly whether a molecule is bound to a surface coating in a desired configuration. Spectroscopic methods include e.g., UV-VIS, NMR, EPR, IR, Raman, mass spectrometry and other methods well-known in the art. For example, mass spectrometry also is now widely employed for the analysis of biological macromolecules. The method typically involves immobilization of a protein on a surface of substrate where it is then exposed to a ligand binding interaction. Following ligand binding (or non-binding) the molecule is desorbed from the surface and into a spectrometer using a laser (see, e.g. Merchant and Weinberger, “Recent advancements in surface-enhanced laser desorption/ionization-time of flight-mass spectrometry,” Electrophoresis 21: 1164-1177 (2000)). The microbeads in the assay of the present invention may be used as substrates from which to input analytes in the mass spectrometry detection methods described above.

Various aspects of the present invention may be conducted in an automated or semi-automated manner, generally with the assistance of well-known data processing methods. Computer programs and other data processing methods well known in the art may be used to store information including e.g. microbead identifiers, probe sequence information, sample information, and binding signal intensities. Data processing methods well known in the art may be used to read input data covering the desired characteristics.

The invention may be used in many areas such as drug discovery, functionalized substrates, biology, proteomics, combinatorial chemistry, DNA analysis/tracking/sorting/tagging, as well as tagging of molecules, biological particles, matrix support materials, immunoassays, receptor binding assays, scintillation proximity assays, radioactive or non-radioactive proximity assays, and other assays, (including fluorescent, mass spectroscopy), high throughput drug/genome screening, and/or massively parallel assay applications. The invention provides uniquely identifiable beads with reaction supports by active coatings for reaction tracking to perform multiplexed experiments.

Some current techniques used in combinatorial chemistry or biochemistry are described in U.S. Pat. No. 6,294,327, entitled “Apparatus and Method for Detecting Samples Labeled With Material Having Strong Light Scattering Properties, Using Reflection Mode Light and Diffuse Scattering”, issued Sep. 23, 2001 to Walton et al.; U.S. Pat. No. 6,242,180, entitled “Computer Aided Visualization and Analysis System for Sequence Evaluation”, issued Jun. 5, 2001, to Chee; U.S. Pat. No. 6,309,823 entitled “Arrays of Nucleic Acid Probes for Analyzing Biotransformation of Genes and Methods of Using the Same”, Oct. 30, 2001, to Cronin et al.; U.S. Pat. No. 6,440,667, entitled “Analysis of Target Molecules Using an Encoding System”; U.S. Pat. No. 6,355,432, entitled “Products for Detecting Nucleic Acids”; U.S. Pat. No. 6,197,506, entitled “Method of Detecting Nucleic Acids”; U.S. Pat. No. 6,309,822, entitled “Method for comparing copy number of nucleic acid sequences”; U.S. Pat. No. 5,547,839, entitled “Sequencing of surface immobilized polymers utilizing micro-fluorescence detection”, U.S. Pat. No. 6,383,754, entitled “Binary Encoded Sequence Tags”, and U.S. Pat. No. 6,383,754, entitled “Fixed Address Analysis of Sequence Tags”, which are all incorporated herein by reference to the extent needed to understand the present invention.

The invention can be used in combinatorial chemistry, active coating and functionalized polymers, as well as immunoassays, and hybridization reactions. The invention enables millions of parallel chemical reactions, enable large-scale repeated chemical reactions, increase productivity and reduce time-to-market for drug and other material development industries.

As discussed hereinbefore, although a fluorescent label is probably most convenient, other sorts of labels, e.g., radioactive, enzyme linked, optically detectable, or spectroscopic labels may be used. An appropriate detection method applicable to the selected labeling method can be selected. Suitable labels include radionucleotides, enzymes, substrates, cofactors, inhibitors magnetic particles, heavy metal atoms, and particularly fluorescers, chemiluminescers, and spectroscopic labels. Patents teaching the use of such labels include U.S. Pat. Nos. 3,817,837; 3,850,752; 3,939,350; 3,996,345; 4,277,437; 4,275,149; and 4,366,241.

With an appropriate label selected, the detection system best adapted for high resolution and high sensitivity detection may be selected. As indicated above, an optically detectable system, e.g., fluorescence or chemilumnescence would be preferred but is not required. Other detection systems may be adapted to the purpose, e.g., electron microscopy, scanning electron microscopy (SEM), scanning tunneling electron microscopy (STEM), infrared microscopy, atomic force microscopy (AFM), electrical conductance, and image plate transfer.

Optical Particle

As described above, a significant embodiment of an optical particle of the invention is represented as a diffraction grating-based encoded element (or encoded element or coded element) that includes an optical substrate. The substrate has an optical diffraction grating disposed (or written, impressed, embedded, imprinted, etched, grown, deposited or otherwise formed) in the volume of or on a surface thereof. The grating is a periodic or aperiodic variation in the effective refractive index and/or effective optical absorption of at least a portion of the substrate.

Referring to FIG. 10, the reflected light 27, comprises a plurality of beams 26-36 that pass through a lens 37, which provides focused light beams 46-56, respectively, which are imaged onto a CCD camera 60. The lens 37 and the camera 60, and any other necessary electronics or optics for performing the functions described herein, make up the reader 29. Instead of or in addition to the lens 37, other imaging optics may be used to provide the desired characteristics of the optical image/signal onto the camera 60 (e.g., spots, lines, circles, ovals, etc.), depending on the shape of the substrate 10 and input optical signals. Also, instead of a CCD camera other devices may be used to read/capture the output light.

Referring to FIG. 11, the image on the CCD camera 60 is a series of illuminated stripes indicating ones and zeros of a digital pattern or code of the grating 12 in the element 8. Referring to FIG. 12, lines 68 on a graph 70 are indicative of a digitized version of the image of FIG. 11 as indicated in spatial periods (Λ1-Λn).

Each of the individual spatial periods (Λ1-Λn) in the grating 12 is slightly different, thus producing an array of N unique diffraction conditions (or diffraction angles) discussed more hereinafter. When the element 8 is illuminated from the side, in the region of the grating 12, at an appropriate input angle, e.g., about 30 degrees, with a single input wavelength λ (monochromatic) source, the diffracted (or reflected) beams 26-36 are generated. Other input angles θi may be used if desired, depending on various design parameters as discussed herein and/or in the aforementioned patent application, and provided that a known diffraction equation (Eq. 1 below) is satisfied:
sin(θᵢ)+sin(θₒ)=mλ/nΛ Eq. 1
where Eq. 1 is diffraction (or reflection or scatter) relationship between input wavelength λ, input incident angle θi, output incident angle θo, and the spatial period Λ of the grating 12. Further, m is the “order” of the reflection being observed, and n is the refractive index of the substrate 10. The value of m=1 or first order reflection is acceptable for illustrative purposes. Eq. 1 applies to light incident on outer surfaces of the substrate 10 which are parallel to the longitudinal axis of the grating (or the k_{B} vector). Because the angles θi,θo are defined outside the substrate 10 and because the effective refractive index of the substrate 10 is substantially a common value, the value of n in Eq. 1 cancels out of this equation.

Thus, for a given input wavelength λ, grating spacing Λ, and incident angle of the input light θi, the angle θo of the reflected output light may be determined. Solving Eq. 1 for θo and plugging in m=1, gives:
θo=sin⁻¹(λ/Λ−sin(θi)) Eq. 2

For example, for an input wavelength λ=532 nm, a grating spacing Λ=0.532 microns (or 532 nm), and an input angle of incidence θi=30 degrees, the output angle of reflection will be θo=30 degrees. Alternatively, for an input wavelength λ=632 nm, a grating spacing Λ=0.532 microns (or 532 nm), and an input angle θi of 30 degrees, the output angle of reflection θo will be at 43.47 degrees, or for an input angle θi=37 degrees, the output angle of reflection will be θo=37 degrees. Any input angle that satisfies the design requirements discussed herein and/or in the aforementioned patent application may be used.

In addition, to have sufficient optical output power and signal to noise ratio, the output light 27 should fall within an acceptable portion of the Bragg envelope (or normalized reflection efficiency envelope) curve 200, as indicated by points 204,206, also defined as a Bragg envelope angle θB, as also discussed herein and/or in the aforementioned patent application. The curve 200 may be defined as:

$$I ( ki , ko ) \approx {[ KD ]}^{2} sin {c}^{2} [ \frac{( ki - ko ) D}{2} ] \qquad Eq . 3$$

where K=2πδn/λ, where, δn is the local refractive index modulation amplitude of the grating and λ is the input wavelength, sinc(x)=sin(x)/x, and the vectors kᵢ=2πcos(θᵢ)/λ and kₒ=2πcos (θₒ)/λ are the projections of the incident light and the output (or reflected) light, respectively, onto the line 203 normal to the axial direction of the grating 12 (or the grating vector k_{B}), D is the thickness or depth of the grating 12 as measured along the line 203 (normal to the axial direction of the grating 12). Other substrate shapes than a cylinder may be used and will exhibit a similar peaked characteristic of the Bragg envelope. We have found that a value for δn of about 10⁻⁴ in the grating region of the substrate is acceptable; however, other values may be used if desired.

Rewriting Eq. 3 gives the reflection efficiency profile of the Bragg envelope as:

$$I ( ki , ko ) \approx {{[ \frac{2 π \cdot δ n \cdot D}{λ} ]}^{2} [ \frac{Sin ( x )}{x} ]}^{2} \qquad Eq . 4$$

where:
x=(ki−ko)D/2=(πD/λ)*(cos θ i−cos θo)

Thus, when the input angle θi is equal to the output (or reflected) angle θₒ (i.e., θi=θₒ), the reflection efficiency I (Eqs. 3 & 4) is maximized, which is at the center or peak of the Bragg envelope. When θi=θo, the input light angle is referred to as the Bragg angle as is known. The efficiency decreases for other input and output angles (i.e., θi ≠θₒ), as defined by Eqs. 3 & 4. Thus, for maximum reflection efficiency and thus output light power, for a given grating pitch Λ and input wavelength, the angle θi of the input light 24 should be set so that the angle θo of the reflected output light equals the input angle θi.

Also, as the thickness or diameter D of the grating decreases, the width of the sin(x)/x function (and thus the width of the Bragg envelope) increases and, the coefficient to or amplitude of the sinc²(or (sin(x)/x)² function (and thus the efficiency level across the Bragg envelope) also increases, and vice versa. Further, as the wavelength λ increases, the half-width of the Bragg envelope as well as the efficiency level across the Bragg envelope both decrease. Thus, there is a trade-off between the brightness of an individual bit and the number of bits available under the Bragg envelope. Ideally, δn should be made as large as possible to maximize the brightness, which allows D to be made smaller.

From Eq. 3 and 4, the half-angle of the Bragg envelope θ_{B} is defined as:

$${θ}_{B} = \frac{η λ}{π D sin ( {θ}_{i} )} \qquad Eq . 5$$

where η is a reflection efficiency factor which is the value for x in the sinc²(x) function where the value of sinc²(x) has decreased to a predetermined value from the maximum amplitude as indicated by points 204,206 on the curve 200.

We have found that the reflection efficiency is acceptable when η≦1.39. This value for η corresponds to when the amplitude of the reflected beam (i.e., from the sinc²(x) function of Eqs. 3 & 4) has decayed to about 50% of its peak value. In particular, when x=1.39=η, sinc²(x)=0.5. However, other values for efficiency thresholds or factor in the Bragg envelope may be used if desired.

The beams 26-36 are imaged onto the CCD camera 60 to produce the pattern of light and dark regions 120-132 representing a digital (or binary) code, where light=1 and dark=0 (or vice versa). The digital code may be generated by selectively creating individual index variations (or individual gratings) with the desired spatial periods Λ1-Λn. Other illumination, readout techniques, types of gratings, geometries, materials, etc. may be used as discussed in the aforementioned patent application.

Referring to FIG. 13, illustrations (a)-(c), for the grating 12 in a cylindrical substrate 10 having a sample spectral 17 bit code (i.e., 17 different pitches Λ1-Λ17), the corresponding image on the CCD (Charge Coupled Device) camera 60 is shown for a digital pattern of 7 bits turned on (10110010001001001); 9 bits turned on of (11000101010100111); all 17 bits turned on of (11111111111111111).

For the images in FIG. 13, the length of the substrate 10 was 450 microns, the outer diameter D1 was 65 microns, the inner diameter D was 14 microns, δn for the grating 12 was about 10⁻⁴, n1 in portion 20 was about 1.458 (at a wavelength of about 1550 nm), n2 in portion 18 was about 1.453, the average pitch spacing Λ for the grating 12 was about 0.542 microns, and the spacing between pitches ΔΛ was about 0.36% of the adjacent pitches A.

Referring to FIG. 14, illustration (a), the pitch Λ of an individual grating is the axial spatial period of the sinusoidal variation in the refractive index n1 in the region 20 of the substrate 10 along the axial length of the grating 12 as indicated by a curve 90 on a graph 91. Referring to FIG. 14, illustration (b), a sample composite grating 12 comprises three individual gratings that are co-located on the substrate 10, each individual grating having slightly different pitches, Λ1, Λ2, Λ3, respectively, and the difference (or spacing) ΔΛ between each pitch Λ being about 3.0% of the period of an adjacent pitch Λ as indicated by a series of curves 92 on a graph 94. Referring to FIG. 14, illustration (c), three individual gratings, each having slightly different pitches, Λ1, Λ2, Λ3, respectively, are shown, the difference ΔΛ between each pitch Λ being about 0.3% of the pitch Λ of the adjacent pitch as shown by a series of curves 95 on a graph 97. The individual gratings in FIG. 14, illustrations (b) and (c) are shown to all start at 0 for illustration purposes; however, it should be understood that, the separate gratings need not all start in phase with each other. Referring to FIG. 14, illustration (d), the overlapping of the individual sinusoidal refractive index variation pitches Λ1-Λn in the grating region 20 of the substrate 10, produces a combined resultant refractive index variation in the composite grating 12 shown as a curve 96 on a graph 98 representing the combination of the three pitches shown in FIG. 14, illustration (b). Accordingly, the resultant refractive index variation in the grating region 20 of the substrate 10 may not be sinusoidal and is a combination of the individual pitches Λ (or index variation).

The maximum number of resolvable bits N, which is equal to the number of different grating pitches Λ (and hence the number of codes), that can be accurately read (or resolved) using side-illumination and side-reading of the grating 12 in the substrate 10, is determined by numerous factors, including: the beam width w incident on the substrate (and the corresponding substrate length L and grating length Lg), the thickness or diameter D of the grating 12, the wavelength λ of incident light, the beam divergence angle θ_{R}, and the width of the Bragg envelope θ_{B} (discussed more in the aforementioned patent application), and may be determined by the equation:

$$N ≅ \frac{η β L}{2 D sin ( {θ}_{i} )} \qquad Eq . 6$$

Referring to FIG. 15, instead of having the input light 24 at a single wavelength λ (monochromatic) and reading the bits by the angle θo of the output light, the bits (or grating pitches A) may be read/detected by providing a plurality of wavelengths and reading the wavelength spectrum of the reflected output light signal. In this case, there would be one bit per wavelength, and thus, the code is contained in the wavelength information of the reflected output signal.

In this case, each bit (or Λ) is defined by whether its corresponding wavelength falls within the Bragg envelope, not by its angular position within the Bragg envelope 200. As a result, it is not limited by the number of angles that can fit in the Bragg envelope 200 for a given composite grating 12, as in the embodiment discussed hereinbefore. Thus, using multiple wavelengths, the only limitation in the number of bits N is the maximum number of grating pitches Λ that can be superimposed and optically distinguished in wavelength space for the output beam.

Referring to FIGS. 15 and 16, illustration (a), the reflection wavelength spectrum (λ1-λn) of the reflected output beam 310 will exhibit a series of reflection peaks 695, each appearing at the same output Bragg angle θo. Each wavelength peak 695 (λ1-λn) corresponds to an associated spatial period (Λ1-Λn), which make up the grating 12.

One way to measure the bits in wavelength space is to have the input light angle θi equal to the output light angle θo, which is kept at a constant value, and to provide an input wavelength λ that satisfies the diffraction condition (Eq. 1) for each grating pitch A. This will maximize the optical power of the output signal for each pitch Λ detected in the grating 12.

Referring to 16, illustration (b), the transmission wavelength spectrum of the transmitted output beam 330 (which is transmitted straight through the grating 12) will exhibit a series of notches (or dark spots) 696. Alternatively, instead of detecting the reflected output light 310, the transmitted light 330 may be detected at the detector/reader 308. It should be understood that the optical signal levels for the reflection peaks 695 and transmission notches 696 will depend on the “strength” of the grating 12, i.e., the magnitude of the index variation n in the grating 12.

In FIG. 15, the bits may be detected by continuously scanning the input wavelength. A known optical source 300 provides the input light signal 24 of a coherent scanned wavelength input light shown as a graph 304. The source 300 provides a sync signal on a line 306 to a known reader 308. The sync signal may be a timed pulse or a voltage ramped signal, which is indicative of the wavelength being provided as the input light 24 to the substrate 10 at any given time. The reader 308 may be a photodiode, CCD camera, or other optical detection device that detects when an optical signal is present and provides an output signal on a line 309 indicative of the code in the substrate 10 or of the wavelengths present in the output light, which is directly related to the code, as discussed herein. The grating 12 reflects the input light 24 and provides an output light signal 310 to the reader 308. The wavelength of the input signal is set such that the reflected output light 310 will be substantially in the center 314 of the Bragg envelope 200 for the individual grating pitch (or bit) being read.

Alternatively, the source 300 may provide a continuous broadband wavelength input signal such as that shown as a graph 316. In that case, the reflected output beam 310 signal is provided to a narrow band scanning filter 318 which scans across the desired range of wavelengths and provides a filtered output optical signal 320 to the reader 308. The filter 318 provides a sync signal on a line 322 to the reader, which is indicative of which wavelengths are being provided on the output signal 320 to the reader and may be similar to the sync signal discussed hereinbefore on the line 306 from the source 300. In this case, the source 300 does not need to provide a sync signal because the input optical signal 24 is continuous. Alternatively, instead of having the scanning filter being located in the path of the output beam 310, the scanning filter may be located in the path of the input beam 24 as indicated by the dashed box 324, which provides the sync signal on a line 323.

Alternatively, instead of the scanning filters 318,324, the reader 308 may be a known optical spectrometer (such as a known spectrum analyzer), capable of measuring the wavelength of the output light.

The desired values for the input wavelengths λ (or wavelength range) for the input signal 24 from the source 300 may be determined from the Bragg condition of Eq. 1, for a given grating spacing Λ and equal angles for the input light θi and the angle light θo. Solving Eq. 1 for λ and plugging in m=1, gives:
λ=Λ[sin(θo)+sin(θi)] Eq. 7

It is also possible to combine the angular-based code detection with the wavelength-based code detection, both discussed hereinbefore. In this case, each readout wavelength is associated with a predetermined number of bits within the Bragg envelope. Bits (or grating pitches Λ) written for different wavelengths do not show up unless the correct wavelength is used.

Accordingly, the bits (or grating pitches Λ) can be read using one wavelength and many angles, many wavelengths and one angle, or many wavelengths and many angles.

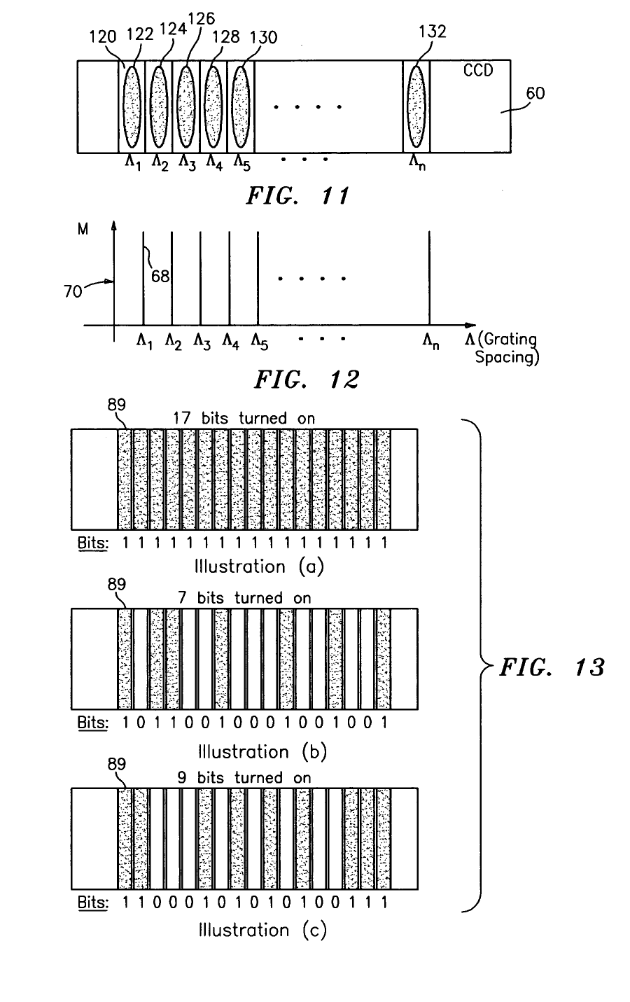

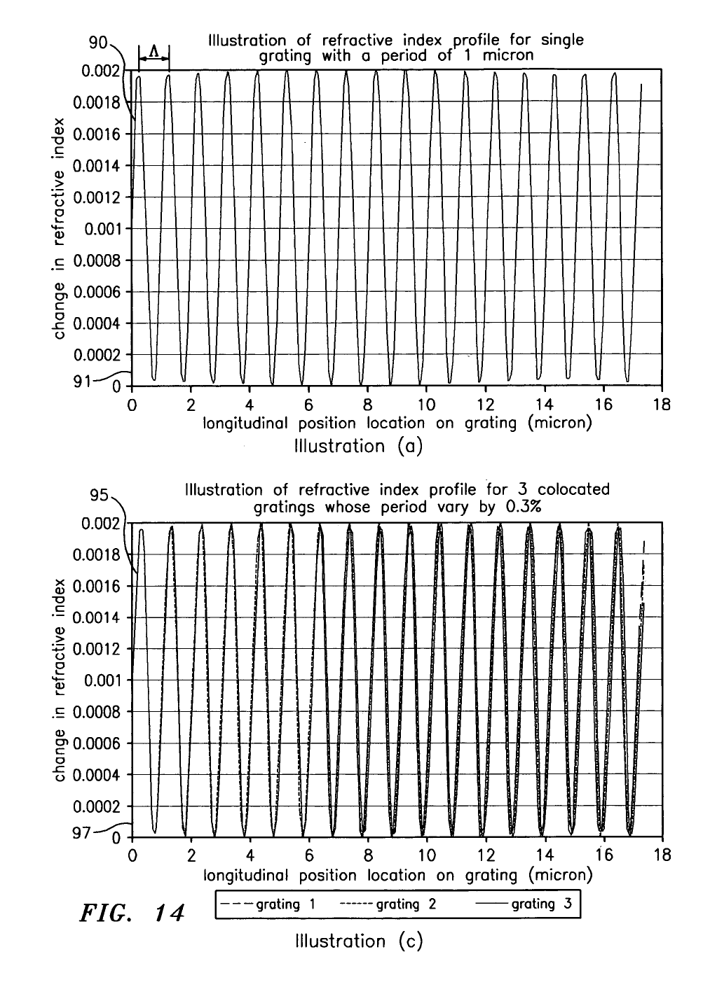

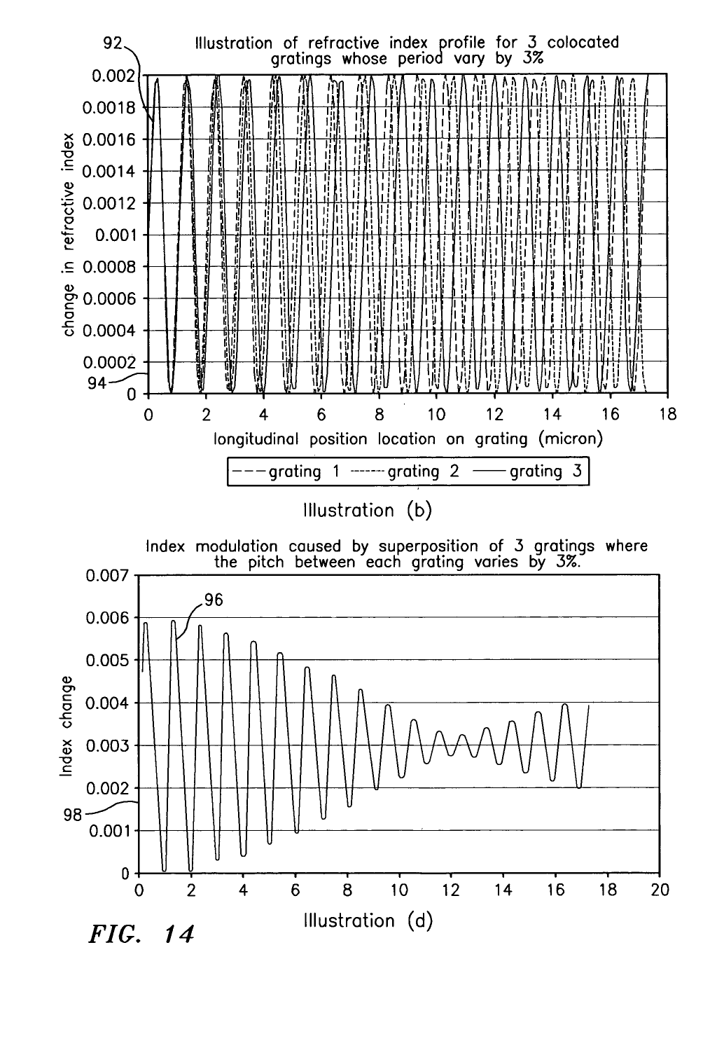

Referring to FIG. 17, the grating 12 may have a thickness or depth D which is comparable or smaller than the incident beam wavelength λ. This is known as a “thin” diffraction grating (or the full angle Bragg envelope is 180 degrees). In that case, the half-angle Bragg envelope θB is substantially 90 degrees; however, δn must be made large enough to provide sufficient reflection efficiency, per Eqs. 3 and 4. In particular, for a “thin” grating, D*δn≈λ/2, which corresponds to a π phase shift between adjacent minimum and maximum refractive index values of the grating 12.

It should be understood that there is still a trade-off discussed hereinbefore with beam divergence angle θ_{R} and the incident beam width (or length L of the substrate), but the accessible angular space is theoretically now 90 degrees. Also, for maximum efficiency, the phase shift between adjacent minimum and maximum refractive index values of the grating 12 should approach a π phase shift; however, other phase shifts may be used.

In this case, rather than having the input light 24 coming in at the conventional Bragg input angle θi, as discussed hereinbefore and indicated by a dashed line 701, the grating 12 is illuminated with the input light 24 oriented on a line 705 orthogonal to the longitudinal grating vector 705. The input beam 24 will split into two (or more) beams of equal amplitude, where the exit angle θₒ can be determined from Eq. 1 with the input angle θᵢ=0 (normal to the longitudinal axis of the grating 12).

In particular, from Eq. 1, for a given grating pitch Λ1, the +/−1^{st} order beams (m=+1 and m=−1), corresponds to output beams 700,702, respectively. For the +/−2^{nd} order beams (m=+2 and m=−2), corresponds to output beams 704,706, respectively. The 0^{th} order (undefracted) beam (m=0), corresponds to beam 708 and passes straight through the substrate. The output beams 700-708 project spectral spots or peaks 710-718, respectively, along a common plane, shown from the side by a line 709, which is parallel to the upper surface of the substrate 10.

For example, for a grating pitch Λ=1.0 um, and an input wavelength λ=400 nm, the exit angles θₒ are ˜+/−23.6 degrees (for m=+/−1), and +/−53.1 degrees (from m=+/−2), from Eq. 1. It should be understood that for certain wavelengths, certain orders (e.g., m=+/−2) may be reflected back toward the input side or otherwise not detectable at the output side of the grating 12.

Alternatively, one can use only the +/−1^{st} order (m=+/−1) output beams for the code, in which case there would be only 2 peaks to detect, 712, 714. Alternatively, one can also use any one or more pairs from any order output beam that is capable of being detected. Alternatively, instead of using a pair of output peaks for a given order, an individual peak may be used.

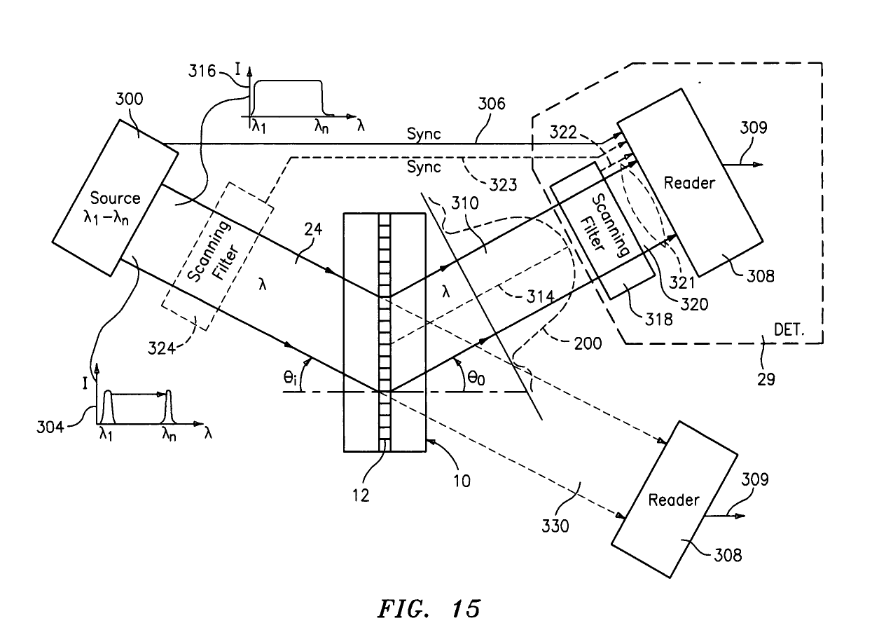

Referring to FIG. 18, if two pitches Λ1,Λ2 exist in the grating 12, two sets of peaks will exist. In particular, for a second grating pitch Λ2, the +/−1^{st} order beams (m=+1 and m=−1), corresponds to output beams 720,722, respectively. For the +/−2^{nd} order beams (m=+2 and m=−2), corresponds to output beams 724,726, respectively. The 0^{th} order (un-defracted) beam (m=0), corresponds to beam 718 and passes straight through the substrate. The output beams 720-726 corresponding to the second pitch Λ2 project spectral spots or peaks 730-736, respectively, which are at a different location than the point 710-716, but along the same common plane, shown from the side by the line 709.

Thus, for a given pitch Λ (or bit) in a grating, a set of spectral peaks will appear at a specific location in space. Thus, each different pitch corresponds to a different elevation or output angle which corresponds to a predetermined set of spectral peaks. Accordingly, the presence or absence of a particular peak or set of spectral peaks defines the code.

In general, if the angle of the grating 12 is not properly aligned with respect to the mechanical longitudinal axis of the substrate 10, the readout angles may no longer be symmetric, leading to possible difficulties in readout. With a thin grating, the angular sensitivity to the alignment of the longitudinal axis of the substrate 10 to the input angle θi of incident radiation is reduced or eliminated. In particular, the input light can be oriented along substantially any angle θi with respect to the grating 12 without causing output signal degradation, due the large Bragg angle envelope. Also, if the incident beam 24 is normal to the substrate 10, the grating 12 can be oriented at any rotational (or azimuthal) angle without causing output signal degradation. However, in each of these cases, changing the incident angle θi will affect the output angle θo of the reflected light in a predetermined predictable way, thereby allowing for accurate output code signal detection or compensation.

Referring to FIG. 19, for a thin grating, in addition to multiplexing in the elevation or output angle based on grating pitch A, the bits can also be multiplexed in an azimuthal (or rotational) angle θa of the substrate. In particular, a plurality of gratings 750,752,754,756 each having the same pitch Λ are disposed in a surface 701 of the substrate 10 and located in the plane of the substrate surface 701. The input light 24 is incident on all the gratings 750,752,754,756 simultaneously. Each of the gratings provides output beams oriented based on the grating orientation. For example, the grating 750 provides the output beams 764,762, the grating 752 provides the output beams 766,768, the grating 754 provides the output beams 770,772, and the grating 756 provides the output beams 774,776. Each of the output beams provides spectral peaks or spots (similar to that discussed hereinbefore), which are located in a plane 760 that is parallel to the substrate surface plane 701. In this case, a single grating pitch Λ can produce many bits depending on the number of gratings that can be placed at different azimuthal (rotational) angles on the surface of the substrate 10 and the number of output beam spectral peaks that can be spatially and optically resolved/detected. Each bit may be viewed as the presence or absence of a pair of peaks located at a predetermined location in space in the plane 760. Note that this example uses only the m=+/−1^{st} order for each reflected output beam. Alternatively, the detection may also use the m=+/−2^{nd} order. In that case, there would be two additional output beams and peaks (not shown) for each grating (as discussed hereinbefore) that may lie in the same plane as the plane 760 and may be on a concentric circle outside the circle 760.

In addition, the azimuthal multiplexing can be combined with the elevation or output angle multiplexing discussed hereinbefore to provide two levels of multiplexing. Accordingly, for a thin grating, the number of bits can be multiplexed based on the number of grating pitches Λ and/or geometrically by the orientation of the grating pitches.

Furthermore, if the input light angle θi is normal to the substrate 10, the edges of the substrate 10 no longer scatter light from the incident angle into the “code angular space”, as discussed herein and/or in the aforementioned patent application.

Also, in the thin grating geometry, a continuous broadband wavelength source may be used as the optical source if desired.

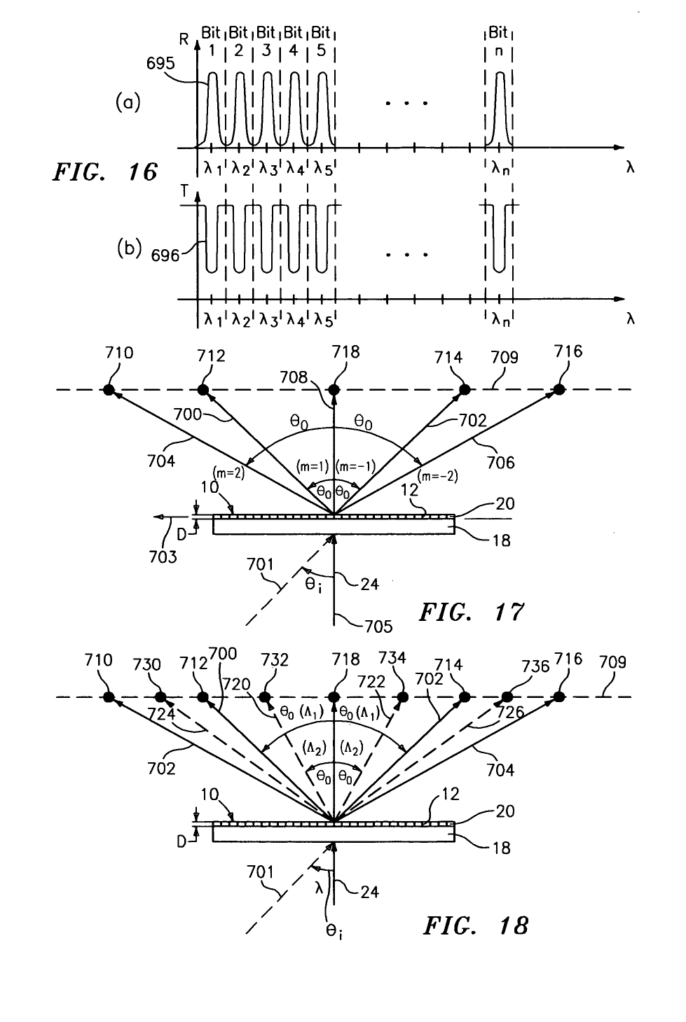

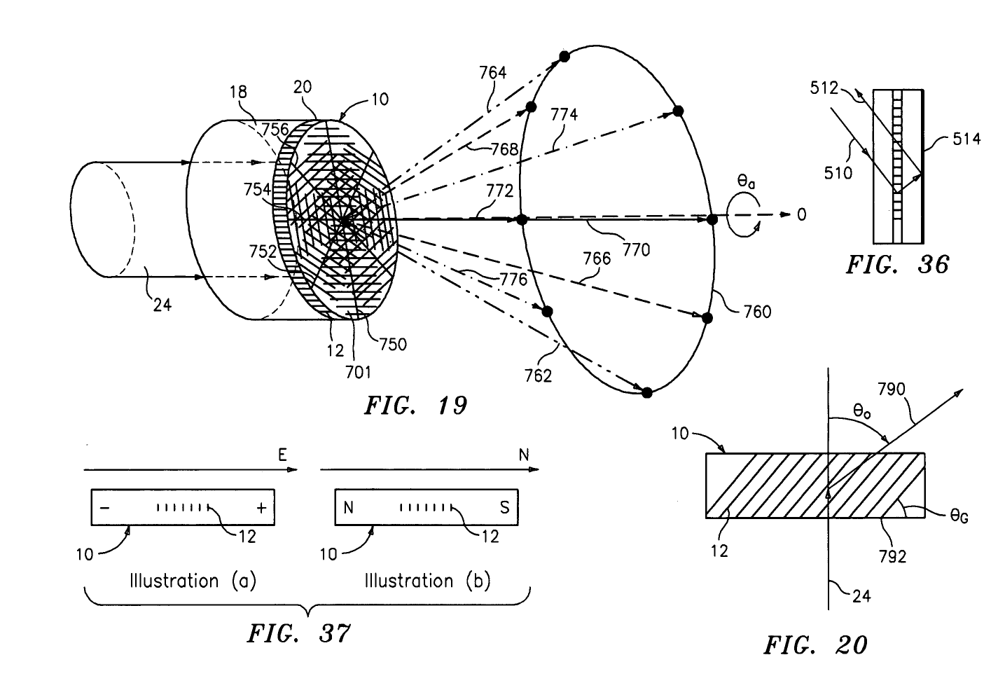

Referring to FIG. 20, instead of or in addition to the pitches Λ in the grating 12 being oriented normal to the longitudinal axis, the pitches may be created at a angle θg. In that case, when the input light 24 is incident normal to the surface 792, will produce a reflected output beam 790 having an angle θo determined by Eq. 1 as adjusted for the blaze angle θg. This can provide another level of multiplexing bits in the code.

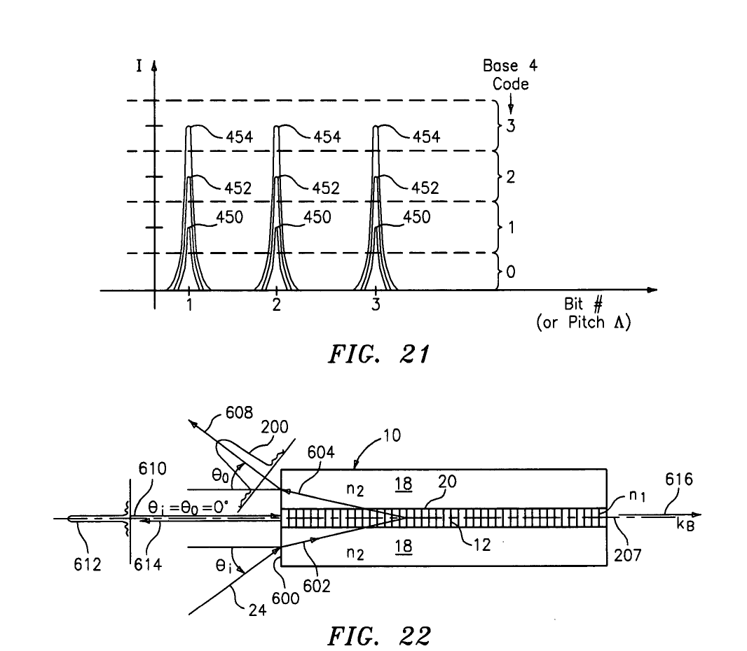

Referring to FIG. 21, instead of using an optical binary (0-1) code, an additional level of multiplexing may be provided by having the optical code use other numerical bases, if intensity levels of each bit are used to indicate code information. This could be achieved by having a corresponding magnitude (or strength) of the refractive index change (δn) for each grating pitch Λ. Four intensity ranges are shown for each bit number or pitch Λ, providing for a Base-4 code (where each bit corresponds to 0, 1, 2, or 3). The lowest intensity level, corresponding to a 0, would exist when this pitch Λ is not present in the grating 12. The next intensity level 450 would occur when a first low level δn1 exists in the grating that provides an output signal within the intensity range corresponding to a 1. The next intensity level 452 would occur when a second higher level δn2 exists in the grating 12 that provides an output signal within the intensity range corresponding to a 2. The next intensity level 452, would occur when a third higher level δn3 exists in the grating 12 that provides an output signal within the intensity range corresponding to a 3.

Referring to FIG. 22, the input light 24 may be incident on the substrate 10 on an end face 600 of the substrate 10. In that case, the input light 24 will be incident on the grating 12 having a more significant component of the light (as compared to side illumination discussed hereinbefore) along the longitudinal grating axis 207 of the grating (along the grating vector k_{B}), as shown by a line 602. The light 602 reflects off the grating 12 as indicated by a line 604 and exits the substrate as output light 608. Accordingly, it should be understood by one skilled in the art that the diffraction equations discussed hereinbefore regarding output diffraction angle θo also apply in this case except that the reference axis would now be the grating axis 207. Thus, in this case, the input and output light angles θi,θo, would be measured from the grating axis 207 and length Lg of the grating 12 would become the thickness or depth D of the grating 12. As a result, a grating 12 that is 400 microns long, would result in the Bragg envelope 200 being narrow. It should be understood that because the values of n1 and n2 are close to the same value, the slight angle changes of the light between the regions 18,20 are not shown herein.

In the case where incident light 610 is incident along the same direction as the grating vector (Kb) 207, i.e., θi=0 degrees, the incident light sees the whole length Lg of the grating 12 and the grating provides a reflected output light angle θo=0 degrees, and the Bragg envelope 612 becomes extremely narrow, as the narrowing effect discussed above reaches a limit. In that case, the relationship between a given pitch Λ in the grating 12 and the wavelength of reflection λ is governed by a known “Bragg grating” relation:
λ=2n_{eff}Λ Eq. 8
where n_{eff} is the effective index of refraction of the substrate, λ is the input (and output wavelength) and Λ is the pitch. This relation, as is known, may be derived from Eq. 1 where θi=θo=90 degrees.

In that case, the code information is readable only in the spectral wavelength of the reflected beam, similar to that discussed hereinbefore for wavelength based code reading. Accordingly, the input signal in this case may be a scanned wavelength source or a broadband wavelength source. In addition, as discussed hereinbefore for wavelength based code reading, the code information may be obtained in reflection from the reflected beam 614 or in transmission by the transmitted beam 616 that passes through the grating 12.

It should be understood that for shapes of the substrate 10 or element 8 other than a cylinder, the effect of various different shapes on the propagation of input light through the element 8, substrate 10, and/or grating 12, and the associated reflection angles, can be determined using known optical physics including Snell's Law, shown below:
nᵢₙ sin θin =nₒᵤₜ sin θout Eq. 9
where nᵢₙ is the refractive index of the first (input) medium, and nₒᵤₜ is the refractive index of the second (output) medium, and θin and θout are measured from a line 620 normal to an incident surface 622.

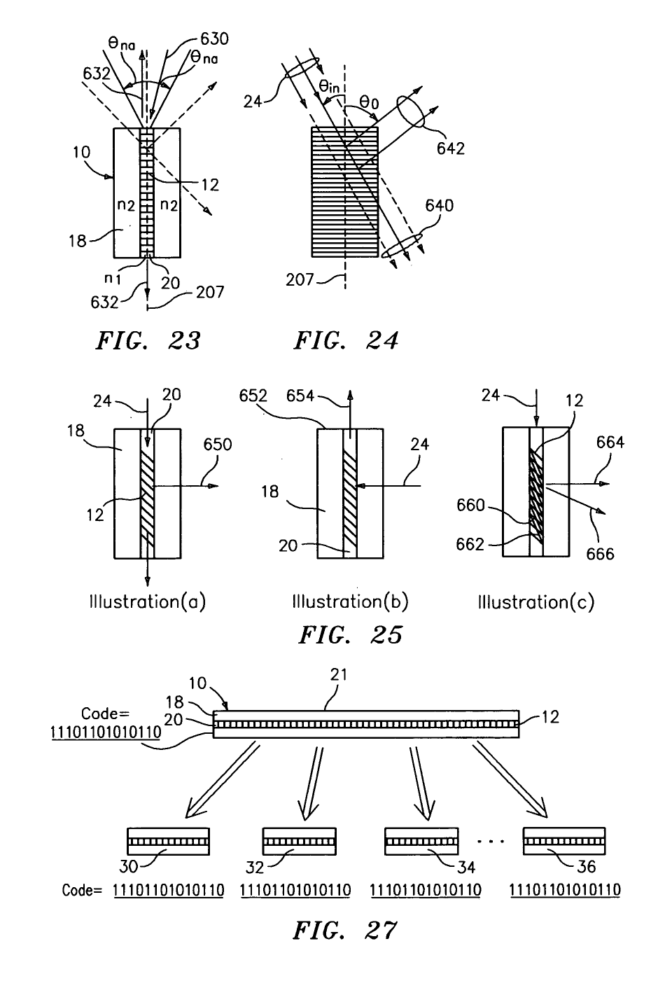

Referring to FIG. 23, if the value of n1 in the grating region 20 is greater than the value of n2 in the non-grating region 18, the grating region 20 of the substrate 10 will act as a known optical waveguide for certain wavelengths. In that case, the grating region 20 acts as a “core” along which light is guided and the outer region 18 acts as a “cladding” which helps confine or guide the light. Also, such a waveguide will have a known “numerical aperture” (θna) that will allow light that is within the aperture θna to be directed or guided along the grating axis 207 and reflected axially off the grating 12 and returned and guided along the waveguide. In that case, the grating 12 will reflect light having the appropriate wavelengths equal to the pitches Λ present in the grating 12 back along the region 20 (or core) of the waveguide, and pass the remaining wavelengths of light as the light 632. Thus, having the grating region 20 act as an optical waveguide for wavelengths reflected by the grating 12 allows incident light that is not aligned exactly with the grating axis 207 to be guided along and aligned with the grating 12 axis 207 for optimal grating reflection.

If an optical waveguide is used any standard waveguide may be used, e.g., a standard telecommunication single mode optical fiber (125 micron diameter or 80 micron diameter fiber with about a 8-10 micron diameter), or a larger diameter waveguide (greater than 0.5 mm diameter), such as is describe in U.S. patent application Ser. No. 09/455,868, filed Dec. 6, 1999, entitled “Large Diameter Waveguide, Grating”. Further, any type of optical waveguide may be used for the optical substrate 10, such as, a multi-mode, birefringent, polarization maintaining, polarizing, multi-core, multi-cladding, or microsturctured optical waveguide, or a flat or planar waveguide (where the waveguide is rectangular shaped), or other waveguides.

Referring to FIG. 24, if the grating 12 extends across the entire dimension D of the substrate, the substrate 10 does not behave as a waveguide for the incident or reflected light and the incident light 24 will be diffracted (or reflected) as indicated by lines 642, and the codes detected as discussed hereinbefore for the end-incidence condition discussed hereinbefore with FIG. 45, and the remaining light 640 passes straight through.

Referring to FIG. 25, illustrations (a)-(c), in illustration (a), for the end illumination condition, if a blazed or angled grating is used, as discussed hereinbefore, the input light 24 is coupled out of the substrate 10 at a known angle as shown by a line 650. Referring to FIG. 25, illustration (b), alternatively, the input light 24 may be incident from the side and, if the grating 12 has the appropriate blaze angle, the reflected light will exit from the end face 652 as indicated by a line 654. Referring to FIG. 25, illustration (c), the grating 12 may have a plurality of different pitch angles 660,662, which reflect the input light 24 to different output angles as indicated by lines 664, 666. This provides another level of multiplexing (spatially) additional codes, if desired.

The grating 12 may be impressed in the substrate 10 by any technique for writing, impressed, embedded, imprinted, or otherwise forming a diffraction grating in the volume of or on a surface of a substrate 10. Examples of some known techniques are described in U.S. Pat. No. 4,725,110 and 4,807,950, entitled “Method for Impressing Gratings Within Fiber Optics”, to Glenn et al; and U.S. Pat. No. 5,388,173, entitled “Method and Apparatus for Forming Aperiodic Gratings in Optical Fibers”, to Glenn, respectively, and U.S. Pat. No. 5,367,588, entitled “Method of Fabricating Bragg Gratings Using a Silica Glass Phase Grating Mask and Mask Used by Same”, to Hill, and U.S. Pat. No. 3,916,182, entitled “Periodic Dielectric Waveguide Filter”, Dabby et al, and U.S. Pat. No. 3,891,302, entitled “Method of Filtering Modes in Optical Waveguides”, to Dabby et al, which are all incorporated herein by reference to the extent necessary to understand the present invention.

Alternatively, instead of the grating 12 being impressed within the substrate material, the grating 12 may be partially or totally created by etching or otherwise altering the outer surface geometry of the substrate to create a corrugated or varying surface geometry of the substrate, such as is described in U.S. Pat. No. 3,891,302, entitled “Method of Filtering Modes in Optical Waveguides”, to Dabby et al, which is incorporated herein by reference to the extent necessary to understand the present invention, provided the resultant optical refractive profile for the desired code is created.

Further, alternatively, the grating 12 may be made by depositing dielectric layers onto the substrate, similar to the way a known thin film filter is created, so as to create the desired resultant optical refractive profile for the desired code.

The substrate 10 (and/or the element 8) may have end-view cross-sectional shapes other than circular, such as square, rectangular, elliptical, clam-shell, D-shaped, or other shapes, and may have side-view sectional shapes other than rectangular, such as circular, square, elliptical, clam-shell, D-shaped, or other shapes. Also, 3 D geometries other than a cylinder may be used, such as a sphere, a cube, a pyramid or any other 3 D shape. Alternatively, the substrate 10 may have a geometry that is a combination of one or more of the foregoing shapes.

The shape of the element 8 and the size of the incident beam may be made to minimize any end scatter off the end face(s) of the element 8, as is discussed herein and/or in the aforementioned patent application. Accordingly, to minimize such scatter, the incident beam 24 may be oval shaped where the narrow portion of the oval is smaller than the diameter D1, and the long portion of the oval is smaller than the length L of the element 8. Alternatively, the shape of the end faces may be rounded or other shapes or may be coated with an antireflective coating.

It should be understood that the size of any given dimension for the region 20 of the grating 12 may be less than any corresponding dimension of the substrate 10. For example, if the grating 12 has dimensions of length Lg, depth Dg, and width Wg, and the substrate 12 has different dimensions of length L, depth D, and width W, the dimensions of the grating 12 may be less than that of the substrate 12. Thus, the grating 12, may be embedded within or part of a much larger substrate 12. Also, the element 8 may be embedded or formed in or on a larger object for identification of the object.

The dimensions, geometries, materials, and material properties of the substrate 10 are selected such that the desired optical and material properties are met for a given application. The resolution and range for the optical codes are scalable by controlling these parameters as discussed herein and/or in the aforementioned patent application.

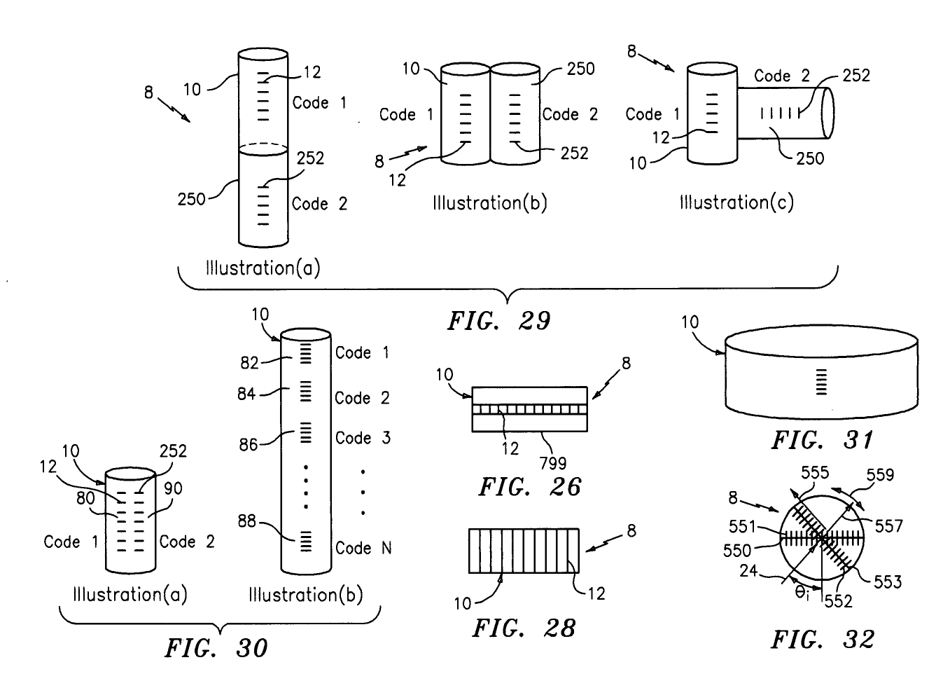

Referring to FIG. 26, the substrate 10 may have an outer coating 799, such as a polymer or other material that may be dissimilar to the material of the substrate 10, provided that the coating 799 on at least a portion of the substrate, allows sufficient light to pass through the substrate for adequate optical detection of the code. The coating 799 may be on any one or more sides of the substrate 10. Also, the coating 799 may be a material that causes the element 8 to float or sink in certain fluids (liquid and/or gas) solutions.

Also, the substrate 10 may be made of a material that is less dense than certain fluid (liquids and/or gas) solutions, thereby allowing the elements 8 to float or be buoyant or partially buoyant. Also, the substrate may be made of a porous material, such as controlled pore glass (CPG) or other porous material, which may also reduce the density of the element 8 and may make the element 8 buoyant or partially-buoyant in certain fluids.

Referring to FIG. 27, the grating 12 is axially spatially invariant. As a result, the substrate 10 with the grating 12 (shown as a long substrate 21) may be axially subdivided or cut into many separate smaller substrates 30-36 and each substrate 30-36 will contain the same code as the longer substrate 21 had before it was cut. The limit on the size of the smaller substrates 30-36 is based on design and performance factors discussed herein and/or in the aforementioned patent application.

Referring to FIG. 28, one purpose of the outer region 18 (or region without the grating 12) of the substrate 10 is to provide mechanical or structural support for the inner grating region 20. Accordingly, the entire substrate 10 may comprise the grating 12, if desired. Alternatively, the support portion may be completely or partially beneath, above, or along one or more sides of the grating region 20, such as in a planar geometry, or a D-shaped geometry, or other geometries, as described herein and/or in the aforementioned patent application. The non-grating portion 18 of the substrate 10 may be used for other purposes as well, such as optical lensing effects or other effects (discussed herein or in the aforementioned patent application). Also, the end faces of the substrate 10 need not be perpendicular to the sides or parallel to each other. However, for applications where the elements 8 are stacked end-to-end, the packing density may be optimized if the end faces are perpendicular to the sides.

Referring to FIG. 29, illustrations (a)-(c), two or more substrates 10,250, each having at least one grating therein, may be attached together to form the element 8, e.g., by an adhesive, fusing or other attachment techniques. In that case, the gratings 12,252 may have the same or different codes.

Referring to FIG. 30, illustrations (a) and (b), the substrate 10 may have multiple regions 80,90 and one or more of these regions may have gratings in them. For example, there may be gratings 12,252 side-by-side (illustration (a)), or there may be gratings 82-88, spaced end-to-end (illustration (b)) in the substrate 10.

Referring to FIG. 31, the length L of the element 8 may be shorter than its diameter D, thus, having a geometry such as a plug, puck, wafer, disc or plate.

Referring to FIG. 32, to facilitate proper alignment of the grating axis with the angle θi of the input beam 24, the substrate 10 may have a plurality of the gratings 12 having the same codes written therein at numerous different angular or rotational (or azimuthal) positions of the substrate 10. In particular, two gratings 550, 552, having axial grating axes 551, 553, respectively may have a common central (or pivot or rotational) point where the two axes 551,553 intersect. The angle θi of the incident light 24 is aligned properly with the grating 550 and is not aligned with the grating 552, such that output light 555 is reflected off the grating 550 and light 557 passes through the grating 550 as discussed herein. If the element 8 is rotated as shown by the arrows 559, the angle θi of incident light 24 will become aligned properly with the grating 552 and not aligned with the grating 550 such that output light 555 is reflected off the grating 552 and light 557 passes through the grating 552. When multiple gratings are located in this rotational orientation, the bead may be rotated as indicated by a line 559 and there may be many angular positions that will provide correct (or optimal) incident input angles θi to the grating. While this example shows a circular cross-section, this technique may be used with any shape cross-section.

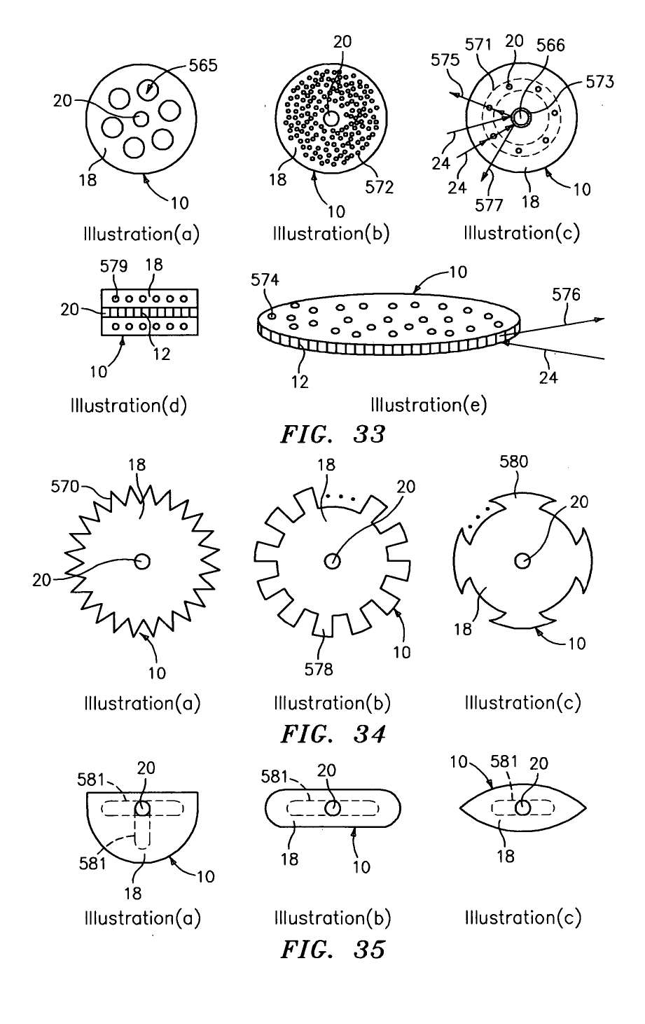

Referring to FIG. 33, illustrations (a), (b), (c), (d), and (e) the substrate 10 may have one or more holes located within the substrate 10. In illustration (a), holes 560 may be located at various points along all or a portion of the length of the substrate 10. The holes need not pass all the way through the substrate 10. Any number, size and spacing for the holes 560 may be used if desired. In illustration (b), holes 572 may be located very close together to form a honeycomb-like area of all or a portion of the cross-section. In illustration (c), one (or more) inner hole 566 may be located in the center of the substrate 10 or anywhere inside of where the grating region(s) 20 are located. The inner hole 566 may be coated with a reflective coating 573 to reflect light to facilitate reading of one or more of the gratings 12 and/or to reflect light diffracted off one or more of the gratings 12. The incident light 24 may reflect off the grating 12 in the region 20 and then reflect off the surface 573 to provide output light 577. Alternatively, the incident light 24 may reflect off the surface 573, then reflect off the grating 12 and provide the output light 575. In that case the grating region 20 may run axially or circumferentially 571 around the substrate 10. In illustration (d), the holes 579 may be located circumferentially around the grating region 20 or transversely across the substrate 10. In illustration (e), the grating 12 may be located circumferentially around the outside of the substrate 10, and there may be holes 574 inside the substrate 10.

Referring to FIG. 34, illustrations (a), (b), and (c), the substrate 10 may have one or more protruding portions or teeth 570, 578,580 extending radially and/or circumferentially from the substrate 10. Alternatively, the teeth 570, 578,580 may have any other desired shape.

Referring to FIG. 35, illustrations (a), (b), (c) a D-shaped substrate, a flat-sided substrate and an eye-shaped (or clam-shell or teardrop shaped) substrate 10, respectively, are shown. Also, the grating region 20 may have end cross-sectional shapes other than circular and may have side cross-sectional shapes other than rectangular, such as any of the geometries described herein for the substrate 10. For example, the grating region 20 may have a oval cross-sectional shape as shown by dashed lines 581, which may be oriented in a desired direction, consistent with the teachings herein. Any other geometries for the substrate 10 or the grating region 20 may be used if desired, as described herein.

Referring to FIG. 36, at least a portion of a side of the substrate 10 may be coated with a reflective coating to allow incident light 510 to be reflected back to the same side from which the incident light came, as indicated by reflected light 512.

Referring to FIG. 37, illustrations (a) and (b), alternatively, the substrate 10 can be electrically and/or magnetically polarized, by a dopant or coating, which may be used to ease handling and/or alignment or orientation of the substrate 10 and/or the grating 12, or used for other purposes. Alternatively, the bead may be coated with conductive material, e.g., metal coating on the inside of a holy substrate, or metallic dopant inside the substrate. In these cases, such materials can cause the substrate 10 to align in an electric or magnetic field. Alternatively, the substrate can be doped with an element or compound that fluoresces or glows under appropriate illumination, e.g., a rare earth dopant, such as Erbium, or other rare earth dopant or fluorescent or luminescent molecule. In that case, such fluorescence or luminescence may aid in locating and/or aligning substrates.

The dimensions and geometries for any of the embodiments described herein are merely for illustrative purposes and, as such, any other dimensions may be used if desired, depending on the application, size, performance, manufacturing requirements, or other factors, in view of the teachings herein.

It should be understood that, unless stated otherwise herein, any of the features, characteristics, alternatives or modifications described regarding a particular embodiment herein may also be applied, used, or incorporated with any other embodiment described herein. Also, the drawings herein are not drawn to scale.

### EXAMPLES

### Example 1

### Specificity of Detection of Hybridization by Coded Beads

An assay was performed with cylindrically shaped glass beads, having dimensions of about 450 microns by 65 microns, using 9 different bead codes, with about 20 to 30 beads of each code. Four different oligonucleotide probes, Probe #1, Probe #2, Probe #3, Probe #4, were bound to four different beads whose codes were 1106, 2090, 8740, and 4424, respectively (see Table 1). The five remaining bead codes, 682, 2470, 2389, 2454, and 618, did not have a DNA probe bound thereto and were used as a control in the assay. Table 1 below shows the bead codes, the DNA probe sequence and Probe # bound to the bead and the melt temperature (Tm) of each DNA probe, which provides relative hybridization strength with respect to Probe #1. Probes #1-4 were randomly selected to provide a variety of different melt temperatures, and thus varying amounts of binding affinity strength difference between the four DNA Probes.

The four 26-nt DNA probe molecules were directly synthesized on the respective coded beads shown in Table 1 using standard phosphoramidite chemistry (referred to as in situ synthesis) with no post synthetic purification. The beads were first derivatized with 18-O-dimethoxytritylhexaethyleneglycol, 1-[(2-cyanoethyl)-(N,N-diisopropyl)]-phosphoramidite to provide a spacer 18 atoms in length (Spacer 18) using standard linker chemistry and ending with a reactive phosphoramidite. Then, the oligo probes were grown base-by-base to create the respective oligonucleotide sequences. (Alternatively, the entire desired oligo sequence may be pre-synthesized and then bound to the bead after completion of the synthesis.)

Then the beads were placed in a blocker solution of Bovine Serum Albumin (BSA; or any other suitable blocker) to prevent non-specific binding of the target polynucleotides.

TABLE 1

| | | | | |
|---|---|---|---|---|
| Bead | | | | |
| Code | DNA Probe Sequence and Linkage to Bead | Probe # | Tm(C) | SEQ ID NO: |

| | | | | |
|---|---|---|---|---|
| 1106 | 5′-GCGTTTTACAATAACTTCTCGTGCCA-3′-Spacer18-Bead | 1 | 66.05 | 1 |
| 8740 | 5′-GCGTTATAGATTAACCTCTCCTGCCA-3′-Spacer18-Bead | 2 | 34.55 | 2 |
| 4424 | 5′-TCAAAATACCATTGCAGCTACCATTT-3′-Spacer18-Bead | 3 | -1.85 | 3 |
| 2090 | 5′-GTGCGTTTTACAATAACTTCCGTGCG-3′-Spacer18-Bead | 4 | 55.35 | 4 |
| 682 | None (Control) | N/A | N/A | |
| 2470 | None (Control) | N/A | N/A | |
| 2389 | None (Control) | N/A | N/A | |
| 6454 | None (Control) | N/A | N/A | |
| 618 | None (Control) | N/A | N/A | |

N/A: Not applicable.

The beads were then hybridized by placing the beads in 5× concentration of SSC (Standard Saline Citrate), 25% formamide, 0.1% SDS (Sodium Dodecyl Sulfate), 20 nanomolar (nM) of a DNA including a sequence complementary to the sequence of Probe #1 and tagged with Cy3 fluorescent dye (Amersham Biosciences/GE Healthcare, Piscataway, N.J.), and 20 nM of the same complementary DNA tagged with Cy5 fluorescent dye (Amersham Biosciences). A total of about 50 microliters of the above hybridization solution was used.

The Cy3- and Cy5-labeled molecules are the target molecules for the assay and are designed to be the complement to the sequence of Probe # 1. The other Probes #2, #3, and #4 are designed to provide varying levels of binding affinity to a target that would bind strongly to Probe #1. In particular, Probe #4 (code 2090) was designed to be only slightly different from Probe #1 (close melt temperature 55.35 deg. C. to that of Probe # 1), Probe #2 (code 8740) was designed to be even more different from Probe #1 and thus have a melt temp. lower than Probe #2, 34.55 deg. C., and Probe #3 (code 4424) was designed to have a sequence much different from Probe #1, and thus a very low melt temp. −1.85 deg. C.

The hybridization was then performed at 50 deg. C. for a period of about 5 minutes. Then, the beads were washed first in a solution of 1×SSC and then in 0.2×SSC.

The results are represented in FIG. 39, which shows the intensity of the Cy3 and Cy5 fluorescence (logarithmically, in counts) for each of the eight bead codes. The graph shows that bead number 1106, which had Probe #1 bound, had the highest level of fluorescence, confirming the expected result that the target molecules, which were intended to be the complementary to, and thus have the strongest affinity to Probe #1. Bead number 2090 had Probe #4 bound thereto and exhibited slightly lower fluorescence level than for Probe #1, confirming the intended result that it had a slight mismatch from Probe #1. Bead numbers 8740 (Probe #2) and 4424 (Probe #3) both exhibited significantly lower fluorescence levels than the level of Probe #1, confirming that they both had very significant mismatches from target complementary to Probe #1. Finally, the control beads having codes 682, 2470, 2389, 2454, and 618, all show a background fluorescence level below all the beads having probes bound. Thus, this Example shows that the codes can be read with the probes and the fluorescent dyes being bound to the beads. For each bead there will be a Cy3 (green) and a Cy5 (red) fluorescence (i.e., a red-green fluorescent data pair).

In addition, the data shows that each bead may have slightly different fluorescence intensity or count level. Because the beads allow for a high number (greater than 50 million) codes, if desired, each bead may be labeled, even ones that have the same probe attached. This would allow for evaluation of signals from each bead, thereby allowing better quality control of the data provided. For example, the graph for code 1106 has a red-green pair of points (probably from the same bead) that is much lower level than the other points on the graph. If each bead was labeled with a separate unique code, one could know exactly which bead exhibited this characteristic, and the bead could then be re-analyzed to determine if there was a problem with the chemistry on the bead, e.g., that the bead did not have the correct Probe put on it. This allows for quality control to be performed on the beads to enhance data credibility and accountability.

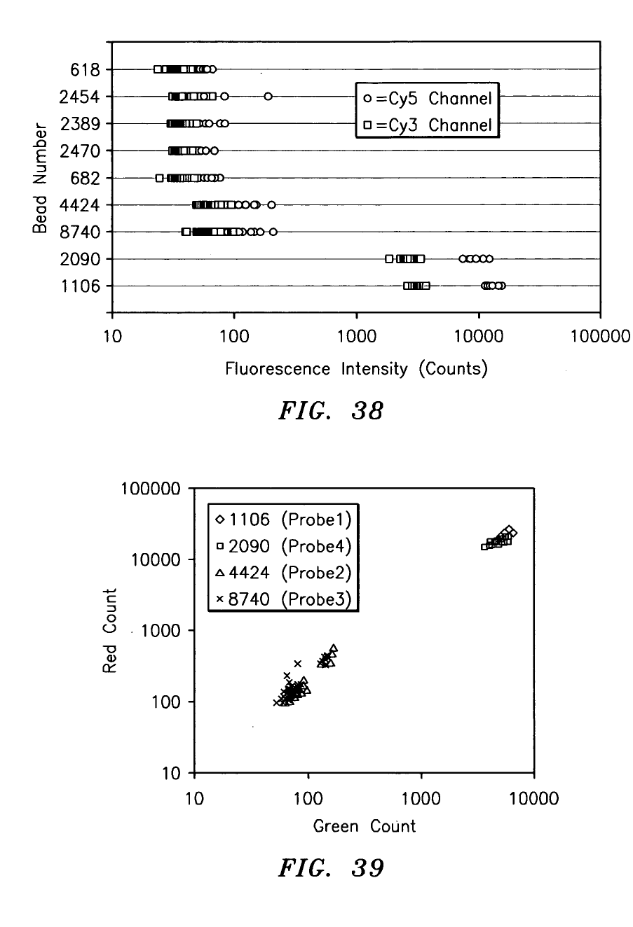

In FIG. 39, the data in FIG. 38 are shown as the red Cy5 intensity against the green Cy3 intensity in a log-log plot. This graph illustrates that the targets labeled with Cy3 and Cy5 are uniformly distributed on the beads. When the points fall along a straight line, the distribution of red and greed molecules on each of the beads is substantially uniform, as found in FIG. 39. Accordingly, the assay of the present invention show high uniformity. Further, as discussed before, if there are points that fall outside the desired field for quality data, had each of the beads been labeled with a unique code, those beads could be re-examined to further evaluate the anomaly.

### Example 2

### Labeled Oligonucleotide Sample Assays

A set of 67-nt oligonucleotide-bearing particles was synthesized in situ on coded particles. The probes are shown in Table 3. These particles were hybridized with the 5′-Cy3-labeled 67-nt complement to the phix310s probe shown in Table 2.

TABLE 2

| | | | |
|---|---|---|---|
| | | | SEQ |
| | | | ID |
| Code | Probe Name | Probe Sequence | NO. |

| | | | |
|---|---|---|---|
| 342 | PhiX310s | GCCCTGGTCGTCCGCAGCCGTTGCGAGG | 5 |
| | | TACTAAAGGCAAGCGTAAAGGCGCTCGTC | |
| | | TTTGGTATG | |
| 345 | PhiX310as | CATACCAAAGACGAGCGCCTTTACGCTTG | 6 |
| | | CCTTTAGTACCTCGCAACGGCTGCGGACG | |
| | | ACCAGGGC | |
| 346 | PhiX604s | ATTAGCATAAGCAGCTTGCAGACCCATAAT | 7 |
| | | GTCAATAGATGTGGTAGAAGTCGTCATTT | |
| | | GGCGAGAA | |
| 357 | PhiX604as | TTCTCGCCAAATGACGACTTCTACCACATC | 8 |
| | | TATTGACATTATGGGTCTGCAAGCTGCTTA | |
| | | TGCTAAT | |
| 358 | PhiX1072s | CATTTCCTGAGCTTAATGCTTGGGAGCGT | 9 |
| | | GCTGGTGCTGATGCTTCCTCTGCTGGTAT | |
| | | GGTTGACG | |
| 5541 | PhiX1072as | CAAGTATCGGCAACAGCTTTATCAATACC | 10 |
| | | TGAAAAATATCAACCACACCAGAAGCAGC | |
| | | ATCAGTGA | |
| 5546 | PhiX1353s | GCGCGGTAGGTTTTCTGCTTAGGAGTTTA | 11 |
| | | ATCATGTTTCAGACTTTTATTTCTCGCCAT | |
| | | AATTCAAA | |
| 8789 | PhiX1353as | GAGAAATAAAAGTCTGAAACATGATTAAAC | 12 |
| | | TCCTAAGCAGAAAACCTACCGCGCTTCGC | |
| | | TTGGTCAA | |

The hybridization buffer was 25% formamide, 0.1% SDS, and 5×SSC. The '310s oligo complement was prepared at 1 nM concentration and a 50 microliter total hybridization volume was used. Hybridizations were performed at approximately 42 degrees C for 1 hour. Particles were washed in 1×SSC three times and 1×SSC and 0.1% SDS once, then scanned for bead code and Cy3 signal intensity.

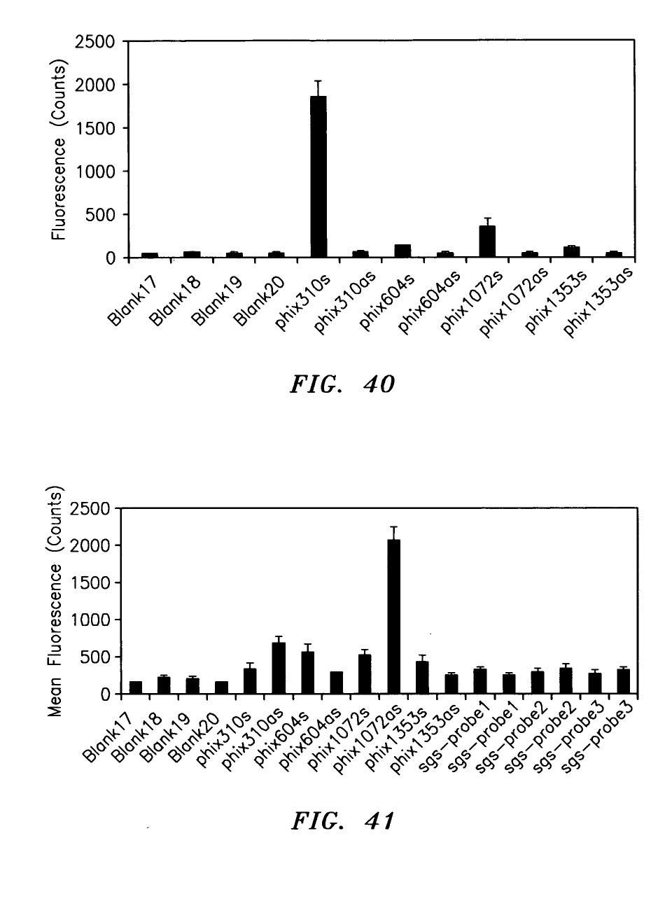

FIG. 40 and Table 3 show the results. In Table 3, G is the signal intensity, and % CV is the percent coefficient of variation. It is seen that, as expected, only

TABLE 3

| | No. of | | | |
|---|---|---|---|---|
| ID | Beads | G Mean | G StdDev | G % CV |
| Blank17 | 5 | 47 | 4 | 8.52 |
| Blank18 | 8 | 53 | 8.5 | 16.09 |
| Blank19 | 8 | 51 | 10.2 | 19.92 |
| Blank20 | 13 | 48 | 10.8 | 22.36 |
| phix310s | 15 | 1849 | 179.5 | 9.71 |
| phix310as | 8 | 58 | 8 | 13.86 |
| phix604s | 2 | 129 | 9.4 | 7.29 |
| phix604as | 8 | 50 | 9.9 | 19.59 |
| phix1072s | 5 | 350 | 94.8 | 27.1 |
| phix1072as | 17 | 49 | 6.8 | 13.87 |
| phix1353s | 10 | 100 | 23.1 | 23.16 |
| phix1353as | 14 | 50 | 8.6 | 17.08 |

the bead bearing the phix310s probe exhibits significant fluorescence above background. In addition, it is important that, as seen in Table 3, a sample number of particles as low as 2, or 5, provides standard deviations and percent coefficients of variation that are significant, and comparable to those obtained with larger numbers of particles.

### Example 3

### Detection and Quantitation of a Target Nucleic Acid in a Sample Containing a cDNA Library

Referring to FIG. 40, a biological assay was performed with the cylindrically shaped encoded glass microbeads described herein, having synthesized probes attached to the beads seeking a natural biological target analyte.

Each probe was bound to a particle having a unique code. There were 8 different PhiX174 DNA oligonucleotide probes obtained that were complementary to 8 different PhiX174 DNA restriction fragments designated phix310s, phix310as, phix604s, phix604as, phix1072s, phix1072as, phix1353s, and phix1353 as (where s=sense and as=antisense). The fragments were isolated as follows. (1) Four HaeIII digested fragments from bacteriophage PhiX174 were obtained from gel extraction. The fragment lengths are 310, 604, 1072, 1353 bases long respectively (hence the naming of the probes). (2) The fragments were blunt-end ligated into Sma1-digested pSP64 polyA cloning vectors (Promega Corp., Madison, Wis.). (3) The resulting cloning vectors were used for in-vitro transcription (IVT) of RNA transcripts via the SP6 promoter site on the pSP64 cloning vector. (4) The resulting RNA transcripts were spiked into the HeLa samples as non-interfering controls.

The PhiX probes (given in the Table for Example 2) were oligonucleotides synthesized in situ. Also, other “sgs” or “standard-gene-set”-type oligo probes were used, e.g., sgs-probe1, sgs-probe2, sgs-probe3. In this case, beads with 2 different codes were used for each of these probes. The sgs-type probes were pre-synthesized and then attached to the beads. Table 4 shows the sequence for the 3 sgs probes.

TABLE 4

| Probe | | SEQ ID |
|---|---|---|
| Name | Probe Sequence | NO: |
| SGS1 | 5′-amine- | 13 |
| | catccgacattgaagttgacttactgaagaatggagagagaattgaaaaagtg | |
| | gagcattcagacttgtc-3′ | |
| SGS-2 | 5′-amine- | 14 |
| | atgtcgcggtttttcaccaccggttcggacagcgagtccgagtcgtccttgtccg | |
| | gggaggagctcgtca-3′ | |
| SGS-3 | 5′-amine- | 15 |
| | agagaacttcaaaaaaccaactagaagcaacatgcagagaagtaaaatga | |
| | gaggggcctcctcaggaaag -3′ | |

In addition, a series of blank encoded beads (e.g., Blank17 through Blank20) having no oligo probes attached (i.e., bare glass beads) were used.

The biological target analyte samples were total RNA obtained from 20 ug of HeLa human biological cell line. The HeLa RNA was combined with 1 ng of an In-Vitro Transcription-generated (IVT) fragment of PhiX174 RNA designated “PhiX1072”, and the mixture was reverse transcribed and labeled with Cy3. This yielded approximately 2 ug of Cy3 labeled cDNA mixture of the HeLa cDNA library and PhiX1072. Then, these 2 micrograms of Cy3-cDNA HeLa and Cy3-PhiX1072 were resuspended into 20 microliters of water, referred to herein as the “cDNA Aqueous Solution”, having approximately 300 attomoles of Cy3 labeled cDNA “PhiX1072” spike.

Approximately 10-20 beads of a given code were used for each probe and put in a “hybridization buffer solution” of: about 20% Formamide, about 0.1% SDS (sodium dodecyl sulfate), and about 5×SSC. The hybridization buffer solution was then removed from the beads (which settled to bottom) and 10 microliters of the cDNA Aqueous Solution (discussed above) was added to beads in a tube. The beads were then dried by centrifuging under a vacuum. Then, 10 microliters of the hybridization buffer solution (described above) was added, the beads were heated in boiling water for about 5 minutes, quenched into ice for about 2 minutes, and incubated at about 42 Deg. C. for about 16 hours to perform the hybridization.

After hybridization, the beads were washed 3 times with 1×SSC, then added 0.1% SDS into 1×SSC solution with the beads. Then the beads were loaded onto a CyVera Corp. groove plate for reading of the bead codes and the fluorescence intensity.

The results are shown for each bead in FIG. 41. It is seen that only the beads having the “phix1072as” probe exhibit significant fluorescence over the background of the three blank bead samples. This Example demonstrates that coded beads with a variety of probes have the ability to provide, in the real time during which the beads are analyzed in the reader, both the code, thereby identifying the probe bound to the bead, as well as the ability to bind to the specific target when it is present in a background of a high concentration of nonspecific DNA.

The data are also presented in Table 5. It is seen that a sample number of beads as low as 3, or 5, provides standard deviations and percent coefficients of variation that are significant, and comparable to those obtained with larger numbers of beads. Even control samples with only 1 bead (Blank17 and Blank20) provide results not in exception from control samples with several beads (Blank18 and Blank19).

TABLE 5

| | | | Mean | |
|---|---|---|---|---|
| | | | Fluorescence | |
| Code | ID | No. of Beads | (Counts) | % CV |
| 2389 | Blank17 | 1 | 180 | N/A |
| 2390 | Blank18 | 5 | 232 | 11.25 |
| 2393 | Blank19 | 3 | 219 | 13.23 |
| 2394 | Blank20 | 1 | 173 | N/A |
| 342 | phix310s | 33 | 357 | 17.93 |
| 345 | phix310as | 5 | 700 | 11.46 |
| 346 | phix604s | 6 | 588 | 16.65 |
| 357 | phix604as | 6 | 302 | 5.46 |
| 358 | phix1072s | 22 | 522 | 15.53 |
| 5541 | phix1072as | 5 | 2062 | 9.24 |
| 5546 | phix1353s | 22 | 447 | 18 |
| 8789 | phix1353as | 29 | 251 | 14.8 |
| 8362 | sgs-probe1 | 15 | 327 | 13.7 |
| 8358 | sgs-probe1 | 7 | 251 | 17.31 |
| 8363 | sgs-probe2 | 12 | 299 | 15.29 |
| 8359 | sgs-probe2 | 27 | 350 | 15.79 |
| 8364 | sgs-probe3 | 6 | 283 | 16.15 |
| 8357 | sgs-probe3 | 15 | 325 | 13.82 |

### Example 4

### Labeled Oligonucleotide Assays Using 50-nt Oligonucleotide Probes

A set of 50-nt oligonucleotide-bearing particles was prepared by in situ synthesis. The 50-mers were designed to serve as probes for a set of mouse mRNA transcripts indicated in Table 6.

TABLE 6

| | | GenBank | |
|---|---|---|---|
| | Short | Accession | |
| Probe Name | Name | Number | Gene Description |
| Rabbit bGlo- | BGlob | V00882 | Rabbit (O. cuniculus) beta-globin. |
| 50ac | | | |
| Ec16S-50ac | Ec16s | AE016767 | Escherichia coli CFT073 section |
| | | | 13 of 18 of the complete genome |
| Oligo Mouse | aMHC | M74751 | Mouse myosin heavy chain |
| aMHC-50ac | | | mRNA, 3 flank. |
| Oligo Mouse | ANF | K02781 | Mouse PND gene encoding atrial |
| ANF-50ac | | | natriuretic factor, complete CDS. |
| Oligo Mouse | bAct | X03672 | Mouse cytoskeletal mRNA for |
| bACT-50ac | | | beta-actin. |
| Oligo Mouse | bMHC | M38128 | Mouse cardiac myosin heavy chain |
| bMHC-50ac | | | beta isoform mRNA, 3 end. |
| Oligo Mouse | GAPD | M32599 | Mouse glyceraldehyde-3-phosphate |
| GAPD-50ac | | | dehydrogenase mRNA, complete |
| | | | CDS. |
| Oligo Mouse | LC1 | M19436 | Mouse atrial/fetal myosin alkali |
| LC1-50ac | | | light chain (Myla) mRNA, clone |
| | | | pCL10.4. |
| Oligo Mouse | LC2 | NM_010861 | Mus musculus myosin light chain, |
| LC2-50ac | | | phosphorylatable, cardiac |
| | | | ventricles (Mylpc), mRNA. |
| Oligo Mouse | RPL19 | NM_009078 | Mus musculus ribosomal protein |
| RPL19-50ac | | | L19 (Rpl19), mRNA. |
| Oligo Mouse | Ubiq | X51703 | Mouse mRNA for ubiquitin. |
| Ubiquitin- | | | |
| 50ac | | | |

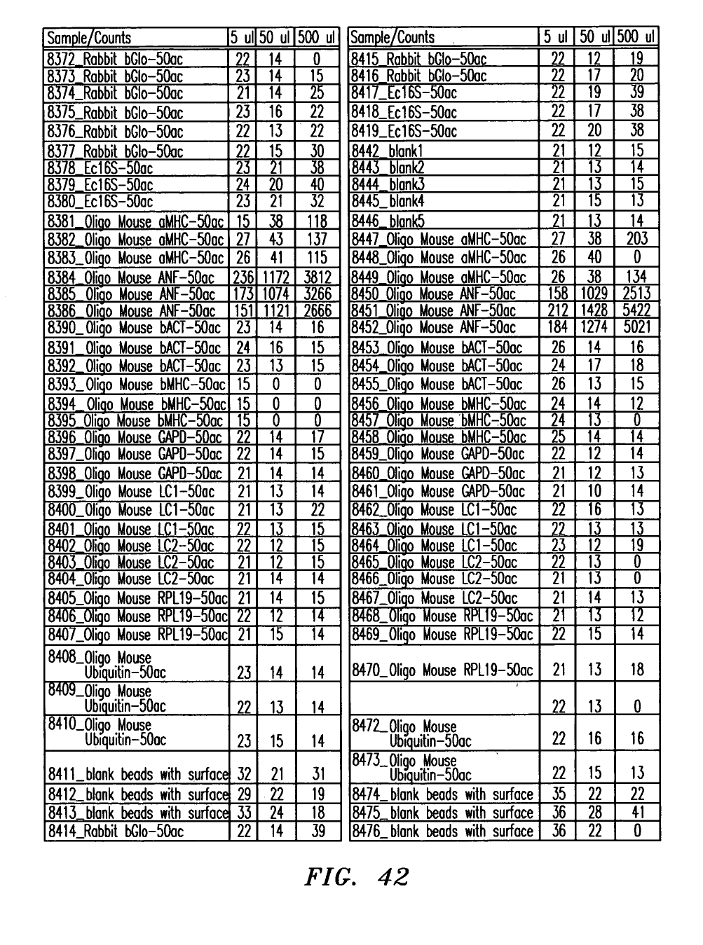

A total of 81 differently-coded particles bearing probes targeting the sequences in Table 6, in sets of 3, as well as blank controls, was prepared and assembled into a single set for analysis. In certain cases different probe sequences for the same target were synthesized. The target sample contained 1 nM concentration of a labeled complement of the ANF transcript. The amounts of target ranged over a 100-fold difference by adding sample volumes of 5, 50, or 500 ul, of the ANF transcript solution. The results are shown for all three sample volumes for all 81 particles in FIG. 42. For each bead label shown, three bars are projected, representing the three volumes of sample added. Although the labels in FIG. 42 may not be legible, the figure shows that only two sets of particles bearing an ANF-directed probe, each set being used in triplicate, become labeled. This Example shows that coded particles contained in a complex mixture of particles with a variety of probes maintains specificity for binding the cognate target.

### Example 5

### Determination of Assay Sensitivity

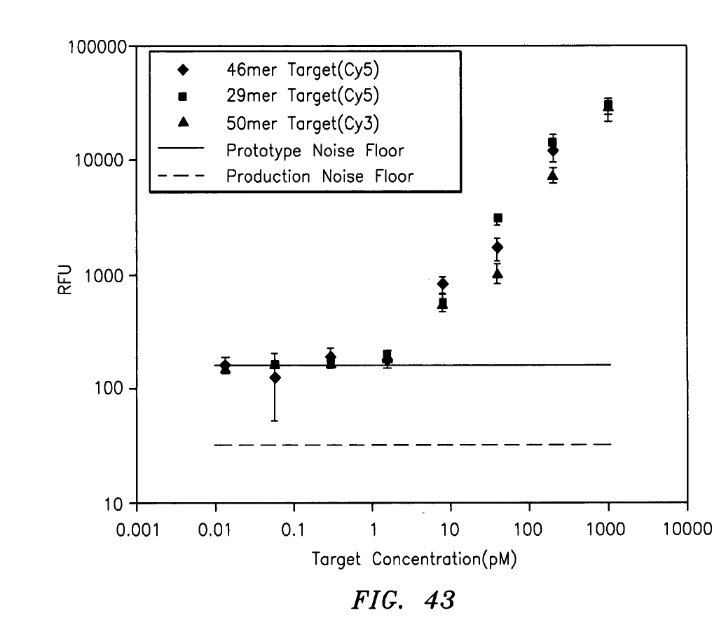

FIG. 43 shows the results of a titration experiment, in a log-log plot, where the concentrations in solution of various labeled complementary probes and Cy3- or Cy5-labeled targets were varied. The 50-nt probes for the ubiquitin and beta globin genes listed in Table 6 were used. Assays were performed independently for each of the three types of dye-labeled oligos described below. Each assay was run with only a single type of labeled oligo present. Eight assays were performed with a Cy5 labeled 29-mer complementary to the ubiquitin probe at varying concentrations. Seven assays were performed for a Cy5 labeled 46-mer complementary to the rabbit beta globin probe. Eight assays were performed for a Cy3 labeled 50-mer complementary to the rabbit beta globin probe. Each assay was run for 2 hours at 42 C. The “formamide” hybridization buffer (Example 6) was used. Beads were washed 3× in 1×SSC, 1× in 0.1×SSC, and then resuspended and scanned in 1×SSC/0.1% SDS.

The results in FIG. 43 show that, regardless of the probe-target studied, the sensitivity of the assay, defined as providing approximately a signal 2-fold over background, is about 2 pM target, measured in a prototype bead reader. A calculated sensitivity for a bead reader under development by CyVera Corp. is shown in the lower line of FIG. 42. The projected noise level predicts a sensitivity of about 0.1 pM for Cy3 and Cy5 labels. Such sensitivities are highly advantageous in performing a wide variety of diagnostic and biotechnological assays.

### Example 6

### Stability of Probe-Target Pairs over Time in Various Hybridization Buffers

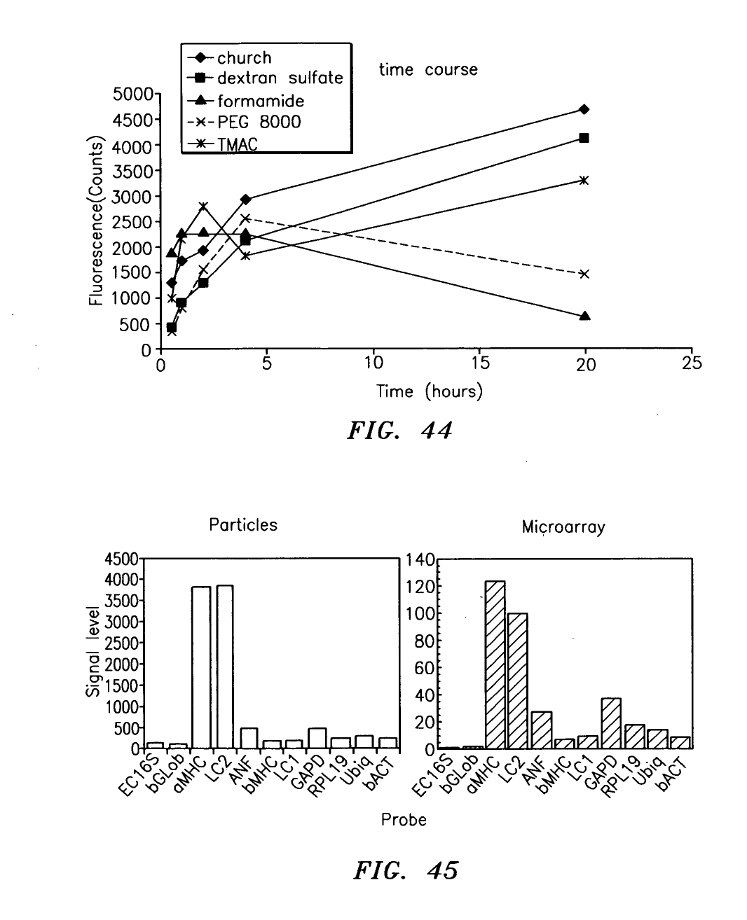

The kinetics of formation of hybridized complexes of probe and target were studied in a variety of hybridization buffers, namely buffers designated “Church”, “Dextran Sulfate”, “Formamide”, “PEG 8000”, and “TMAC”. FIG. 44

Dextran Sulfate

| | |
|---|---|
| 10% | Dextran Sulfate |
| 25% | Formamide |
| 3X | SSC |
| .1% | Tween |
| 5X | Denhardts |
| 500 ug/mL | BSA |
| 100 ug/ml | Herring Sperm |
| | DNA |

PEG 8000

| | |
|---|---|
| 10% | PEG 8000 |
| 20 mM | NaHPO₄ pH 7.2 |
| 25% | Formamide |
| 3X | SSC |
| .1% | SDS |
| 5X | Denhardts |
| 500 ug/mL | BSA |
| 100 ug/ml | Herring Sperm |
| | DNA |

Church

| | |
|---|---|
| 200 mM | NaHPO₄ pH 7.2 |
| 25% | Formamide |
| 7% | SDS |
| 5X | Denhardts |
| 500 ug/mL | BSA |
| 100 ug/ml | Herring Sperm |
| | DNA |

Formamide

| | |
|---|---|
| 25% | Formamide |
| 3X | SSC |
| 0.1% | SDS |
| 5X | Denhardts |
| 500 ug/mL | BSA |
| 100 ug/ml | Herring Sperm |
| | DNA |

TMAC

| | |
|---|---|
| 3 M | TMAC |
| 0.1% | Tween |
| 5X | Denhardts |
| 500 ug/mL | BSA |
| 100 ug/ml | Herring Sperm |
| | DNA |

shows the result of hybridizing 50-mer Cy3-labeled complementary oligonucleotides to particles that had 50-mer oligonucleotides attached. In all cases the hybridization volume was 50 microlitres, the temperature was 42 degrees C., and the labeled oligo concentration was 1 nM. The buffers used in the stability study are listed in the tables above with their compositions. It is shown in FIG. 44 that oligonucleotide probe-target hybridized microparticles develop a high degree of hybridization by 4 hr. The Church, dextran sulfate, and TMAC hybridization buffers promote retention of stability for 24 hours. These buffers may advantageously increase the rate of hybridization in some cases.

### Example 7

### Mouse Cardiac/Rabbit Beta Globin cDNA Assays

A series of biological samples were assayed using particles bearing the same set of proprietary 50-mer mouse and rabbit probes as described in Example 4. Samples of mouse cardiac total RNA was obtained from Ambion Inc. Samples were reverse transcribed into cDNA and applied to a spotted microarray. FIG. 45 (right panel) shows the resulting levels measured on the microarray.

A set of microparticles with the same 11 transcripts as on the microarray were reacted with a similarly prepared sample used in the microarray. Resulting transcription levels measured by the particles are shown in FIG. 45 (left panel). As can be seen the detected levels correlate well. This Example shows that particles bearing specific probe oligonucleotides hybridize to target sequences in a comprehensive cDNA library with efficiency and specificity that is equal to or exceeds that obtained using a microarray.

### Example 8

### Detection of Rabbit Beta Globin Spiked into a Library

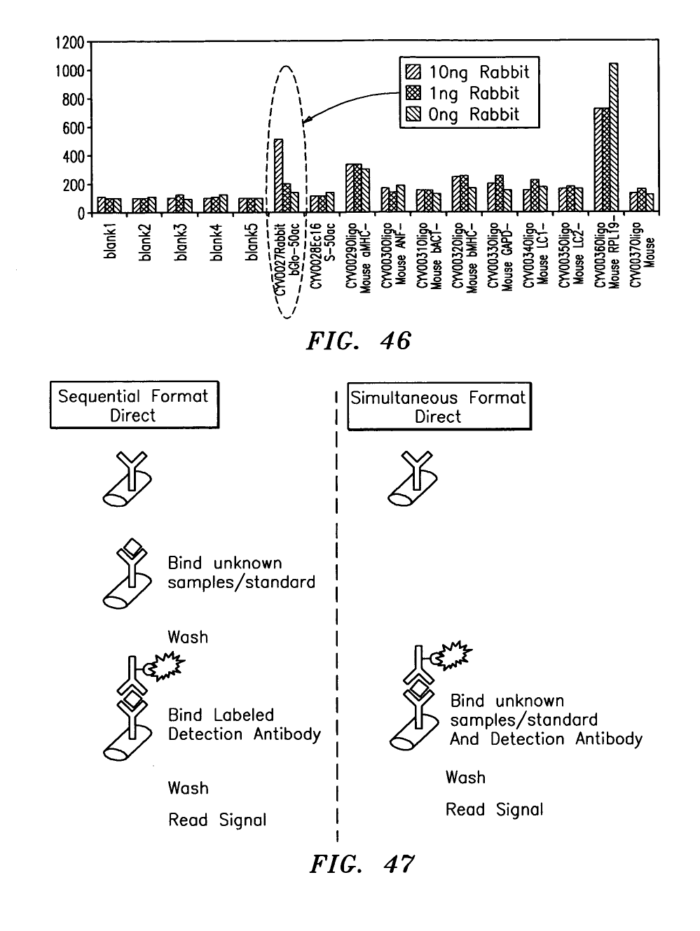

In a separate set of experiments, rabbit beta globin mRNA was spiked into the mouse cardiac RNA before transcription. A different amount (10, 1 and 0 ng respectively) of rabbit beta globin RNA was spiked into the sample before reverse transcription. FIG. 46 shows the resulting transcription levels measured in this manner. As can be seen in the Figure, the rabbit beta globin shows the expected dose-response relationship while most of the other transcripts remain unchanged (RPL19 shows an “outlier” level in one of the assays.)

### Example 9

### Immobilization of Proteins to Beads

Proteins were immobilized or attached to amino terminated microbeads using a cross-linking reagent such as, EDC (1-(3-dimethylaminopropyl)-3-Ethyl-carbodiimide HCL) and Sulfo-NHS(N-Hydroxysulfosuccinimide) chemistry. A number of other suitable chemical cross-linkers may also be used such as BS³ Bis(sulfosuccinimidyl) suberate, SATA (n-Succinimidyl S-acetylthioacetate. In particular, amino terminated microbeads were washed 3 times with 0.1 M MES (2-(N-Morpholino)ethanesulfonic acid), pH 4.5 to remove any residual buffer which may inhibit the reaction. Fifty (50) mg of both EDC and Sulfo-NHS were weighed and diluted with 1.0 milliliter (ml) of 0.1 M MES, pH 4.5 just before addition to microbeads. After removal of residual buffer from microbeads, the sulfo-NHS is resuspended as described above and 250 microliters (ul) of the Sulfo-NHS solution is added to the microbeads. The mixture was vortexed to resuspend microbeads for 2 seconds and 250 ul of the EDC mixture is added. The mixture was vortexed to resuspend microbeads and incubated rocking at ambient temperature for about 60.

The microbeads were then separated and washed once with 0.1 M MES, pH 4.5 to remove excess x-linker. Residual x-linker associated with the amino terminated microbeads is likely used during the x-linking reaction. The excess buffer was removed and the known protein was added. It is important to have the protein free of other proteins or amine based salts which can inhibit or bind during the coupling reaction of the protein of interest. It is recommended that the protein be diluted in 0.1 M Phosphate, 1× Phosphate buffered saline (PBS) or any other suitable buffer at a concentration of between 1-200 ug/ml. The protein was “coupled” via the available carboxyl groups on the protein to the amine terminated microbeads for a minimum of about 60 minutes not to exceed about 16 hours. Longer or shorter incubation time may be used providing sufficient numbers of protein molecules are attached to the beads. The protein coupled microbeads were washed twice with PBS-0.05% Tween-20 (PBST) to remove uncoupled protein. A final wash consists of PBS-1.0 mg/ml of Bovine Serum Albumin (BSA) to remove excess detergent. The beads were then “blocked” in 1.0 ml of PBS-BSA buffer for about an 1 hour at room temperature under gentle agitation sufficient to block potential sites for non-specific binding during the immunoassay.

### Example 10

### Particle-Based Protein Immunoassay

A primary antibody probe is bound to a particle of the invention as described in Example 9. The presence and/or amount of the cognate antigen to which the primary antibody binds is determined as described in this Example, after exposing a sample presumed to contain the antigen to the antibody-bearing particles.

A. Directly Labeled Secondary Antibody

The antigen-primary antibody complex bound to the particle is further exposed to a solution of containing a secondary antibody that specifically binds the antigen. The secondary antibody is labeled with a detectable label, such as a fluorescent molecule (see FIG. 47; the label is depicted by the starburst object). The assay may be performed in two steps (FIG. 47, left side), by first probing for the antigen, and then adding the secondary antibody. Alternatively the assay may be performed in one step, by simultaneous addition of the sample and the secondary antibody (FIG. 47, right side). In either case, the formed ternary complex is washed to remove non-specifically bound label and detected in a suitable reader. Quantitation of the unknown sample is done by comparing the fluorescent intensity of the unknown sample to the fluorescent intensity signal of a standard curve.

B. Indirectly Labeled Secondary Antibody

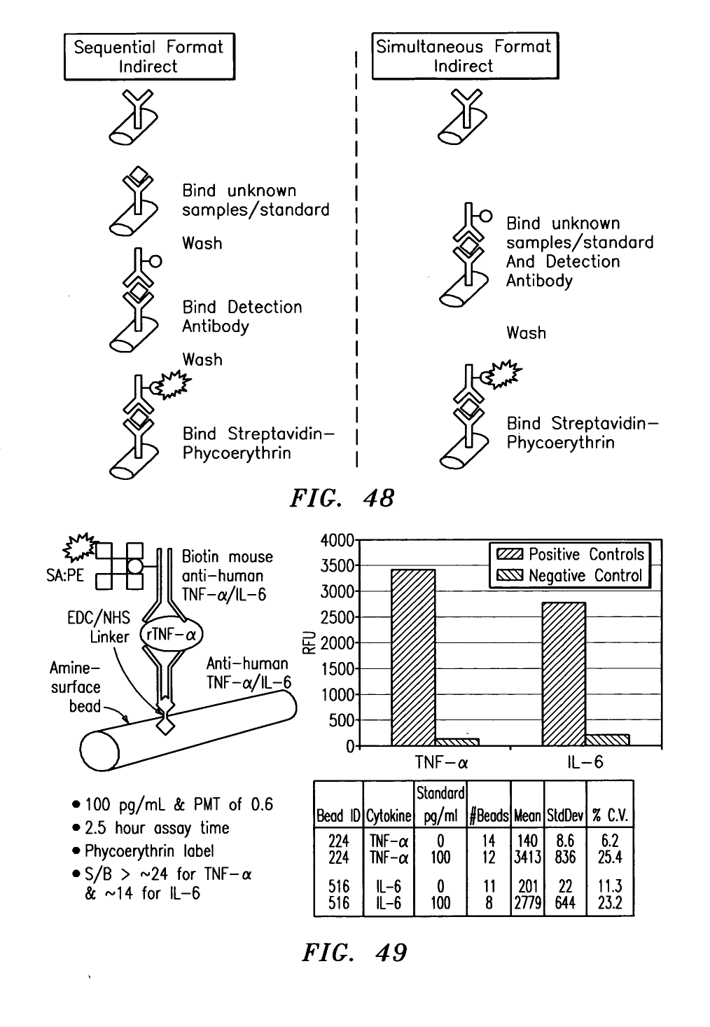

An indirect immunoassay is performed identically to the steps described above in Paragraph A and FIG. 47, except that in this case the secondary antibody carries a molecule that is not directly detectable; it requires binding to an additional substance carrying a label that is detectable (see FIG. 48). As an example, as shown in FIG. 48, the secondary antibody is coupled with biotin, which then further binds to a streptavidin-phycoerythrin conjugate. As with the direct immunoassay, this assay can also be run simultaneously or sequentially as depicted in FIG. 48.

### Example 11

### Immunoassay for a Cytokine

This Example illustrates a multiplexed cytokine assay using the sequential format (Example 10). 500 ul of a multiplex standard containing 100 picograms per ml (pg/ml) of both recombinant tumor necrosis factor-alpha (TNF-a) and recombinant interleukin-6 (IL-6) (both proteins were from R & D Systems, Minneapolis, Minn.) were added to a multiplex particle preparation. The particles were bound to either goat anti-TNF-a antibody or goat anti-IL-6 antibody (R & D Systems) using a linker and EDC-NHS coupling chemistry (Example 9). The particles and target proteins were incubated for 1 hour at ambient temperature under gentle agitation; negative controls were not exposed to the target proteins. Then 3 washes with PBST were performed to remove excess targets. The particles were then incubated with 500 ul of a multiplex detection cocktail for 1 hour at ambient temperature under gentle agitation. This multiplex cocktail contained biotinylated goat anti-TNF-a and goat anti-IL-6 antibodies (R & D Systems) at 500 nanogram per ml (ng/ml) or a concentration suitable to detect all bound target proteins. Three (3) additional washes with PBST were done to remove non-specifically bound detection antibodies. Signal generation is achieved by a 30 minute incubation with streptavidin-phycoerythrin (Molecular Probes, Inc., Eugene, Oreg.) at a concentration of about 2 ug/ml. To remove any non-specifically bound label another 3 washes with PBST were performed. The bound complex(es) were now detected in a fluorescent particle reader (CyVera Corp.). Total time to result for this sequential format was about 2.5 hours.

The results are shown in FIG. 49. The left panel shows a schematic diagram of the final detected particle-antibody-antigen complex. The secondary antibody conjugated with biotin (circle) is bound streptavidin (assembly of five squares arranged to provide the four biotin binding cavities) which itself is conjugated to phycoerythrin (starburst object; SA:PE). The experimental results are shown as relative fluorescence units (RFU) are in the right panel, with statistical data shown in the table below the graph. Significant ratios of signal over background (S/B) are seen for both TNF-a (S/B˜24) and IL-6 (S/B ˜14) recombinant antigen standard at 1000 pg/ml. This Example demonstrates the ability of coded particles of the invention to detect target proteins with high sensitivity in a multiplexed format.

### Example 12

### Sensitivity of Particle-Based Immunoassay

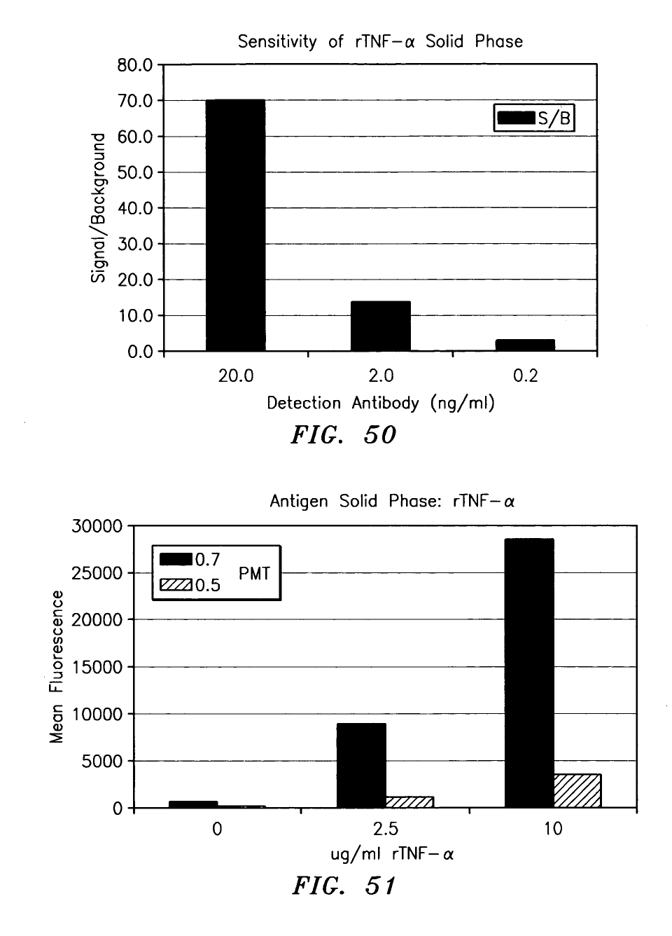

The sensitivity of indirect secondary antibody procedure was determined under conditions likely to be encountered in a clinical setting. Recombinant TNF-a at 10 ug/ml was absorbed overnight in PBS, washed three times with PBST (0.05%), blocked with PBS/BSA (1%) for 1 hour, and washed once with PBS/BSA. For the assay,, biotinylated anti-human IL-6 antibody was titrated from 20 ng/ml to 0.2 ng/ml in PBS plus 10% fetal calf serum (FCS). The secondary antibody was incubated 30 minutes at room temperature, and washed three times with PBST (0.05%). 1.6 ug/ml SA:PE was added and incubated for 30 minutes, washed three times with PBST, and scanned. The results are shown in FIG. 50, which demonstrates the ability to detect 0.2 ng/ml secondary antibody in 10% FCS at a signal/background >2.0.

### Example 13

### Multiplex Sandwich Immunoassay

This example shows the results of a 4 Plex cytokine immunoassay using digitally encoded beads. Briefly, 50 ug/ml of each of four capture antibodies directed against TNF-a, IL-3, IL-6, and IL-8 was immobilized by EDC/NHS chemistry (Example 9) to an individual digitally encoded bead. The individual bead subsets were pooled together and incubated with an antigen pool having a concentration of about 1000 picogram/milliliter (pg/ml). After sufficient time to allow antigen binding to the solid support, the complex was washed to remove excess antigen. The bead-bound antigen was then allowed to react with a pool of biotinylated secondary antibodies for sufficient time as to allow binding, washed, and incubated with phycoerythrin-streptavidin label. The fluorescent complex was then detected and a signal is generated which is proportional to the amount of captured antigen. The results are shown in Table 7, where “S/B” represents “signal to background ratio”. It is seen that a multiplexed immunoassay successfully distinguishes target antigens with high sensitivity.

TABLE 7
4 Plex Cytokine Immunoassay

| | TNF-a | IL-3 | IL-6 | IL-8 |
|---|---|---|---|---|
| S/B | 29.0 | 13.3 | 42.4 | 50.1 |

### Example 14

### Adsorbed Antigen Immunoassays

Various concentrations (20, 2, and 0.2 ug/ml) of recombinant Tumor Necrosis Factor alpha (TNF-a) in phosphate buffered saline (PBS), pH 7.4, were allowed to adsorb to amine functionalized digitally encoded microbeads overnight at 25 C. The antigen microbeads were washed 3 times and blocked for 1 hour with PBS containing 1% bovine serum albumin (PBS/BSA). The antigen-microbeads (Ag-microbeads) were allowed to react with biotinylated anti-TNF-a (75 ng/ml) for 30 minutes, washed to remove excess and non-specifically bound detection antibody. The bound antibody was detected with streptavidin-phycoerythrin for 30 minutes. After washing, the bound complex was detected in a fluorescent scanner. FIG. 51 shows that the signal increases as the amount of immobilized Ag increases to the highest concentration tested. Since the signal generated by the highest concentration (10 ug) exceeded the linear range of the bead reader at PMT 0.7, each sample was rescanned at a lower (0.5 PMT) laser setting.

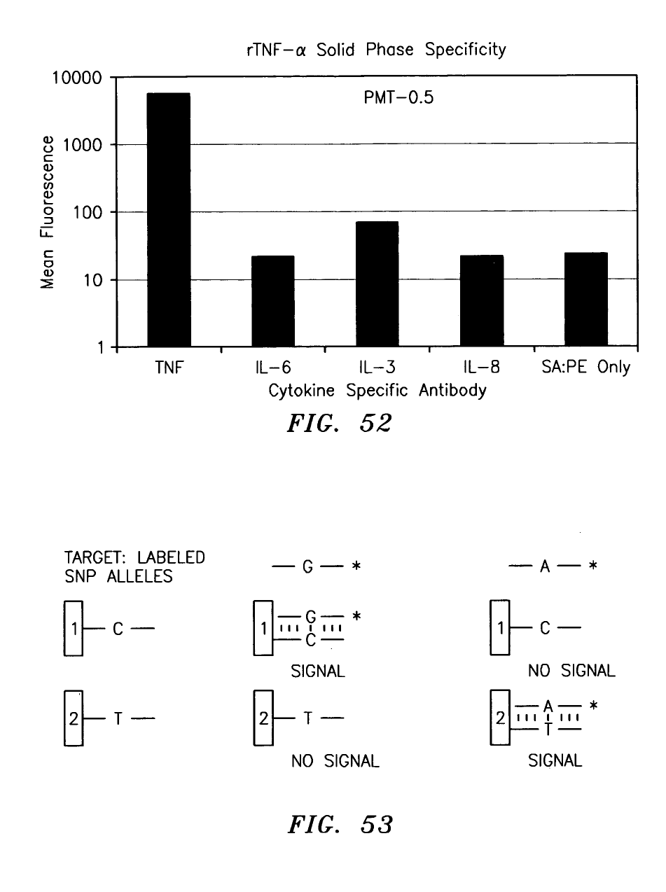

To determine the specificity of the Ag-microbead, a pool of biotinylated secondary antibody was incubated as described above. This pool included the specific anti-TNF-a as well as 3 additional anti-cytokine antibodies (IL-3, IL-6, and IL-8). FIG. 52, which presents the fluorescence signal on a logarithmic scale, shows the high specificity obtained using this format. Non-specific binding of <0.05% was seen.

This example demonstrates the ability to screen for specific antibodies to an analyte in a matrix. In general use, the analyte of interest could be, by way of nonlimiting example, an allergen, infectious disease agent, or antibody.

### Example 15

### Detection of Single Nucleotide Polymorphisms (SNPs) by Allele-Specific Hybridization

Allele specific hybridization utilizes the melting temperature (Tm) differential of an exact match versus a mismatch at a particular SNP position in a sequence. Differential hybridization probes containing either allele are designed such that their Tm's differ by about 10 C. These probes are immobilized to individual beads resulting in one bead specific for the homozygous or wild-type sequence and the other specific for the heterozygous or variant sequence. The beads are mixed with the labeled PCR product incubated at high temperatures to denature the double stranded PCR product and allowed to hybridize at a suitable temperature. Following washes to remove non-specifically hybridized target the beads are examined for fluorescence. An additional step is included if the strand was biotinylated, detect the biotin using, for example, a labeled streptavidin conjugate. A schematic illustration of allele specific hybridization is depicted in FIG. 53. If the sample of interest has a wild-type sequence it will bind to bead 1 which has the complementary probe attached to it. If the sample is heterozygous, both beads 1 and 2 will have signals. A variant allele will be detected on bead 2 only. This chemistry can be performed either in solution and then captured onto a bead via unique sequences, or directly using a specific probe attached to the bead.

### Example 16

### Detection of SNPs by Single Base Chain Extension (SBCE)

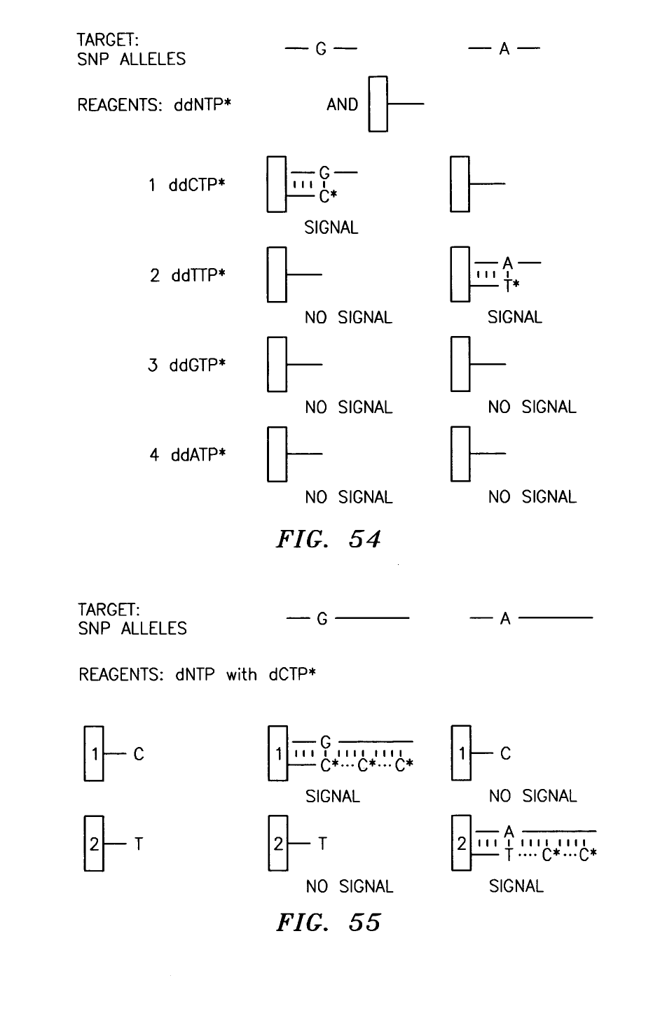

The underlying mechanism of this chemistry is the ability of DNA polymerase to specifically repair errors in replication. In SBCE a probe is annealed to the unknown sequence immediately 3′ of the SNP nucleotide position to be analyzed. Taq polymerase fills in the complementary labeled dideoxynucleotide (ddNTP). (See US patents # 6,004,744 and 5,888,819 owned by Orchid Biosciences.) This method is shown schematically in FIG. 54 with labeled dideoxynucleoside triphosphates. Four labeled ddNTPs must be used for this chemistry resulting in 4 wells per SNP if the SNP is not known. The SBCE reaction can also be done in solution and captured on a digitally encoded bead via a unique targeting DNA probe. A complementary sequence to the bead resides within the primer sequence.

### Example 17

### Detection of SNPs by Allele Specific Primer Extension (ASPE)

This chemistry differs from the SBCE chemistry (Example 16) in that the immobilized probe contains the SNP at its 3′ end. The PCR product is hybridized to the complementary sequences on the beads, the reaction is cycled in the presence of a DNA Polymerase and labeled nucleotides (see FIG. 55). The signal is present if there is a perfect match.

### Example 18

### Detection of SNPs by Oligonucleotide Ligation Assay (OLA)

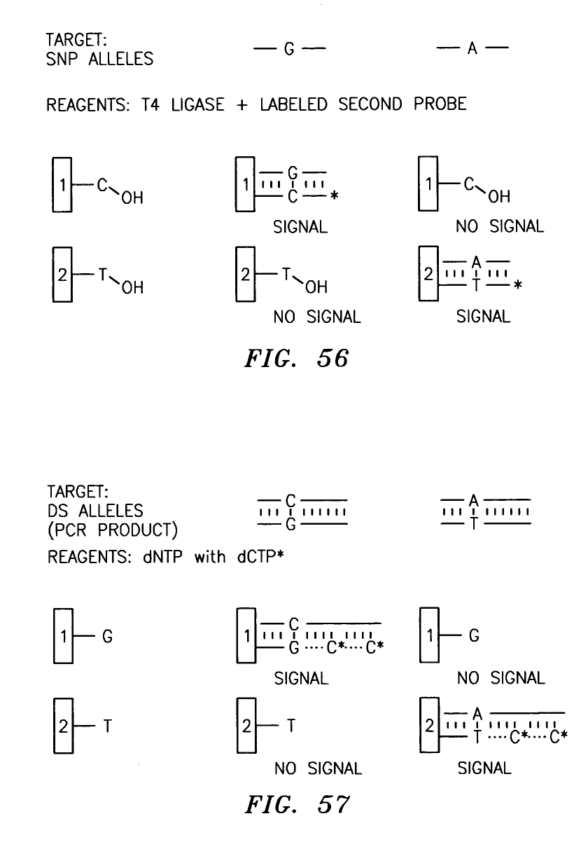

OLA requires two oligonucleotide probes, one immobilized to a bead, and a second labeled and having a 3′ phosphate group (see U.S. Pat. No. 5,869,252 owned by Abbott Laboratories). The PCR product from a suspected SNP is denatured and hybridized with both the immobilized and labeled probes in the presence of T4 Ligase. If there is an exact match the T4 ligase joins the two probes together and a signal is generated (see FIG. 56). The specificity is determined by the mechanism of the T4 Ligase. The ligation chemistry can also be cycled generating multiple copies of the ligated probe.

### Example 19

### Detection of SNPs by Allele Specific PCR (ASPCR)

ASPCR can be used to genotype directly from genomic DNA or from a PCR amplicon. SNP detection is achieved using a primer having the complementary base located at its 3′ end. The other primer is located such that the resulting product is between 80-1000 bp. ASPCR is diagrammed in FIG. 57. Unlike ASPE (Example 17) this chemistry amplifies the number of copies of DNA. This format can also be done on the bead via an attached primer or in solution and then capturing the specific product.

### Example 20

### “React and Combine” in Multiplexed Assays

Generally, different types of beads are mixed together at the assay stage in multiplexed assays. Other assay formats, however, can be envisioned. For example, many assays can be performed separately, each in an individual reaction vessel and perhaps under unique assay conditions, and subsequently mixed together during the read-out step. The constraint on this type of “react and combine” assay is that the particles in each separate reaction must have a set of unique codes particular to each reaction. This allows the particles to be identified correctly during the read step. The simplest example of this sort of “react and combine” multiplexed assay is when each assay is done with a single code per reaction vessel, and all the codes are combined for reading. This is a useful technique to use for assays that may not be chemically compatible during the reaction step.

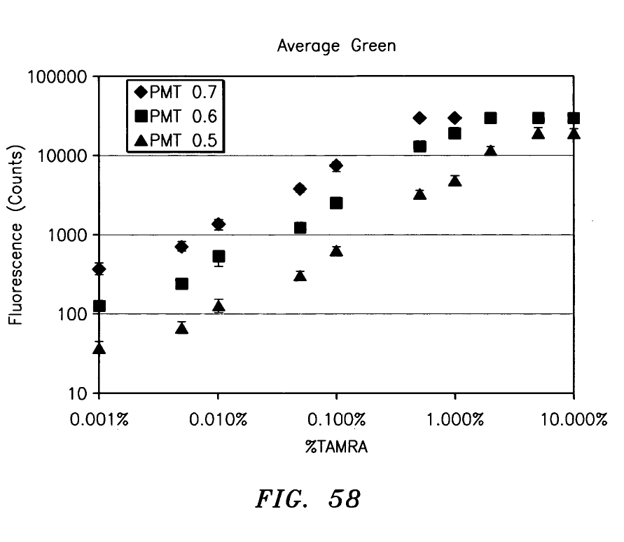

FIG. 58 shows the results of a set of such “react and combine” assays. In this case each data point (at each of 3 photomultiplier (PMT) settings) was the result of attaching a derivatized tetramethylrhodamine (TAMRA; AnaSpec, San Jose, Calif.) dye to the particle surface in a separate reaction. The beads in each TAMRA reaction had a unique code, and were in separate reaction vessels at the time of attachment. After the reaction was completed, excess TAMRA solution was washed off each set of particles. The particles were then combined into a single mixture and read. Since the particles are identified through their codes, no information is lost about each individual reaction. The results, presented in a log-log plot in FIG. 58, show mean values at several TAMRA concentrations at the three PMT settings. A high dynamic range of detection is seen, ranging from 100-fold signal detection at PMT 0.7 to almost 1000-fold at PMT 0.5.

The chemistries, particle dimensions and particle geometries for any of the embodiments described herein are merely for illustrative purposes and, as such, any other dimensions or chemistries may be used if desired, depending on the application, size, performance, manufacturing requirements, or other factors, in view of the teachings herein.

It should be understood that, unless stated otherwise herein, any of the features, characteristics, alternatives or modifications described regarding a particular embodiment herein may also be applied, used, or incorporated with any other embodiment described herein. Also, the drawings herein are not drawn to scale.

Although the invention has been described and illustrated with respect to exemplary embodiments thereof, the foregoing and various other additions and omissions may be made therein and thereto without departing from the spirit and scope of the present invention.

1. A method of identifying the presence and/or amount of an analyte, comprising the steps of:
a) providing an assay article comprising a chemical bound to an optical identification element, wherein the chemical specifically binds to the analyte and wherein the optical identification element comprises:
i) an optical substrate;
ii) the chemical being bound to said substrate; and
iii) at least a portion of said substrate having at least one diffraction grating disposed therein, said grating having at least one refractive index pitch superimposed at a common location;
wherein the grating provides an output optical signal when illuminated by an incident light signal; and wherein said optical output signal is indicative of a code in said substrate;
b) contacting the assay article with a sample containing the analyte, thereby binding the analyte to the assay article;
c) determining the code provided by the assay article; and
d) determining the presence and/or amount of the analyte bound to the assay article thereby identifying the presence and/or amount of the analyte.

2. The method described in claim 1 wherein the assay article is a particle or bead.

3. The method described in claim 1 wherein the chemical is bound to the article by a covalent bond.

4. The method described in claim 1 wherein the chemical comprises a nucleic acid, a polynucleotide, an oligonucleotide, a nucleotide, a nucleoside, a protein nucleic acid, an oligopeptide nucleic acid, a protein or fragment thereof, an antibody or fragment thereof, an enzyme or fragment thereof, a receptor or fragment thereof, a polypeptide, an oligopeptide, an amino acid, a derivative of any of them, or a modification of any of them.

5. The method described in claim 1 wherein the chemical comprises a moiety chosen from the group consisting of a synthetic organic molecule, a synthetic intermediate, a synthetic precursor, an antibiotic, a metabolite, a candidate pharmaceutical agent, or a pharmaceutical agent.

6. The method described in claim 1 wherein the chemical comprises a moiety chosen from the group consisting of a virus, a prokaryotic cell, a eukaryotic cell, a vertebrate cell, a mammalian cell, a human cell, a subcellular organelle, and a component of any them.

7. The method described in claim 1 wherein the analyte comprises a polynucleotide that comprises an allele of a single nucleotide polymorphism and the chemical comprises a sequence complementary to a sequence comprising the single nucleotide polymorphism.

8. The method described in claim 1 further comprising a linker between the substrate and a moiety comprising the chemical.

9. The method described in claim 8 wherein the moiety further comprises a spacer that binds the moiety to the linker.

10. The method described in claim 1 wherein a moiety comprising the chemical further comprises a spacer that binds the moiety to the substrate.

11. The method described in claim 1 wherein the chemical is bound to the article by noncovalent interactions.

12. The method described in claim 1 wherein the analyte is labeled.

13. The method described in claim 12 wherein the presence and/or amount of the label is determined.

14. The method described in claim 12 wherein the label emits radiation and the presence and/or intensity of the radiation is determined.

15. The method described in claim 1 wherein determining the presence and/or amount of the analyte further comprises binding a specific detecting substance to the bound analyte and determining the specific detecting substance.

16. The method described in claim 15 wherein the specific detecting substance is labeled.

17. The method described in claim 1 wherein the substrate comprises silica, a silicate, a glass, a semiconducting material, or a ceramic material.

18. The method described in claim 1 wherein the substrate comprises a polymer, a resin, a rubber material, or a derivative thereof.

19. The method of claim 1 wherein at least one refractive index pitch superimposed at said grating location provides a refractive index variation.

20. The method of claim 1 wherein a plurality of refractive index pitches superimposed at said grating location provides a refractive index variation.

21. The method of claim 1 wherein said code comprises a plurality of digital bits.

22. The method of claim 1 wherein said code comprises a plurality of bits, each bit having a plurality of states.

23. The method of claim 1 wherein said code comprises a plurality of bits, each bit having a corresponding spatial location and each bit in said code having a value related to the intensity of said output optical signal at the spatial location of each bit.

24. The method of claim 23 wherein the value of each bit corresponds to the magnitude of refractive index variation of a corresponding refractive index pitch in said grating.

25. The method of claim 1 wherein said code comprises a plurality of digital bits, each bit having a corresponding spatial location and each bit in said code having a binary value related to the intensity of said output optical signal at the spatial location of each bit.

26. The method of claim 26 wherein the value of each bit corresponds to the presence or absence of a corresponding refractive index pitch in said grating.

27. The method of claim 1 wherein said incident light signal comprises a single wavelength.

28. The method of claim 1 wherein said substrate has a grating region where said grating is located and a non-grating region where said grating is not located; and wherein said substrate has a plurality of grating regions.

29. The method of claim 1 wherein said substrate comprises a plurality of said gratings.

30. The method of claim 1 wherein said substrate comprises a plurality of said gratings each at different locations within said substrate.

31. A method of conducting a multiplexed assay for the presence and/or amount of one or more analytes, comprising the steps of:
a) providing a plurality of assay articles wherein an assay article comprises a chemical bound to an optical identification element, wherein each chemical specifically binds to an analyte, and wherein each optical identification element comprises:
i) an optical substrate;
ii) at least a portion of said substrate having at least one diffraction grating disposed therein, said grating having at least one refractive index pitch superimposed at a common location;
iii) the grating providing an output optical signal when illuminated by an incident light signal;
wherein said optical output signal is indicative of a first code in said substrate of a first assay article, and said first code differs from a second code provided by a second assay article; and
wherein a first chemical bound to the substrate of the first assay article is identified by the first code provided thereby, and a second chemical bound to the substrate of the second assay article is identified by the second code provided thereby;
b) contacting the plurality of assay articles with a sample containing one or more analytes, thereby binding an analyte to an assay article to provide a positive assay article;
c) determining the code provided by the positive assay article; and
d) determining the presence and/or amount of an analyte bound to the positive assay article.

32. A method of identifying the occurrence of a process wherein the process requires an analyte and provides a detectable label bound to an assay article, comprising the steps of:
a) providing an assay article comprising a chemical bound to an optical identification element, wherein the chemical binds to the analyte, and wherein the optical identification element comprises:
i) an optical substrate;
ii) the chemical being bound to said substrate;
iii) at least a portion of said substrate having at least one diffraction grating disposed therein, said grating having at least one refractive index pitch superimposed at a common location; and
iv) the grating providing an output optical signal when illuminated by an incident light signal;
wherein said optical output signal is indicative of a code in said substrate;
b) contacting the assay article with a sample containing the analyte and a component that permits the process to occur, thereby binding the label to the assay article;
c) determining the code characterizing the assay article; and
d) determining the presence of the label bound to the assay article;
thereby identifying the occurrence of the process.

33. The method described in claim 32 wherein the process labels the chemical.

34. The method described in claim 32 wherein the process labels the analyte.

35. A method of conducting a multiplexed assay for identifying the occurrence of a process wherein the process requires an analyte and provides a detectable label bound to an assay article, comprising the steps of:
a) providing a plurality of assay articles wherein an assay article comprises a chemical bound to an optical identification element, wherein the chemical binds to the analyte, and wherein the optical identification elements comprise:
i) an optical substrate;
ii) at least a portion of said substrate having at least one diffraction grating disposed therein, said grating having at least one refractive index pitch superimposed at a common location; and
iii) the grating providing an output optical signal when illuminated by an incident light signal;
wherein said optical output signal is indicative of a first code in said substrate of a first assay article, and said first code differs from a second code provided by a second assay article; and
wherein a first chemical is bound to the substrate of the first assay article and is identified by the first code provided thereby, and a second chemical is bound to the substrate of the second assay article and is identified by the second code provided thereby;
b) contacting the plurality of assay articles with a sample containing the analyte and a component that permits the process to occur, thereby binding a label to at least one assay article;
c) determining the code provided by the at least one assay article; and
d) determining the presence of a label bound to the at least one assay article;
thereby identifying the occurrence of the process.

36. The method described in claim 35 wherein the process labels the chemical.

37. The method described in claim 35 wherein the process labels the analyte.

38. An assay article comprising a chemical bound to an optical identification element, said chemical specifically binding to an analyte, wherein the optical identification element comprises:
a) an optical substrate;
b) the chemical being bound to said substrate;
c) at least a portion of said substrate having at least one diffraction grating disposed therein, said grating having at least one refractive index pitch superimposed at a common location; and
d) the grating providing an output optical signal when illuminated by an incident light signal;
wherein said optical output signal is indicative of a code in said substrate.

39. The assay article described in claim 38 wherein the assay article is a particle or bead.

40. The assay article described in claim 38 wherein the chemical is bound to the article by a covalent bond.

41. The assay article described in claim 38 wherein the chemical comprises a nucleic acid, a polynucleotide, an oligonucleotide, a nucleotide, a nucleoside, a protein nucleic acid, an oligopeptide nucleic acid, a protein or fragment thereof, an antibody or fragment thereof, an enzyme or fragment thereof, a receptor or fragment thereof, a polypeptide, an oligopeptide, an amino acid, a derivative of any of them, or a modification of any of them.

42. The assay article described in claim 38 wherein the chemical comprises a moiety chosen from the group consisting of a synthetic organic molecule, a synthetic intermediate, a synthetic precursor, an antibiotic, a metabolite, a candidate pharmaceutical agent, or a pharmaceutical agent.

43. The assay article described in claim 38 wherein the chemical comprises a moiety chosen from the group consisting of a virus, a prokaryotic cell, a eukaryotic cell, a vertebrate cell, a mammalian cell, a human cell, a subcellular organelle, and a component of any them.

44. The assay article described in claim 38 further comprising a linker between the substrate and a moiety comprising the chemical.

45. The assay article described in claim 44 wherein the moiety further comprises a spacer that binds the moiety to the linker.

46. The assay article described in claim 38 wherein a moiety comprising the chemical further comprises a spacer that binds the moiety to the substrate.

47. The assay article described in claim 38 wherein the chemical is bound to the article by noncovalent interactions.

48. The assay article described in claim 38 wherein the analyte is labeled.

49. The assay article described in claim 48 wherein the presence and/or amount of the label is determined.

50. The assay article described in claim 48 wherein the label emits radiation and the presence and/or intensity of the radiation is determined.

51. The assay article described in claim 38 wherein the substrate comprises silica, a silicate, a glass, a semiconducting material, or a ceramic material.

52. The assay article described in claim 38 wherein the substrate comprises a polymer, a resin, a rubber material, or a derivative thereof.

53. The assay article of claim 38 wherein at least one refractive index pitch superimposed at said grating location provides a refractive index variation.

54. The assay article of claim 38 wherein a plurality of refractive index pitches superimposed at said grating location provides a refractive index variation.

55. The assay article of claim 38 wherein said code comprises a plurality of digital bits.

56. The assay article of claim 38 wherein said code comprises a plurality of bits, each bit having a plurality of states.

57. The assay article of claim 38 wherein said code comprises a plurality of bits, each bit having a corresponding spatial location and each bit in said code having a value related to the intensity of said output optical signal at the spatial location of each bit.

58. The assay article of claim 57 wherein the value of each bit corresponds to the magnitude of refractive index variation of a corresponding refractive index pitch in said grating.

59. The assay article of claim 38 wherein said code comprises a plurality of digital bits, each bit having a corresponding spatial location and each bit in said code having a binary value related to the intensity of said output optical signal at the spatial location of each bit.

60. The assay article of claim 59 wherein the value of each bit corresponds to the presence or absence of a corresponding refractive index pitch in said grating.

61. The assay article of claim 38 wherein said incident light signal comprises a single wavelength.

62. The assay article of claim 38 wherein said substrate has a grating region where said grating is located and a non-grating region where said grating is not located; and wherein said substrate has a plurality of grating regions.

63. The assay article of claim 38 wherein said substrate comprises a plurality of said gratings.

64. The assay article of claim 38 wherein said substrate comprises a plurality of said gratings each at different locations within said substrate.

65. A set comprising a plurality of assay articles wherein each assay article comprises a chemical bound to an optical identification element, said chemical specifically binding to an analyte, wherein each optical identification element comprises:
a) an optical substrate;
b) at least a portion of said substrate having at least one diffraction grating disposed therein, said grating having at least one refractive index pitch superimposed at a common location;
c) the grating providing an output optical signal when illuminated by an incident light signal;
wherein said optical output signal is indicative of a first code in said substrate of a first assay article, and said first code differs from a second code provided by a second assay article; and
wherein a first chemical bound to the substrate of the first assay article is identified by the first code provided thereby, and a second chemical bound to the substrate of the second assay article is identified by the second code provided thereby.

66. An assay article comprising a specific binding pair bound to an optical identification element, said specific binding pair comprising a first specific binding substance bound to a cognate specific binding substance, wherein the optical identification element comprises:
a) an optical substrate;
b) the first specific binding substance being bound to said substrate;
c) at least a portion of said substrate having at least one diffraction grating disposed therein, said grating having at least one refractive index pitch superimposed at a common location; and
d) the grating providing an output optical signal when illuminated by an incident light signal;
wherein said optical output signal is indicative of a code in said substrate.

67. The assay article described in claim 66 wherein the first specific binding substance is a chemical and the second specific binding substance is an analyte specifically bound by the chemical.

68. The assay article described in claim 66 wherein the first specific binding substance is a receptor and the second specific binding substance is a ligand specifically bound by the receptor.

69. The assay article described in claim 66 wherein the first specific binding substance is a probe and the second specific binding substance is a target specifically bound by the probe.

70. The assay article described in claim 69 wherein the target comprises a polynucleotide that comprises an allele of a single nucleotide polymorphism and the probe comprises a sequence complementary to a sequence comprising the single nucleotide polymorphism.

71. The assay article described in claim 66 wherein the cognate specific binding substance is labeled.

72. The assay article described in claim 66 wherein the cognate specific binding substance further binds to a specific detecting substance.

73. The assay article described in claim 72 wherein the specific detecting substance is labeled.

74. A set containing a specific binding pair comprising:
1) at least one first assay article comprising a specific binding pair bound to an optical identification element, said specific binding pair comprising a first specific binding substance bound to a cognate specific binding substance; and
2) at least one second assay article wherein each second assay article comprises a second specific binding substance bound to an optical identification element; and
wherein each optical identification element comprises:
a) an optical substrate;
b) at least a portion of said substrate having at least one diffraction grating disposed therein, said grating having at least one refractive index pitch superimposed at a common location;
c) the grating providing an output optical signal when illuminated by an incident light signal;
wherein said optical output signal is indicative of a first code in said substrate of the first assay article, and said first code differs from a second code provided by the second assay article; and
wherein the first specific binding substance is bound to the substrate of the first assay article and is identified by the first code provided thereby, and
the second specific binding substance is bound to the substrate of the second assay article and is identified by the second code provided thereby.

75. The set described in claim 74 wherein the first specific binding substance is a chemical and the cognate specific binding substance is an analyte.

76. The set described in claim 74 wherein the first specific binding substance is a receptor and the cognate specific binding substance is a ligand.

77. The set described in claim 74 wherein the first specific binding substance is a probe and the cognate specific binding substance is a target.

78. The set described in claim 74 wherein the first specific binding substance is labeled.

79. The set described in claim 74 wherein the cognate specific binding substance is labeled.

80. A method of analyzing a target substance in a sample comprising:
a) contacting the sample with a plurality of coded assay articles bearing probe substances,
wherein a probe substance specifically binds a target substance, thereby binding the target substance to a coded assay article,
wherein each coded assay article comprises the probe substance bound to an optical substrate, and
wherein at least a portion of said substrate has at least one diffraction grating disposed therein, said grating having at least one refractive index pitch superimposed at a common location;
wherein the grating provides an output optical signal when illuminated by an incident light signal; and wherein said optical output signal is indicative of the code in said substrate;
b determining the code provided by the assay article; and
c) analyzing the target substance bound to the coded assay article.


SEQUENCE LISTING (15 sequences)

SEQ ID NO: 1 - DNA, 26 bp, Artificial Sequence
  Caenorhabditis elegans
gcgttttacaataacttctcgtgcca

SEQ ID NO: 2 - DNA, 26 bp, Artificial Sequence
  Caenorhabditis elegans
gcgttatagattaacctctcctgcca

SEQ ID NO: 3 - DNA, 26 bp, Artificial Sequence
  Caenorhabditis elegans
tcaaaataccattgcagctaccattt

SEQ ID NO: 4 - DNA, 26 bp, Artificial Sequence
  Caenorhabditis elegans
gtgcgttttacaataacttccgtgcg

SEQ ID NO: 5 - DNA, 66 bp, Artificial Sequence
  Virus PhiX174
gccctggtcgtccgcagccgttgcgaggtactaaaggcaagcgtaaaggcgctcgtctttggtatg

SEQ ID NO: 6 - DNA, 66 bp, Artificial Sequence
  Virus PhiX174
cataccaaagacgagcgcctttacgcttgcctttagtacctcgcaacggctgcggacgaccagggc

SEQ ID NO: 7 - DNA, 67 bp, Artificial Sequence
  Virus PhiX174
attagcataagcagcttgcagacccataatgtcaatagatgtggtagaagtcgtcatttggcgagaa

SEQ ID NO: 8 - DNA, 67 bp, Artificial Sequence
  Virus PhiX174
ttctcgccaaatgacgacttctaccacatctattgacattatgggtctgcaagctgcttatgctaat

SEQ ID NO: 9 - DNA, 66 bp, Artificial Sequence
  Virus PhiX174
catttcctgagcttaatgcttgggagcgtgctggtgctgatgcttcctctgctggtatggttgacg

SEQ ID NO: 10 - DNA, 67 bp, Artificial Sequence
  Virus PhiX174
caagtatcggcaacagctttatcaataccatgaaaaatatcaaccacaccagaagcagcatcagtga

SEQ ID NO: 11 - DNA, 67 bp, Artificial Sequence
  Virus PhiX174
gcgcggtaggttttctgcttaggagtttaatcatgtttcagacttttatttctcgccataattcaaa

SEQ ID NO: 12 - DNA, 67 bp, Artificial Sequence
  Virus PhiX174
gagaaataaaagtctgaaacatgattaaactcctaagcagaaaacctaccgcgcttcgcttggtcaa

SEQ ID NO: 13 - DNA, 70 bp, Artificial Sequence
  Homo sapiens
catccgacattgaagttgacttactgaagaatggagagagaattgaaaaagtggagcattcagacttgtc

SEQ ID NO: 14 - DNA, 70 bp, Artificial Sequence
  Homo sapiens
atgtcgcggtttttcaccaccggttcggacagcgagtccgagtcgtccttgtccggggaggagctcgtca

SEQ ID NO: 15 - DNA, 70 bp, Artificial Sequence
  Homo sapiens
agagaacttcaaaaaaccaactagaagcaacatgcagagaagtaaaatgagaggggcctcctcaggaaag